SEQUENCE LISTING (41 sequences)

SEQ ID NO: 1 - DNA, 2088 bp, Lemna minor
  5′UTR (1)..(40): 5′UTR of threonine deaminase (TD) isoform #1
ctctcggatcctgcatcgtcttcctcgtccctcgatcctcatggcggcgctgcagatccttccccggccacaggcgccttgttccggccgatctccagcgccttctccggcttcttccgccgccacttgctgcacaatgtccagatccccatccatatccttaaagcggtgttcttgctatcgatatccctctcgttactcccatggcatccccagtgatggcggaatcagaggcaaattgacctcatctgctgttcccgccgcatcatttgcttctccttccaccaccgccgacgcccctagcgatgccgcaacagctccattgtcgaccccatccgtctcttctgaggcctccgccgaagttgaattgatgaaggtcaccaccgactcgcttcagtatgagagtgggtatctcgggggcatttccggaaaaactcgtccctcttgggggacgagctggacgagcagtccatcgagcttcgacaggccgagcgccatggattacttagctcacactctcacctccagagtctacgatgtggccatcgaatcccccctccagctcgctcccaggctttccgagcggctcggtgtgcagttctggctgaagcgcgaagatctgcaaccagtgttctcattcaaattgcgaggagcgtataatatgatggcgaatcttcctagagaaaagctggaaaaaggagtaatatgttcttcagcagggaatcacgctcaaggagttgctctggctgcacagaaactaggctgcaatgcagtgatcgtcatgcccgttactacgccagaaatcaagtggaaatctgttgaaaaattgggcgcaactgttgttcttgtgggagattcttacgatgaagcgcaatcgcatgccaagaaaagagcaaaatcggagggccgcactttcattccgcctttcgataaccctaacgtcataatgggccaaggaactgttggaatggagatcatcaggcaattgagaggcccgattcatgccatctttgtacccgttggtggtggtctgattgctggaattgcagcttatgtgaaacaagtccgccctgaggtgaagatcatcggtgtggaaccatacgatgcaaatgccatggcgttatcgttgcatcatgggcagagggtcatgctcgagcaagtgggcggtttcgcagatggtgttgctgttaaagtcgtcggcgaagaaacttatcgcctatgccgagaactagttgatggtattgttcttgtcagtcgcgatgcaatttgtgcatctataaaggacatgttcgaggaaaagaggagtatcctcgagccagccggtgcactctcattggccggtgcagaagcttactgcaaatactacggtctgaagggggaatctgtggtagccatcacatcgggcgcaaacatgaactttgatcggttgcgattggttaccgagcttgctgatgtgggccgtaaacaagaagctgttctcgccacttccatgccggaagaacccggaagcttcaaaagattctgtcagctggtgggcccggtgaatatcaccgagttcaagtaccggtacgatgctagcaaggagaaggctcttgttctttacagtgttggagtgcatactgctgcggagcttaagtctgtggtaggccgcatggggtttgaaaattttgagactgttgatcttacgaataatgacttggccaaagatcatcttcgtcatctggttgggggtcggacaaatgtggagaatgagctgctgtgtagattcatcttcccggagaggcctggcaccctgatgaagttcctcgactccttcagcccgcgctggaacatcagtctcttccactatcgatcccagggggaggccggggcaaatgttctggttggaatccaggtacctggaggcgagatggacgagttccgcgccatcgccaccaacctagactatgattatgccctagagatgtccaacaaggcttaccagctcctcatgcactgaaccatgggcctaaccctaatttattgcagatgatgatgatgataatgatgatgatgatagttgtgttgtatgcggtgttatggcttctg

SEQ ID NO: 2 - DNA, 1959 bp, Lemna minor
  CDS (1)..(1959): Encodes threonine deaminase (TD) isoform #1
atggcggcgctgcagatccttccccggccacaggcgccttgttccggccgatctccagcgccttctccggcttcttccgccgccacttgctgcacaatgtccagatccccatccatatccttaaagcggtgttcttgctatcgatatccctctcgttactcccatggcatccccagtgatggcggaatcagaggcaaattgacctcatctgctgttcccgccgcatcatttgcttctccttccaccaccgccgacgcccctagcgatgccgcaacagctccattgtcgaccccatccgtctcttctgaggcctccgccgaagttgaattgatgaaggtcaccaccgactcgcttcagtatgagagtgggtatctcgggggcatttccggaaaaactcgtccctcttgggggacgagctggacgagcagtccatcgagcttcgacaggccgagcgccatggattacttagctcacactctcacctccagagtctacgatgtggccatcgaatcccccctccagctcgctcccaggctttccgagcggctcggtgtgcagttctggctgaagcgcgaagatctgcaaccagtgttctcattcaaattgcgaggagcgtataatatgatggcgaatcttcctagagaaaagctggaaaaaggagtaatatgttcttcagcagggaatcacgctcaaggagttgctctggctgcacagaaactaggctgcaatgcagtgatcgtcatgcccgttactacgccagaaatcaagtggaaatctgttgaaaaattgggcgcaactgttgttcttgtgggagattcttacgatgaagcgcaatcgcatgccaagaaaagagcaaaatcggagggccgcactttcattccgcctttcgataaccctaacgtcataatgggccaaggaactgttggaatggagatcatcaggcaattgagaggcccgattcatgccatctttgtacccgttggtggtggtctgattgctggaattgcagcttatgtgaaacaagtccgccctgaggtgaagatcatcggtgtggaaccatacgatgcaaatgccatggcgttatcgttgcatcatgggcagagggtcatgctcgagcaagtgggcggtttcgcagatggtgttgctgttaaagtcgtcggcgaagaaacttatcgcctatgccgagaactagttgatggtattgttcttgtcagtcgcgatgcaatttgtgcatctataaaggacatgttcgaggaaaagaggagtatcctcgagccagccggtgcactctcattggccggtgcagaagcttactgcaaatactacggtctgaagggggaatctgtggtagccatcacatcgggcgcaaacatgaactttgatcggttgcgattggttaccgagcttgctgatgtgggccgtaaacaagaagctgttctcgccacttccatgccggaagaacccggaagcttcaaaagattctgtcagctggtgggcccggtgaatatcaccgagttcaagtaccggtacgatgctagcaaggagaaggctcttgttctttacagtgttggagtgcatactgctgcggagcttaagtctgtggtaggccgcatggggtttgaaaattttgagactgttgatcttacgaataatgacttggccaaagatcatcttcgtcatctggttgggggtcggacaaatgtggagaatgagctgctgtgtagattcatcttcccggagaggcctggcaccctgatgaagttcctcgactccttcagcccgcgctggaacatcagtctcttccactatcgatcccagggggaggccggggcaaatgttctggttggaatccaggtacctggaggcgagatggacgagttccgcgccatcgccaccaacctagactatgattatgccctagagatgtccaacaaggcttaccagctcctcatgcactga

SEQ ID NO: 3 - protein, 652 aa, Lemna minor
MAALQILPRPQAPCSGRSPAPSPASSAATCCTMSRSPSISLKRCSCYRYPSRYSHGIPSDGGIRGKLTSSAVPAASFASPSTTADAPSDAATAPLSTPSVSSEASAEVELMKVTTDSLQYESGYLGGISGKTRPSWGTSWTSSPSSFDRPSAMDYLAHTLTSRVYDVAIESPLQLAPRLSERLGVQFWLKREDLQPVFSFKLRGAYNMMANLPREKLEKGVICSSAGNHAQGVALAAQKLGCNAVIVMPVTTPEIKWKSVEKLGATVVLVGDSYDEAQSHAKKRAKSEGRTFIPPFDNPNVIMGQGTVGMEIIRQLRGPIHAIFVPVGGGLIAGIAAYVKQVRPEVKIIGVEPYDANAMALSLHHGQRVMLEQVGGFADGVAVKVVGEETYRLCRELVDGIVLVSRDAICASIKDMFEEKRSILEPAGALSLAGAEAYCKYYGLKGESVVAITSGANMNFDRLRLVTELADVGRKQEAVLATSMPEEPGSFKRFCQLVGPVNITEFKYRYDASKEKALVLYSVGVHTAAELKSVVGRMGFENFETVDLTNNDLAKDHLRHLVGGRTNVENELLCRFIFPERPGTLMKFLDSFSPRWNISLFHYRSQGEAGANVLVGIQVPGGEMDEFRAIATNLDYDYALEMSNKAYQLLMH

SEQ ID NO: 4 - DNA, 2091 bp, Lemna minor
  5′UTR (1)..(40): 5′UTR for threonine deaminase (TD) isoform #2
ctctcggatcctgcatcgtcttcctcgtccctcgatcctcatggcggcgctgcagatccttccccggccacaggcgccttgttccggccgatctccagcgccttctccggcttcttccgccgccacttgctgcacaatgtccagatccccatccatatccttaaagcggtgttcttgctatcgatatccctctcgttactcccatggcatccccagtgatggcggaatcagaggcaaattgacctcatctgctgtttccgccgcatcatttgcttctccttccaccaccgccgacgcccctagcgatgccgcaacagctccattgtcgaccccatccgtctcttctgaggcctccgccgaagttgaattgatgaaggtcaccaccgactcgcttcagtatgagagtgggtatctcgggggcatttccggaaaaactcgtccctcttgggggacgagctggacgagcagtccatcgagcttcgacaggccgagcgccatggattacttagctcacactctcacctccagagtctacgatgtggccatcgaatcccccctccagctcgctcccaggctttccgagcggctcggtgtgcagttctggctgaagcgcgaagatctgcaaccagtgttctcattcaaattgcgaggagcgtataatatgatggcgaatcttcctagagaaaagctggaaaaaggagtaatatgttcttcagcagggaatcacgctcaaggagttgctctggctgcacagaaactaggctgcaatgcagtgatcgtcatgcccgttactacgccagaaatcaagtggaaatctgttgaaaaattgggcgcaactgttgttcttgtgggagattcttacgatgaagcgcaatcgcatgccaagaaaagagcaaaatcggagggccgcactttcattccgcctttcgataaccctaacgtcataatgggccaaggaactgttggaatggagatcatcaggcaattgagaggcccgattcatgccatctttgtacccgttggtggtggtggtctgattgctggaattgcagcttatgtgaaacaagtccgccctgaggtgaagatcatcggtgtggaaccatacgatgcaaatgccatggcgttatcgttgcatcatgggcagagggtcatgctcgagcaagtgggcggtttcgcagatggtgttgctgttaaagtcgtcggcgaagaaacttatcgcctatgccgagaactagttgatggtattgttcttgtcagtcgcgatgcaatttgtgcatctataaaggacatgttcgaggaaaagaggagtatcctcgagccagccggtgcactctcattggccggtgcagaagcttactgcaaatactacggtctgaagggggaatctgtggtagccatcacatcgggcgcaaacatgaactttgatcggttgcgattggttacctagcttgctgatgtgggccgtaaacaagaagctgttctcgccacttccatgccggaagaacccggaagcttcaaaagattctgtcagctggtgggcccggtgaatatcaccgagttcaagtaccggtacgatgctagcaaggagaaggctcttgttctttacagtgttggagtgcatactgctgcggagcttaagtctatggtaggccgcatggagtttgaaaattttgagactgttgatcttacgaataatgacttggccaaagatcatcttcgtcatctggttgggggtcggacaaatgtggagaatgagctgctgtgtagattcatcttcccggagaggcctggcaccctgatgaagttcctcgactccttcagcccgcgctggaacatcagtctcttccactatcgatcccagggggaggccggggcaaatgttctggttggaatccaggtacctggaggcgagatggacgagttccgcgccatcgccaccaacctagactatgattatgccctagagatgtccaacaaggcttaccagctcctcatgcactgaaccatgggcctaaccctaatttattgcagatgatgatgatgataatgatgatgatgatagttgtgttgtatgcggtgttatggcttctg

SEQ ID NO: 5 - DNA, 1407 bp, Lemna minor
  CDS (1)..(1407): Encodes threonine deaminase (TD) isoform #2
atggcggcgctgcagatccttccccggccacaggcgccttgttccggccgatctccagcgccttctccggcttcttccgccgccacttgctgcacaatgtccagatccccatccatatccttaaagcggtgttcttgctatcgatatccctctcgttactcccatggcatccccagtgatggcggaatcagaggcaaattgacctcatctgctgtttccgccgcatcatttgcttctccttccaccaccgccgacgcccctagcgatgccgcaacagctccattgtcgaccccatccgtctcttctgaggcctccgccgaagttgaattgatgaaggtcaccaccgactcgcttcagtatgagagtgggtatctcgggggcatttccggaaaaactcgtccctcttgggggacgagctggacgagcagtccatcgagcttcgacaggccgagcgccatggattacttagctcacactctcacctccagagtctacgatgtggccatcgaatcccccctccagctcgctcccaggctttccgagcggctcggtgtgcagttctggctgaagcgcgaagatctgcaaccagtgttctcattcaaattgcgaggagcgtataatatgatggcgaatcttcctagagaaaagctggaaaaaggagtaatatgttcttcagcagggaatcacgctcaaggagttgctctggctgcacagaaactaggctgcaatgcagtgatcgtcatgcccgttactacgccagaaatcaagtggaaatctgttgaaaaattgggcgcaactgttgttcttgtgggagattcttacgatgaagcgcaatcgcatgccaagaaaagagcaaaatcggagggccgcactttcattccgcctttcgataaccctaacgtcataatgggccaaggaactgttggaatggagatcatcaggcaattgagaggcccgattcatgccatctttgtacccgttggtggtggtggtctgattgctggaattgcagcttatgtgaaacaagtccgccctgaggtgaagatcatcggtgtggaaccatacgatgcaaatgccatggcgttatcgttgcatcatgggcagagggtcatgctcgagcaagtgggcggtttcgcagatggtgttgctgttaaagtcgtcggcgaagaaacttatcgcctatgccgagaactagttgatggtattgttcttgtcagtcgcgatgcaatttgtgcatctataaaggacatgttcgaggaaaagaggagtatcctcgagccagccggtgcactctcattggccggtgcagaagcttactgcaaatactacggtctgaagggggaatctgtggtagccatcacatcgggcgcaaacatgaactttgatcggttgcgattggttacctag

SEQ ID NO: 6 - protein, 468 aa, Lemna minor
MAALQILPRPQAPCSGRSPAPSPASSAATCCTMSRSPSISLKRCSCYRYPSRYSHGIPSDGGIRGKLTSSAVSAASFASPSTTADAPSDAATAPLSTPSVSSEASAEVELMKVTTDSLQYESGYLGGISGKTRPSWGTSWTSSPSSFDRPSAMDYLAHTLTSRVYDVAIESPLQLAPRLSERLGVQFWLKREDLQPVFSFKLRGAYNMMANLPREKLEKGVICSSAGNHAQGVALAAQKLGCNAVIVMPVTTPEIKWKSVEKLGATVVLVGDSYDEAQSHAKKRAKSEGRTFIPPFDNPNVIMGQGTVGMEIIRQLRGPIHAIFVPVGGGGLIAGIAAYVKQVRPEVKIIGVEPYDANAMALSLHHGQRVMLEQVGGFADGVAVKVVGEETYRLCRELVDGIVLVSRDAICASIKDMFEEKRSILEPAGALSLAGAEAYCKYYGLKGESVVAITSGANMNFDRLRLVT

SEQ ID NO: 7 - DNA, 1236 bp, Lemna minor
  5′UTR (1)..(33): 5′UTR for glutamine synthetase 1 (GS1) isoform #1
caatttcctctgctcccgcttctgatccctgcaatggctcttctcgccgatctccagaacctgaatctcaccgagaccacggagaagatcatcgccgagtacatatggatcggcggctctggcttggacatgaggagcaaggcgaggacgatctccaaaccggtgtctgatcccaaggaactccccaagtggaactacgacggctccagcactggtcaagctcctggagaggacagcgaagtgatcctctatccccaggccatcttcagggatccattcaggaagggaaacaacattcttgtgatgtgcgactgctacacgccagcgggagagccgatccccacgaacaagaggtacagagcctctcagatcttcagcggtcccgccgtcgtcgcagaagagacctggtatggactagagcaggagtacactctactccagaaggacgtgaagtggcctctgggctggcctctgggcggcttccctgctccacagggtccgtactactgcggtataggcgtggacaaggcgttcgggagagagatcgtcgacgcccactacaaggcctgcctgtacgcaggaatcaacatcagcgggatcaatggcgaagtcatgcctggacagtgggagttccaagttggaccatccgtcggaatctcagcctccgatcagctctggatcgctcgctacctcttggagaggatcacagaggtcgccggagttgttctctccttgcaccccaagccaatcaagggtgactggaacggcgctggatgccacaccaactacagtaccaaatcgatgagggaggatggcggatacgagctgatcaagaaggcgatcgacaagctcggactcaggcacaaggaacacatcgaggcctacggcgaggataacgaggagcgtctcactggccgccacgagaccgccgacatccacaccttcaaatggggcgtggccaaccggggagcttcgatccgcgtcggacgggacacggagaaggaaggaaaaggttacttcgaggacaggaggccggcttccaacatggacccgtacgtggtgacctccatgatcgcggagacgacccttctctggaagccctgatcgcggagcttcttccatggtcgtcctgctcgtccttctctttcaatttctgtcaaaaatggctggcttttccatccttctggatgtgagtctgtgtcggcggggtgagtgatcggttgacttctccccctc

SEQ ID NO: 8 - DNA, 1071 bp, Lemna minor
  CDS (1)..(1071): Encodes glutamine synthetase 1 (GS1) isoform #1
atggctcttctcgccgatctccagaacctgaatctcaccgagaccacggagaagatcatcgccgagtacatatggatcggcggctctggcttggacatgaggagcaaggcgaggacgatctccaaaccggtgtctgatcccaaggaactccccaagtggaactacgacggctccagcactggtcaagctcctggagaggacagcgaagtgatcctctatccccaggccatcttcagggatccattcaggaagggaaacaacattcttgtgatgtgcgactgctacacgccagcgggagagccgatccccacgaacaagaggtacagagcctctcagatcttcagcggtcccgccgtcgtcgcagaagagacctggtatggactagagcaggagtacactctactccagaaggacgtgaagtggcctctgggctggcctctgggcggcttccctgctccacagggtccgtactactgcggtataggcgtggacaaggcgttcgggagagagatcgtcgacgcccactacaaggcctgcctgtacgcaggaatcaacatcagcgggatcaatggcgaagtcatgcctggacagtgggagttccaagttggaccatccgtcggaatctcagcctccgatcagctctggatcgctcgctacctcttggagaggatcacagaggtcgccggagttgttctctccttgcaccccaagccaatcaagggtgactggaacggcgctggatgccacaccaactacagtaccaaatcgatgagggaggatggcggatacgagctgatcaagaaggcgatcgacaagctcggactcaggcacaaggaacacatcgaggcctacggcgaggataacgaggagcgtctcactggccgccacgagaccgccgacatccacaccttcaaatggggcgtggccaaccggggagcttcgatccgcgtcggacgggacacggagaaggaaggaaaaggttacttcgaggacaggaggccggcttccaacatggacccgtacgtggtgacctccatgatcgcggagacgacccttctctggaagccctga

SEQ ID NO: 9 - protein, 356 aa, Lemna minor
MALLADLQNLNLTETTEKIIAEYIWIGGSGLDMRSKARTISKPVSDPKELPKWNYDGSSTGQAPGEDSEVILYPQAIFRDPFRKGNNILVMCDCYTPAGEPIPTNKRYRASQIFSGPAVVAEETWYGLEQEYTLLQKDVKWPLGWPLGGFPAPQGPYYCGIGVDKAFGREIVDAHYKACLYAGINISGINGEVMPGQWEFQVGPSVGISASDQLWIARYLLERITEVAGVVLSLHPKPIKGDWNGAGCHTNYSTKSMREDGGYELIKKAIDKLGLRHKEHIEAYGEDNEERLTGRHETADIHTFKWGVANRGASIRVGRDTEKEGKGYFEDRRPASNMDPYVVTSMIAETTLLWKP

SEQ ID NO: 10 - DNA, 1233 bp, Lemna minor
  5′UTR (1)..(33): 5′UTR for glutamine synthetase 1 (GS1) isoform #2
caatttcctctgctcccgcttctgatctctgcaatggctcttctcaccgatctccagaacctgaatctcaccgagaccacggagaagatcatcgccgagtacatatggatcggcggctctggcttggacatgaggagcaaggcgaggacgatctccaaaccggtgtctgatcccaaggaactccccaagtggaactacgacggctccagcacaggtcaagctccaggagaggacagcgaagttatcctctatccccaggccatcttcagggatccattcaggaagggaaacaacattctcgtgatgtgcgactgctacacgccagcgggagagccgatccccacgaacaagaggtacagagcctctcagatcttcagcgatcccgccgtcgtcgcagaagagacctggtatggactagagcaggagtacactctcctccagaaggacgtgaaatggcctctgggctggcctctgggaggcttccctgctccacagggtccgtactactgcggtataggtgtggacaaggcgttcgggagggagatcgtcgacgcccactacaaggcctgcctgtacgctggaatcaacatcagcgggatcaatggcgaagtcatgcctggacagtgggagttccaagttggaccagccgtcggaatctccgcctccgatcagctctgggtcgctcgctacctcttggagaggatcacagaggttgccggagttgttctctccttgcaccccaagccaatcaagggtgactggaacggcgctggatgccacaccaactacagtaccaaatcgatgagggaggaaggcggatacgagctgatcaagaaggcgatcgacaaactcggactgaggcacaaggagcacatcggggcctacggtgaagacaacgaagagcgtctcaccggccgccacgagaccgccgacatccacaccttcaaatggggcgtggccaaccggggggcttcaatccgcgccggaagggacacggagaaggaaggaaaaggttacttcgaggacaggaggccggcttccaacatggacccgtacgtggtgacctccatggtcgcggagacgacccttctctggaagccctgatcgcggagcttcttccatggtcgtccagttcgtcctctttcaatttctgtcaaaatggctggctttttcatccttcctggatgtgggtctgtgtcggctgggtgagtgattggttgacttctccccctc

SEQ ID NO: 11 - DNA, 1071 bp, Lemna minor
  CDS (1)..(1071): Encodes glutamine synthetase 1 (GS1) isoform #2
atggctcttctcaccgatctccagaacctgaatctcaccgagaccacggagaagatcatcgccgagtacatatggatcggcggctctggcttggacatgaggagcaaggcgaggacgatctccaaaccggtgtctgatcccaaggaactccccaagtggaactacgacggctccagcacaggtcaagctccaggagaggacagcgaagttatcctctatccccaggccatcttcagggatccattcaggaagggaaacaacattctcgtgatgtgcgactgctacacgccagcgggagagccgatccccacgaacaagaggtacagagcctctcagatcttcagcgatcccgccgtcgtcgcagaagagacctggtatggactagagcaggagtacactctcctccagaaggacgtgaaatggcctctgggctggcctctgggaggcttccctgctccacagggtccgtactactgcggtataggtgtggacaaggcgttcgggagggagatcgtcgacgcccactacaaggcctgcctgtacgctggaatcaacatcagcgggatcaatggcgaagtcatgcctggacagtgggagttccaagttggaccagccgtcggaatctccgcctccgatcagctctgggtcgctcgctacctcttggagaggatcacagaggttgccggagttgttctctccttgcaccccaagccaatcaagggtgactggaacggcgctggatgccacaccaactacagtaccaaatcgatgagggaggaaggcggatacgagctgatcaagaaggcgatcgacaaactcggactgaggcacaaggagcacatcggggcctacggtgaagacaacgaagagcgtctcaccggccgccacgagaccgccgacatccacaccttcaaatggggcgtggccaaccggggggcttcaatccgcgccggaagggacacggagaaggaaggaaaaggttacttcgaggacaggaggccggcttccaacatggacccgtacgtggtgacctccatggtcgcggagacgacccttctctggaagccctga

SEQ ID NO: 12 - protein, 356 aa, Lemna minor
MALLTDLQNLNLTETTEKIIAEYIWIGGSGLDMRSKARTISKPVSDPKELPKWNYDGSSTGQAPGEDSEVILYPQAIFRDPFRKGNNILVMCDCYTPAGEPIPTNKRYRASQIFSDPAVVAEETWYGLEQEYTLLQKDVKWPLGWPLGGFPAPQGPYYCGIGVDKAFGREIVDAHYKACLYAGINISGINGEVMPGQWEFQVGPAVGISASDQLWVARYLLERITEVAGVVLSLHPKPIKGDWNGAGCHTNYSTKSMREEGGYELIKKAIDKLGLRHKEHIGAYGEDNEERLTGRHETADIHTFKWGVANRGASIRAGRDTEKEGKGYFEDRRPASNMDPYVVTSMVAETTLLWKP

SEQ ID NO: 13 - DNA, 1551 bp, Lemna minor
  5′UTR (1)..(204): 5′UTR for glutamine synthetase 2 (GS2) isoform #1
agcgagtaagctgccgtattctgcatgcgtggaccagattgatcttagcccctctcttttatcgactctaaacaattcacacacatattctctctccccccctttctctctaaatcttctctcctcttcaccgacgccgcagccggaggatccacattattctgtgtcgtccttgctcggagtttctcgagcggaggaaaaaagatggcggcgcagattcccgctccatcgctgcgatgcgagaggagcatcgcgatcaggccatcgctggcgcggaatcctctgatgcttgctcagagaggctcgccggcgtccagaaaaggaggacctgtcagatacagaggcttctccgtgcgcgcggtgctaggcaaccggaacaacgccgtctcgaggctggaggatcttctcaacctcgatctcaacccccacactgagaagatcatcgcggagtacatctggattggcggatcaggaatcgatgtacgcagcaaatcaaggaccatctccagaccagtggatgatccttctgagctacccaagtggaattacgacggatctagcactggacaagctccaggagaagacagtgaagttatcctctaccctcaagcaattttcaaggatcctttccgaggagggaacaacatcttggttatgtgcgatgcttacaaaccaaatggagagccgatccccacgaataaacggtacagggctgctcagatcttcagtgacccaaaggttgttgccgaagtcccatggtttggaattgaacaagagtacactttgctccagccaaatgtgaattggcctcttggctggcctattggaggatatcccggtcctcagggtccctactattgttcagctggtgcggagaagtcgtttgggcgtgatatatcagacgcccactacaaagcatgcctatatgctgggattaacattagtggtactaatgcagaagttatgcctggccagtgggaatatcaagtgggcccaagcgttggtattgatgccggtgatcatatctgggtttctagatacattctggagagaatcacggaacaagccggagttgtcctctccctcgatcctaaacccatcgagggtgactggaacggcgctggatgccacaccaattatagtacaaagacaatgagagaggagggaggattcgaggtgattaagaaggctgtggtcaatctctcccttcgtcacaaggagcatatcagcgcatatggagaaggaaatgagcggcggttgacgggaaaacacgagaccgccaacatcaataccttctcttggggtgttgccaaccgtggttgctccgtgcgcgtgggtcgtgagaccgagaaggaaggcaaaggatacatggaagatcgccgccccgcatccaacatggatccatacgtggtgacatcacttcttgccgagacgacgatcctctgggagccttctgtggagttggttgcctcctcctaatgatgaagaagcatccatcatcatcgtcatcatctttcttctctcttgatctgcccataacgagatgaggag

SEQ ID NO: 14 - DNA, 1275 bp, Lemna minor
  CDS (1)..(1275): Encodes glutamine synthetase 2 (GS2) isoform #1
atggcggcgcagattcccgctccatcgctgcgatgcgagaggagcatcgcgatcaggccatcgctggcgcggaatcctctgatgcttgctcagagaggctcgccggcgtccagaaaaggaggacctgtcagatacagaggcttctccgtgcgcgcggtgctaggcaaccggaacaacgccgtctcgaggctggaggatcttctcaacctcgatctcaacccccacactgagaagatcatcgcggagtacatctggattggcggatcaggaatcgatgtacgcagcaaatcaaggaccatctccagaccagtggatgatccttctgagctacccaagtggaattacgacggatctagcactggacaagctccaggagaagacagtgaagttatcctctaccctcaagcaattttcaaggatcctttccgaggagggaacaacatcttggttatgtgcgatgcttacaaaccaaatggagagccgatccccacgaataaacggtacagggctgctcagatcttcagtgacccaaaggttgttgccgaagtcccatggtttggaattgaacaagagtacactttgctccagccaaatgtgaattggcctcttggctggcctattggaggatatcccggtcctcagggtccctactattgttcagctggtgcggagaagtcgtttgggcgtgatatatcagacgcccactacaaagcatgcctatatgctgggattaacattagtggtactaatgcagaagttatgcctggccagtgggaatatcaagtgggcccaagcgttggtattgatgccggtgatcatatctgggtttctagatacattctggagagaatcacggaacaagccggagttgtcctctccctcgatcctaaacccatcgagggtgactggaacggcgctggatgccacaccaattatagtacaaagacaatgagagaggagggaggattcgaggtgattaagaaggctgtggtcaatctctcccttcgtcacaaggagcatatcagcgcatatggagaaggaaatgagcggcggttgacgggaaaacacgagaccgccaacatcaataccttctcttggggtgttgccaaccgtggttgctccgtgcgcgtgggtcgtgagaccgagaaggaaggcaaaggatacatggaagatcgccgccccgcatccaacatggatccatacgtggtgacatcacttcttgccgagacgacgatcctctgggagccttctgtggagttggttgcctcctcctaa

SEQ ID NO: 15 - protein, 424 aa, Lemna minor
MAAQIPAPSLRCERSIAIRPSLARNPLMLAQRGSPASRKGGPVRYRGFSVRAVLGNRNNAVSRLEDLLNLDLNPHTEKIIAEYIWIGGSGIDVRSKSRTISRPVDDPSELPKWNYDGSSTGQAPGEDSEVILYPQAIFKDPFRGGNNILVMCDAYKPNGEPIPTNKRYRAAQIFSDPKVVAEVPWFGIEQEYTLLQPNVNWPLGWPIGGYPGPQGPYYCSAGAEKSFGRDISDAHYKACLYAGINISGTNAEVMPGQWEYQVGPSVGIDAGDHIWVSRYILERITEQAGVVLSLDPKPIEGDWNGAGCHTNYSTKTMREEGGFEVIKKAVVNLSLRHKEHISAYGEGNERRLTGKHETANINTFSWGVANRGCSVRVGRETEKEGKGYMEDRRPASNMDPYVVTSLLAETTILWEPSVELVASS

SEQ ID NO: 16 - DNA, 1551 bp, Lemna minor
  5′UTR (1)..(204): 5′UTR for glutamine synthetase 2 (GS2) isoform #2
agcgagtaagctgccgtattctgcatgcgtggaccagattgatcttagcccctctcttttatcgactctaaacaattcacacacatattctctctccccccctttctctctaaatcttctctcctcttcaccgacgccgcagccggaggatccacattattctgtgtcgtccttgctcggagtttctcgagcggaggaaaaaagatggcggcgcagattcccgctccatcgctgcgatgcgagaggagcatcgcgatcaggccatcgctggcgcggaatcctctgatgcttgctcagagaggctcgccggcgtccagaaaaggaggacctgtcagatacagaggcttctccgtgcgcgcggtgctaggcaaccggaacaacgccgtctcgaggctggaggatcttctcaacctcgatctcaacccccacactgagaagatcatcgcggagtacatctggattggcggatcaggaatcgatgtacgcagcaaatcaaggaccatctccagaccagtggatgatccttctgagctacccaagtggaattacgacggatctagcactggacaagctccaggagaagacagcgaagttatcctctaccctcaagcaattttcaaggatcctttccgaggagggaacaacatcttggttatgtgtgatgcttacaaaccaaatggagagccgatccccacgaataaacggtacagggctgctcagatctttagtgacccaaaggttgttgccgaagtcccatggtttggaattgaacaagaatacactttgctccagccgaatgtgaattggcctcttggctggcctattggaggatatcctggacctcagggtccctactattgttcagctggtgcggataagtcgtttgggcgtgatatatcagacgcccactacaaagcgtgcctatatgctgggattaacattagtggtactaatgcagaagttatgcctggccagtgggaatatcaagtgggcccaagcgctggaattgatgctggagatcatatctgggtctctagatacattctggagagaatcacggagcaagccggagttgtgctctccctcgatcctaaacccatcgagggtgactggaacggcgctggatgccacaccaattacagtacaaagacaatgagagaggatggaggattcgaggagattaagaaggctgtggtcaatctctctcttcgtcacaaggagcatattagcgcgtatggagaaggaaacgagcggcggttgacgggaaaacacgagaccgccaacatcaataccttctcttggggtgttgccaaccgtggttgctctgtgcgcgtgggtcgtgagaccgagaaggaaggcaaaggatacatggaagatcgccgccccgcatccaacatggatccatacgtggtgacatcacttcttgccgagacgacgatcctctgggagccttctgtggagttggttgcctcctcctaatgatgaagaagcatccatcatcgtcgtcatcatctttcttctctcttgatctgcccataacgagatgaggag

SEQ ID NO: 17 - DNA, 1275 bp, Lemna minor
  CDS (1)..(1275): Encodes glutamine synthetase 2 (GS2) isoform #2
atggcggcgcagattcccgctccatcgctgcgatgcgagaggagcatcgcgatcaggccatcgctggcgcggaatcctctgatgcttgctcagagaggctcgccggcgtccagaaaaggaggacctgtcagatacagaggcttctccgtgcgcgcggtgctaggcaaccggaacaacgccgtctcgaggctggaggatcttctcaacctcgatctcaacccccacactgagaagatcatcgcggagtacatctggattggcggatcaggaatcgatgtacgcagcaaatcaaggaccatctccagaccagtggatgatccttctgagctacccaagtggaattacgacggatctagcactggacaagctccaggagaagacagcgaagttatcctctaccctcaagcaattttcaaggatcctttccgaggagggaacaacatcttggttatgtgtgatgcttacaaaccaaatggagagccgatccccacgaataaacggtacagggctgctcagatctttagtgacccaaaggttgttgccgaagtcccatggtttggaattgaacaagaatacactttgctccagccgaatgtgaattggcctcttggctggcctattggaggatatcctggacctcagggtccctactattgttcagctggtgcggataagtcgtttgggcgtgatatatcagacgcccactacaaagcgtgcctatatgctgggattaacattagtggtactaatgcagaagttatgcctggccagtgggaatatcaagtgggcccaagcgctggaattgatgctggagatcatatctgggtctctagatacattctggagagaatcacggagcaagccggagttgtgctctccctcgatcctaaacccatcgagggtgactggaacggcgctggatgccacaccaattacagtacaaagacaatgagagaggatggaggattcgaggagattaagaaggctgtggtcaatctctctcttcgtcacaaggagcatattagcgcgtatggagaaggaaacgagcggcggttgacgggaaaacacgagaccgccaacatcaataccttctcttggggtgttgccaaccgtggttgctctgtgcgcgtgggtcgtgagaccgagaaggaaggcaaaggatacatggaagatcgccgccccgcatccaacatggatccatacgtggtgacatcacttcttgccgagacgacgatcctctgggagccttctgtggagttggttgcctcctcctaa

SEQ ID NO: 18 - protein, 424 aa, Lemna minor
MAAQIPAPSLRCERSIAIRPSLARNPLMLAQRGSPASRKGGPVRYRGFSVRAVLGNRNNAVSRLEDLLNLDLNPHTEKIIAEYIWIGGSGIDVRSKSRTISRPVDDPSELPKWNYDGSSTGQAPGEDSEVILYPQAIFKDPFRGGNNILVMCDAYKPNGEPIPTNKRYRAAQIFSDPKVVAEVPWFGIEQEYTLLQPNVNWPLGWPIGGYPGPQGPYYCSAGADKSFGRDISDAHYKACLYAGINISGTNAEVMPGQWEYQVGPSAGIDAGDHIWVSRYILERITEQAGVVLSLDPKPIEGDWNGAGCHTNYSTKTMREDGGFEEIKKAVVNLSLRHKEHISAYGEGNERRLTGKHETANINTFSWGVANRGCSVRVGRETEKEGKGYMEDRRPASNMDPYVVTSLLAETTILWEPSVELVASS

SEQ ID NO: 19 - DNA, 1266 bp, Lemna minor
  5′UTR (1)..(53): 5′UTR for biotin synthase (BS) isoform #1
ctgccattagcgtggggagatacgcggagagatcggaggcagaggattagaagatgctgttgatccggtctctccgagcgcgagtccatcgctcgtcctcgagcttcgccttctccacggctgccgcatcggcggcgactgtgcaggcggaacgaacgataagggatgggccgaggactgattggagcaaggacgaggtcaaagcggtttacgattctcccgtcctcgatctccttttccatggcgcccaagtccacaggcacgtgcacaagttcagggaagtgcaacagtgtactcttctctccatcaagacaggtgggtgcagcgaagattattcatattgcccgcaatcgtctcgctatgatacggggttgaaagctcaaaggctcatgaccaaggatgatgttctggaagcagcaaaaaaggcaaaagatgctggcagcacacgtttctgcatgggggctgcatggcgggatacaattggccggaaaaccaacttcaaccagattctcaattacgtcaaagaaattagggagatgggcatggaggtgtgttgcactctaggcatgctagagaagcagcaagctgaggagcttaagaaagcagggcttacggcgtataatcacaatcttgacacttcaagagagtattatcccaacattataaccacaagatcatttgatgaacggctggaaaccctccaacatgttcgtgaggcaggaataagtgtctgctccggtggaataattgggctgggtgaagcagaagaagaccgggttggactcctgcacactctagccaccctccctactcatccagagagcgtacccattaatgcactcgtaccagttaagggcactcccctccaagatcaaaagcctgtggagatctgggagatgatcaggatgaccgcaacggcgcgcatcgtgatgccacaagcaatggtgcggctctcagcaggccgagttcgcttctccatgcccgagcaggccctttgcttcctcgcaggcgccaactccatcttcaccggagaaaagcttctcaccactgccaacaacgactttgacgcagatcagctcatgttcaaagttctcggtctcattcccaaggcacctagcttttctgaggagtttcaggagacggcggcagagagccctgagctggcggcggtttcaagttccggttgaattctccgagctagcattaagtatttgaacctcagaacaaaggcggtaattagtacttgaggtgagcttatatgaggga

SEQ ID NO: 20 - DNA, 1134 bp, Lemna minor
  CDS (1)..(1134): Encodes biotin synthase (BS) isoform #1
atgctgttgatccggtctctccgagcgcgagtccatcgctcgtcctcgagcttcgccttctccacggctgccgcatcggcggcgactgtgcaggcggaacgaacgataagggatgggccgaggactgattggagcaaggacgaggtcaaagcggtttacgattctcccgtcctcgatctccttttccatggcgcccaagtccacaggcacgtgcacaagttcagggaagtgcaacagtgtactcttctctccatcaagacaggtgggtgcagcgaagattattcatattgcccgcaatcgtctcgctatgatacggggttgaaagctcaaaggctcatgaccaaggatgatgttctggaagcagcaaaaaaggcaaaagatgctggcagcacacgtttctgcatgggggctgcatggcgggatacaattggccggaaaaccaacttcaaccagattctcaattacgtcaaagaaattagggagatgggcatggaggtgtgttgcactctaggcatgctagagaagcagcaagctgaggagcttaagaaagcagggcttacggcgtataatcacaatcttgacacttcaagagagtattatcccaacattataaccacaagatcatttgatgaacggctggaaaccctccaacatgttcgtgaggcaggaataagtgtctgctccggtggaataattgggctgggtgaagcagaagaagaccgggttggactcctgcacactctagccaccctccctactcatccagagagcgtacccattaatgcactcgtaccagttaagggcactcccctccaagatcaaaagcctgtggagatctgggagatgatcaggatgaccgcaacggcgcgcatcgtgatgccacaagcaatggtgcggctctcagcaggccgagttcgcttctccatgcccgagcaggccctttgcttcctcgcaggcgccaactccatcttcaccggagaaaagcttctcaccactgccaacaacgactttgacgcagatcagctcatgttcaaagttctcggtctcattcccaaggcacctagcttttctgaggagtttcaggagacggcggcagagagccctgagctggcggcggtttcaagttccggttga

SEQ ID NO: 21 - protein, 377 aa, Lemna minor
MLLIRSLRARVHRSSSSFAFSTAAASAATVQAERTIRDGPRTDWSKDEVKAVYDSPVLDLLFHGAQVHRHVHKFREVQQCTLLSIKTGGCSEDYSYCPQSSRYDTGLKAQRLMTKDDVLEAAKKAKDAGSTRFCMGAAWRDTIGRKTNFNQILNYVKEIREMGMEVCCTLGMLEKQQAEELKKAGLTAYNHNLDTSREYYPNIITTRSFDERLETLQHVREAGISVCSGGIIGLGEAEEDRVGLLHTLATLPTHPESVPINALVPVKGTPLQDQKPVEIWEMIRMTATARIVMPQAMVRLSAGRVRFSMPEQALCFLAGANSIFTGEKLLTTANNDFDADQLMFKVLGLIPKAPSFSEEFQETAAESPELAAVSSSG

SEQ ID NO: 22 - DNA, 1266 bp, Lemna minor
  5′UTR (1)..(53): 5′UTR for biotin synthase (BS) isoform #2
ctgccattagcgtggggagatacgcggagagatcggaggcagaggattagaagatgctgttgatccggtctctccgagcgcgagtccatcgctcgtcctcgagcttcgccttctccacggctgccgcatcggcggcgactgtgcaggcggaacgaacgataagggatgggccgaggactgattggagcaaggacgaggtcaaagcggtttacgattctcccgtcctcgatctccttttccatggcgcccaagtccacaggcacgtgcacaagttcagggaagtgcaacagtgtactcttctctccatcaagacaggtgggtgcagcgaagattgttcatattgcccgcaatcgtctcgctatgatacggggttgaaagctcaaaggctcatgaccaaggatgatgttctggaagcagcaaaaaaggcaaaagatgctggcagcacacgtttctgcatgggggctgcatggcgggatacaattggccggaaaaccaacttcaaccagattctcaattacgtcaaagaaattagggagatgggcatggaggtgtgttgcactctaggcatgctagagaagcagcaagctgaggagcttaagaaagcagggcttacggcgtataatcacaatcttgacacttcaagagagtattatcccaacattataaccacaagatcatttgatgaacggctggaaaccctccaacatgttcgtgaggcaggaataagtgtctgctcaggtggaataattgggctgggtgaagcagaagaagaccgggttggactcctgcacactctagccaccctccctactcatccagagagcgtacccattaatgcactcgtaccagttaagggcactcccctccaagatcaaaagcctgtggagatctgggagatgatcaggatgatcgcaacggcgcgcatcgtgatgccacaagcaatggtgcggctctcagcaggccgagttcgcttctccatgcccgagcaggccctttgcttcctcgcaggcgccaactccatcttcaccggagaaaagcttctcaccactgccaacaacgactttgacgcagatcagctcatgttcaaagttctcggtctcattcccaaggcacctagcttttctgaggagtttcaggagacggcggcagagagccctgagctggcggcggtttcaagttccggttgaattctccgagctagcattaagtatttgagcctcagaacaaaggcggtaattagtacttgaggtgagcttatatgaggga

SEQ ID NO: 23 - DNA, 1134 bp, Lemna minor
  CDS (1)..(1134): Encodes biotin synthase (BS) isoform #2
atgctgttgatccggtctctccgagcgcgagtccatcgctcgtcctcgagcttcgccttctccacggctgccgcatcggcggcgactgtgcaggcggaacgaacgataagggatgggccgaggactgattggagcaaggacgaggtcaaagcggtttacgattctcccgtcctcgatctccttttccatggcgcccaagtccacaggcacgtgcacaagttcagggaagtgcaacagtgtactcttctctccatcaagacaggtgggtgcagcgaagattgttcatattgcccgcaatcgtctcgctatgatacggggttgaaagctcaaaggctcatgaccaaggatgatgttctggaagcagcaaaaaaggcaaaagatgctggcagcacacgtttctgcatgggggctgcatggcgggatacaattggccggaaaaccaacttcaaccagattctcaattacgtcaaagaaattagggagatgggcatggaggtgtgttgcactctaggcatgctagagaagcagcaagctgaggagcttaagaaagcagggcttacggcgtataatcacaatcttgacacttcaagagagtattatcccaacattataaccacaagatcatttgatgaacggctggaaaccctccaacatgttcgtgaggcaggaataagtgtctgctcaggtggaataattgggctgggtgaagcagaagaagaccgggttggactcctgcacactctagccaccctccctactcatccagagagcgtacccattaatgcactcgtaccagttaagggcactcccctccaagatcaaaagcctgtggagatctgggagatgatcaggatgatcgcaacggcgcgcatcgtgatgccacaagcaatggtgcggctctcagcaggccgagttcgcttctccatgcccgagcaggccctttgcttcctcgcaggcgccaactccatcttcaccggagaaaagcttctcaccactgccaacaacgactttgacgcagatcagctcatgttcaaagttctcggtctcattcccaaggcacctagcttttctgaggagtttcaggagacggcggcagagagccctgagctggcggcggtttcaagttccggttga

SEQ ID NO: 24 - protein, 377 aa, Lemna minor
MLLIRSLRARVHRSSSSFAFSTAAASAATVQAERTIRDGPRTDWSKDEVKAVYDSPVLDLLFHGAQVHRHVHKFREVQQCTLLSIKTGGCSEDCSYCPQSSRYDTGLKAQRLMTKDDVLEAAKKAKDAGSTRFCMGAAWRDTIGRKTNFNQILNYVKEIREMGMEVCCTLGMLEKQQAEELKKAGLTAYNHNLDTSREYYPNIITTRSFDERLETLQHVREAGISVCSGGIIGLGEAEEDRVGLLHTLATLPTHPESVPINALVPVKGTPLQDQKPVEIWEMIRMIATARIVMPQAMVRLSAGRVRFSMPEQALCFLAGANSIFTGEKLLTTANNDFDADQLMFKVLGLIPKAPSFSEEFQETAAESPELAAVSSSG

SEQ ID NO: 25 - DNA, 27 bp, Artificial Sequence
  Primer
gcagcccgtgttctccttyaarytnmg

SEQ ID NO: 26 - DNA, 26 bp, Artificial Sequence
  Primer
tggaagagggwgatgttccanykngg

SEQ ID NO: 27 - DNA, 22 bp, Artificial Sequence
  Primer
ccgccggcaaccaygcncargg

SEQ ID NO: 28 - DNA, 30 bp, Artificial Sequence
  Primer
gtgcagcttgtcgaagtycatrttngcncc

SEQ ID NO: 29 - DNA, 19 bp, Artificial Sequence
  Primer
ctcggcataggcgataagt

SEQ ID NO: 30 - DNA, 19 bp, Artificial Sequence
  Primer
gaggcccgattcatgccat

SEQ ID NO: 31 - DNA, 18 bp, Artificial Sequence
  Primer
gcggaatgaaagttcggc

SEQ ID NO: 32 - DNA, 18 bp, Artificial Sequence
  Primer
agtatcctcgagccagcc

SEQ ID NO: 33 - DNA, 22 bp, Artificial Sequence
  Primer
ctctcggatcctgcatcgtctt

SEQ ID NO: 34 - DNA, 23 bp, Artificial Sequence
  Primer
cagaagccataacaccgcataca

SEQ ID NO: 35 - DNA, 28 bp, Artificial Sequence
  Primer
tatgtcgacatgaaggtcaccaccgact

SEQ ID NO: 36 - DNA, 27 bp, Artificial Sequence
  Primer
ttctagacaaaattttcaaaccccatg

SEQ ID NO: 37 - DNA, 27 bp, Artificial Sequence
  Primer
ttctagacgccatggcatttgcatcgt

SEQ ID NO: 38 - DNA, 27 bp, Artificial Sequence
  Primer
tgagctcatgaaggtcaccaccgactc

SEQ ID NO: 39 - DNA, 23 bp, Artificial Sequence
  Primer
tgccctagagatgtccaacaagg

SEQ ID NO: 40 - DNA, 2011 bp, Spirodela polyrrhiza
gatggacagataatgagatgaattagaaaaaaaaaattcgtgttgtaagatagaatacttgctatctactgatgaatgcagttcagttttcctcacgatcttaaagatcgcgcactatcctcagcttcactctggaaattttgattctcttcttctgctcagcagcctcgactctgtctagggtttcgtacaatcggacgccattctacatgaatcgagcacagggaatgaagacaattaggagatcctcgatgtcctccgacttacttgcatgacttgacggggaagatctcgagcagggaagcgacgcctctccggaggactcgcctcgccgagaggacctcctccgcgacacggaccatggcctccacggggtagaagctggccctgttctttattctcttgaggatcatcggccgaagcctccgcaaatccatccccgaggagtagaatctcgcctgcaggaagcatctgtcgagatcctcgccgaggcggcggagatacctcgccggcgccgccatggcgccggggacggagcaccaccacggagaagaagaaccctaacccaaggcattaacgaagttgcgcagattatacaaaagccctcaaatatctttcattttctatttcactgatacattttcattattgtatatgagtgtttatttaaattattccgtattagaaaagcacctccagaacccgacaaaatagggtgacgtcatcatggtgtcatgaccgcccaacagccgcagatttaaaatcggtggatgagtgcggccacgccacgaaagcgatgggccttcgtcgatgccgtgagaatccatctgacataaagtaaacggcgccgtcagtattgacggcgtatgacacgtggaaagaagctattggttcacgcatcggtggttccgctagcctccgtcgaccgctagtactataaatacggtcccgaggcctcctcaccactcgcacatatcctctttgttttcctctccgtgaaagaagcgaggaagcgcgtcgtctctcccaaggtaaggagcagatctctttgatcgtttttgttcttcttttgttttgttttttttttctgcggatcttcggttgcatcatgccttggctgtttttattagtttaggatatcctcgtttggatctgagccgatcatatatgttaaaggttgtgttcgatctctttgttcattttcgcatgaaaaggatgtatccttttgatgtgaggcgatcttctatggttaagactttgttcggtctattgatcatttctgttcttcgtttttgagtttttttctgcggatatcgcatcatccctaggtttttgctttggttaggatgcatcctttggatttgagccgatctcccttggttaaggctgtgtctgttgcagaggagaaagtctgtcgaggtccttatgcaggctttgtccagatgcgcgtgctctctcatgctatgaatttatgttttgagaactcctcccggtttttctagatccggatttgaagtattcattgcggttccccttcggttttatgtatttctcgagttgatttggtccatgatcgtgttctgtccagatctctcttgatatggatgagatattcgttacctctttcaaacatcggtggatgttctttttagtcttggctcacctttatctagaaattaattttcggtttgaaacccctgcttgttaaggtgatgtattccttctttatagatttcggtgtgttatttcttaacggtgatctgtccgatccatgtgttgcacctcttgttttctgtgtaatcctctgtgaattataattatgttttgaaaacgtacttaagtaaggggcatgttccccgtttaaaacttttgttctatcaatttgtggttaatagatcctgatttgtggtcgccttattctgtctttaatcgtggattttatttatcttgagcgcgtccttttcttttaaaatcatgtgtttaacctttcagtcgtcatatgttccatcag

SEQ ID NO: 41 - DNA, 1142 bp, Artificial Sequence
  Truncated SpUbq117 promoter
acacgtggaaagaagctattggttcacgcatcggtggttccgctagcctccgtccaccgctagtactataaatacggtcccgaggcctcctcaccactcgcacatatcctctttgttttcctctccgtgaaagaagcgaggaagcgcgtcgtctctcccaaggtaaggagcagatctctttgatcgtttttgttcttcttttgttttgttttttttttctgcggatcttcggttgcatcatgccttggctgtttttattagtttaggatatcctcgtttggatctgagccgatcatatatgttaaaggttgtgttcgatctctttgttcattttcgcrtgaaaaggatgtatccttttgatgtgaggcgatcttctatggttaagactttgttcggtctattgatcatttctgttcttcgtttttgagtttttttctgcggatatcgcatcatccctaggtttttgctttggttaggatgcatcctttggatttgagccgatctcccttggttaaggctgtgtctgttgcagaggagaaagtctgtcgaggtccttatgcaggctttgtccagatgcgcgtgctctctcatgctatgaatttatgttttgagaactcctcccggtttttctagatccggatttgaagtattcattgcggttccccttcggttttatgtatttctcgagttgatttggtccatgatcgtgttctgtccagatctctcttgatatggatgagatattcgttacctctttcaaacatcggtggatgttctttttagtcttggctcacctttatctagaaattaattttcggtttgaaacccctgcttgttaaggtgatgtattccttctttatagatttcggtgtgttatttcttaacggtgatctgtccgatccatgtgttgcacctcttgttttctgtgtaatcctctgtgaattataattatgttttgaaaacgtacttaagtaaggggcatgttccccgtttaaaacttttgttctatcaatttgtggttaatagatcctgatttgtggtcgccttattctgtctttaatcgtggattttatttatcttgagcgcgtccttttcttttaaaatcatgtgtttaacctttcagtcgtcatatgttccatcag

(19) United States
(12) Patent Application Publication
(10) Pub. No.: US 2014/0216118 A1
(43) Pub. Date: Aug. 7, 2014

(54) Compositions and Methods for Making and Biocontaining Auxotrophic Transgenic Plants

(71) Applicants: Kevin M. Cox, Raleigh, NC (US); Long Nguyen, Apex, NC (US)

(72) Inventors: Kevin M. Cox, Raleigh, NC (US); Long Nguyen, Apex, NC (US)

(73) Assignee: SYNTHON BIOPHARMACEUTICALS B.V., Nijmegen (NL)

(21) Appl. No.: 14/126,427

(22) PCT Filed: Jun. 13, 2012
(86) PCT No.: PCT/US2012/042286

§ 371 Date: Apr. 16, 2014

Related U.S. Application Data
(60) Provisional application No. 61/496,864, filed on Jun. 14, 2011.

(51) Int. Cl. C12N 15/82

(52) U.S. Cl. CPC C12N 15/8238
USPC 71/27; 800/298; 435/419

(57) ABSTRACT

Compositions and methods are described for making and using transgenic plants and plant parts having at least one auxotrophic requirement for an essential compound such as an amino acid, carbohydrate, fatty acid, nucleic acid, vitamin, plant hormone, or precursor thereof. Transgenic plants and plants parts having at least one auxotrophic requirement can be effectively biocontained by withdrawal of the essential compound.

### REFERENCE TO SEQUENCE LISTING SUBMITTED ELECTRONICALLY

An official copy of a Sequence Listing submitted electronically via EFS-Web as an ASCII formatted Sequence Listing with a file named “420183SEQLIST.txt,” created on Jun. 13, 2012, and having a size of 125 KB and filed concurrently with the Specification is a part of the Specification and is incorporated herein by reference as if set forth in its entirety.

### FIELD OF THE INVENTION

The present invention relates to transgenic plants and plant parts, particularly transgenic plants and plant parts having an auxotrophic requirement.

### BACKGROUND

Movement of genes among plant species, often called horizontal (or lateral) gene transfer, can occur by natural processes or recombinant DNA technologies such as transformation. Transgenic plants made by recombinant DNA technologies are deliberately developed for a variety of reasons including disease resistance, herbicide resistance, pest resistance, non-biological stress resistance such as to drought or nitrogen starvation, nutritional improvement, and recombinant protein production.

A concern with transgenic plants, however, is their impact outside a laboratory on biodiversity and ecosystems. Because transgenes can flow by vertical and/or horizontal gene transfer, they have a potential for significant ecological impact if they increase in frequency and enter conventional crops or wild-type populations. Likewise, transfer of genes from conventional crops or wild-type populations to transgenic plants also can be a concern.

Of interest herein is biological containment (or biocontainment, also referred to as biological confinement or bioconfinement) of transgenes present in transgenic plants and plant parts, particularly transgenic plants utilized for recombinant protein production. Biocontainment relates to measures that prevent transgenes from entering the genome of conventional crops or wild-type populations (i.e., non-genetically modified organisms). Strategies for biocontainment of transgenes can be based upon physical or biological barriers and include the prevention of the release of transgenic plant material from laboratory settings. Physical strategies for biocontainment include spatial barriers such as a zone of open land or other crops between transgenic plants and conventional crops or wild-type populations to confine cross-movement of pollen and seeds. Other physical strategies include temporal isolation, such as delayed planting and crop rotation, and covering flowers or detasseling.

Biological strategies for biocontainment include alloploidy. Other biological strategies include localizing a transgene to a subcellular organelle that is strictly maternally inherited (e.g., chloroplast or mitochondria), utilizing genetic use restriction technology (GURT or terminator technology; see, e.g., U.S. Pat. No. 5,723,765), engineering plants to be infertile or sterile, and engineering plants to be asexual. Dioecy and cleistogamy are still other biological strategies.

Biocontainment strategies, both from an engineering and biological point of view, are therefore necessary to prevent escape of transgenes to conventional crops or wild-type populations. For the foregoing reasons, there is a need for additional compositions and methods for biocontaining transgenes present in transgenic plants and plant parts.

### BRIEF SUMMARY

Compositions and methods are provided for making and using transgenic plants or plant parts that comprise a heterologous polynucleotide of interest and which have an auxotrophic requirement. Compositions of the invention include novel gene sequences and polynucleotide constructs for introducing an auxotrophic requirement into transgenic plants, as well as transgenic plants and plant parts having an auxotrophic requirement.

Methods of the invention include introducing an auxotrophic requirement into transgenic plants and plant parts, biocontaining transgenic plants and plant parts using this auxotrophic requirement, as well as production of recombinant polypeptides in transgenic plants and plant parts having an auxotrophic requirement.

The following embodiments are encompassed by the present invention.

1. A method for biocontaining a transgenic plant or plant part comprising a heterologous polynucleotide of interest, said method comprising:

providing an effective amount of an essential compound to said transgenic plant or plant part, wherein said transgenic plant or plant part has an auxotrophic requirement for said essential compound, and wherein said transgenic plant or plant part comprises a polynucleotide construct having a nucleotide sequence that inhibits expression or function of a component of a biosynthetic pathway for said essential compound, said nucleotide sequence being operably linked to a promoter that is functional in a plant cell, wherein said transgenic plant or plant part grows in the presence of said effective amount of said essential compound; and

removing said essential compound from said transgenic plant or plant part, wherein growth of said transgenic plant or plant part is inhibited in the absence of said compound, whereby said transgenic plant or plant part is biocontained.

2. The method of embodiment 1, wherein said essential compound is an amino acid, a carbohydrate, a fatty acid, a nucleic acid, a vitamin, a plant hormone, or a precursor thereof.

3. The method of embodiment 1 or embodiment 2, wherein said nucleotide sequence encodes an inhibitory nucleotide molecule that is capable of being transcribed as an inhibitory polynucleotide selected from the group consisting of a single-stranded RNA polynucleotide, a double-stranded RNA polynucleotide, and a combination thereof.

4. The method of embodiment 1 or embodiment 2, wherein said nucleotide sequence encodes a polypeptide that inhibits function of said component of said biosynthetic pathway.

5. The method of embodiment 4, wherein said polypeptide is an antibody or a binding protein that binds said component of the biosynthetic pathway for said essential compound, thereby inhibiting function of said component.

6. The method of any one of embodiments 1-5, wherein said promoter is a constitutive promoter.

7. The method of any one of embodiments 1-6, wherein said heterologous polynucleotide of interest encodes a heterologous polypeptide of interest, or wherein said heterologous polynucleotide of interest comprises a nucleotide sequence that inhibits expression or function of a target gene of interest, wherein said target gene of interest is other than a gene encoding for a component of a biosynthetic pathway for an essential compound.

8. The method of embodiment 7, wherein said heterologous polypeptide of interest is a mammalian polypeptide or biologically active variant thereof.

9. The method of embodiment 8, wherein the polypeptide of interest is selected from the group consisting of insulin, growth hormone, α-interferon, β-interferon, β-glucocerebrosidase, β-glucoronidase, retinoblastoma protein, p53 protein, angiostatin, leptin, erythropoietin (EPO), granulocyte macrophage colony stimulating factor, plasminogen, tissue plasminogen activator, blood coagulation factors, alpha 1-antitrypsin, a monoclonal antibody (mAbs), a Fab fragment, a single-chain antibody, cytokines, receptors, hormones, human vaccines, animal vaccines, peptides, and serum albumin.

10. The method of any one of embodiments 1-9, wherein said plant is a monocot.

11. The method of embodiment 10, wherein said monocot is a member of the Lemnaceae.

12. The method of embodiment 11, wherein said monocot is from a genus selected from the group consisting of the genus Spirodela, genus Wolfia, genus Wolflella, genus Landolttia, and genus Lemna.

13. The method of embodiment 12, wherein said monocot is a member of a species selected from the group consisting of Lemna minor, Lemna miniscula, Lemna aequinoctialls, and Lemna gibba.

14. The method of any one of embodiments 1-9, wherein said plant is a dicot.

15. A method for biocontaining a transgenic duckweed plant, plant cell, or nodule, wherein said transgenic duckweed plant, plant cell, or nodule comprises a heterologous polynucleotide of interest, said method comprising the steps of:

providing an effective amount of an essential compound to said transgenic duckweed plant, plant cell, or nodule, wherein said transgenic duckweed plant, plant cell, or nodule has an auxotrophic requirement for said essential compound, and

removing said essential compound from said transgenic duckweed plant, plant cell, or nodule, wherein growth of said transgenic duckweed plant, plant cell, or nodule is inhibited in the absence of said compound, whereby said transgenic duckweed plant, plant cell, or nodule is biocontained.

16. The method of embodiment 15, wherein the compound is an essential amino acid, a carbohydrate, a fatty acid, a nucleic acid, a vitamin, a plant hormone, or a precursor thereof.

17. The method of embodiment 15 or 16, wherein said auxotrophic requirement is introduced into said transgenic duckweed plant, plant cell, or nodule by a method selected from the group consisting of:
(a) expressing a polynucleotide or polypeptide in said transgenic duckweed plant, plant cell, or nodule, wherein said polynucleotide or polypeptide inhibits expression or function of a component of a biosynthetic pathway for said essential compound;
(b) eliminating a gene in said transgenic duckweed plant, plant cell, or nodule, wherein said gene encodes said component of said biosynthetic pathway for said essential compound; and
(c) mutating a gene in said transgenic duckweed plant, plant cell, or nodule, wherein said gene encodes said component of said biosynthetic pathway for said essential compound.

18. The method of embodiment 17, wherein said transgenic duckweed plant, plant cell, or nodule is stably transformed with a polynucleotide construct having a nucleotide sequence that is capable of inhibiting expression or function of said component of said biosynthetic pathway for said essential compound, said nucleotide sequence being operably linked to a promoter that is functional in a plant cell.

19. The method of embodiment 18, wherein said nucleotide sequence encodes a polypeptide that inhibits function of said component of said biosynthetic pathway.

20. The method of embodiment 19, wherein said polypeptide is an antibody or a binding protein that binds said component of the biosynthetic pathway for said essential compound, thereby inhibiting function of said component.

21. The method of embodiment 19 or 20, wherein said component of said biosynthetic pathway is biotin synthase.

22. The method of embodiment 21, wherein said nucleotide sequence encodes streptavidin or a fragment thereof that binds biotin synthase, thereby inhibiting function of said biotin synthase.

23. The method of embodiment 21 or embodiment 22, wherein said biotin synthase comprises an amino acid sequence having at least 90% sequence identity to the sequence set forth in SEQ ID NO:21 or SEQ ID NO:24.

24. The method of embodiment 23, wherein said biotin synthase comprises the amino acid sequence set forth in SEQ ID NO:21 or SEQ ID NO:24.

25. The method of embodiment 18, wherein said nucleotide sequence encodes an inhibitory nucleotide molecule that is capable of being transcribed as an inhibitory polynucleotide selected from the group consisting of a single-stranded RNA polynucleotide, a double-stranded RNA polynucleotide, and a combination thereof.

26. The method of embodiment 25, wherein said essential compound is an amino acid.

27. The method of embodiment 26, wherein said amino acid is isoleucine.

28. The method of embodiment 27, wherein said component of said biosynthetic pathway is threonine deaminase (TD), wherein said TD comprises an amino acid sequence having at least 90% sequence identity to the sequence set forth in SEQ ID NO:3 or SEQ ID NO:6.

29. The method of embodiment 28, wherein said TD comprises the amino acid sequence set forth in SEQ ID NO:3 or SEQ ID NO:6.

30. The method of embodiment 28 or embodiment 29, wherein said nucleotide sequence comprises a sequence selected from the group consisting of:
(a) the nucleotide sequence set forth in SEQ ID NO: 1 or 2, or a complement thereof;
(b) the nucleotide sequence set forth in SEQ ID NO:4 or 5, or a complement thereof,
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

31. The method of embodiment 28 or embodiment 29, wherein said nucleotide sequence comprises in the 5′-to-3′ orientation and operably linked:
(a) a TD forward fragment, said TD forward fragment comprising about 500 to about 800 contiguous nucleotides having at least 90% sequence identity to a nucleotide sequence of about 500 to about 800 contiguous nucleotides of SEQ ID NO:1, 2, 4, or 5;
(b) a spacer sequence comprising about 200 to about 700 nucleotides;
(c) and a TD reverse fragment, said TD reverse fragment having sufficient length and sufficient complementarity to said TD forward fragment such that said first nucleotide sequence is transcribed as an RNA molecule capable of forming a hairpin RNA structure.

32. The method of embodiment 31, wherein said TD reverse fragment comprises the complement of said TD forward fragment or a sequence having at least 90% sequence identity to the complement of said TD forward fragment.

33. The method of embodiment 31 or embodiment 32, wherein said spacer sequence comprises about 200 to about 700 nucleotides immediately downstream of said TD forward fragment.

34. The method of embodiment 31 or embodiment 32, wherein said spacer sequence comprises an intron.

35. The method of embodiment 26, wherein said amino acid is glutamine.

36. The method of embodiment 35, wherein said component of said biosynthetic pathway is selected from the group consisting of:
(a) glutamine synthase 1 (GS1), wherein said GS1 comprises an amino acid sequence having at least 90% sequence identity to the sequence set forth in SEQ ID NO:9 or SEQ ID NO:12;
(b) glutamine synthase 2 (GS2), wherein said GS2 comprises an amino acid sequence having at least 90% sequence identity to the sequence set forth in SEQ ID NO:15 or SEQ ID NO:18; and
(c) a combination of said GS1 and said GS2.

37. The method of embodiment 36, wherein said GS1 comprises the amino acid sequence set forth in SEQ ID NO:9 or SEQ ID NO:12.

38. The method of embodiment 36, wherein said GS2 comprises the amino acid sequence set forth in SEQ ID NO:15 or SEQ ID NO:18.

39. The method of embodiment 36 or embodiment 37, wherein said component is GS1, and wherein said nucleotide sequence comprises a sequence selected from the group consisting of:
(a) the nucleotide sequence set forth in SEQ ID NO:7 or 8, or a complement thereof;
(b) the nucleotide sequence set forth in SEQ ID NO:10 or 11, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

40. The method of embodiment 36 or embodiment 37, wherein said nucleotide sequence comprises in the 5′-to-3′ orientation and operably linked:
(a) a GS1 forward fragment, said GS1 forward fragment comprising about 500 to about 800 contiguous nucleotides having at least 90% sequence identity to a nucleotide sequence of about 500 to about 800 contiguous nucleotides of SEQ ID NO:7, 8, 10, or 11;
(b) a spacer sequence comprising about 200 to about 700 nucleotides;
(c) and a GS1 reverse fragment, said GS1 reverse fragment having sufficient length and sufficient complementarity to said GS1 forward fragment such that said first nucleotide sequence is transcribed as an RNA molecule capable of forming a hairpin RNA structure.

41. The method of embodiment 40, wherein said GS1 reverse fragment comprises the complement of said GS1 forward fragment or a sequence having at least 90% sequence identity to the complement of said GS1 forward fragment.

42. The method of embodiment 40 or embodiment 41, wherein said spacer sequence comprises about 200 to about 700 nucleotides immediately downstream of said GS1 forward fragment.

43. The method of embodiment 40 or embodiment 41, wherein said spacer sequence comprises an intron.

44. The method of embodiment 36 or embodiment 38, wherein said component is GS2, and wherein said nucleotide sequence comprises a sequence selected from the group consisting of:
(a) the nucleotide sequence set forth in SEQ ID NO:13 or 14, or a complement thereof;
(b) the nucleotide sequence set forth in SEQ ID NO:16 or 17, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

45. The method of embodiment 36 or embodiment 38, wherein said nucleotide sequence comprises in the 5′-to-3′ orientation and operably linked:
(a) a GS2 forward fragment, said GS2 forward fragment comprising about 500 to about 800 contiguous nucleotides having at least 90% sequence identity to a nucleotide sequence of about 500 to about 800 contiguous nucleotides of SEQ ID NO:13, 14, 16, or 17;
(b) a spacer sequence comprising about 200 to about 700 nucleotides;
(c) and a GS2 reverse fragment, said GS2 reverse fragment having sufficient length and sufficient complementarity to said GS2 forward fragment such that said first nucleotide sequence is transcribed as an RNA molecule capable of forming a hairpin RNA structure.

46. The method of embodiment 45, wherein said GS2 reverse fragment comprises the complement of said GS2 forward fragment or a sequence having at least 90% sequence identity to the complement of said GS2 forward fragment.

47. The method of embodiment 45 or embodiment 46, wherein said spacer sequence comprises about 200 to about 700 nucleotides immediately downstream of said GS2 forward fragment.

48. The method of embodiment 45 or embodiment 46, wherein said spacer sequence comprises an intron.

49. The method of any one of embodiments 36-38, wherein said component is a combination of said GS1 and said GS2, and wherein said nucleotide sequence comprises a fusion polynucleotide that is capable inhibiting expression of said GS1 and said GS2 in said duckweed plant or duckweed plant cell or nodule, wherein said fusion polynucleotide comprises in the 5′-to-3′ orientation and operably linked:
(a) a chimeric forward fragment, said chimeric forward fragment comprising in either order:
   (i) a first fragment comprising about 500 to about 650 contiguous nucleotides having at least 90% sequence identity to a nucleotide sequence of about 500 to about 650 contiguous nucleotides of a polynucleotide encoding said GS1; and
   (ii) a second fragment comprising about 500 to about 650 contiguous nucleotides having at least 90% sequence identity to a nucleotide sequence of about 500 to about 650 contiguous nucleotides of a polynucleotide encoding said GS2;
(b) a spacer sequence comprising about 200 to about 700 nucleotides; and
(c) a reverse fragment, said reverse fragment having sufficient length and sufficient complementarity to said chimeric forward fragment such that said fusion polynucleotide is transcribed as an RNA molecule capable of forming a hairpin RNA structure.

50. The method of embodiment 49, wherein said first fragment comprises about 500 to about 650 contiguous nucleotides having at least 90% sequence identity to a nucleotide sequence of about 500 to about 650 contiguous nucleotides of SEQ ID NO:7, 8, 10, or 11; and said second fragment comprises about 500 to about 650 contiguous nucleotides having at least 90% sequence identity to a nucleotide sequence of about 500 to about 650 contiguous nucleotides of SEQ ID NO: 13, 14, 16, or 17.

51. The method of embodiment 50, wherein said reverse fragment comprises the complement of said chimeric forward fragment or a sequence having at least 90% sequence identity to the complement of said chimeric forward fragment.

52. The method of any one of embodiments 49-51, wherein said spacer sequence comprises about 200 to about 700 nucleotides immediately downstream of said second fragment of said chimeric forward fragment.

53. The method of embodiment 52, wherein:
(a) said chimeric forward fragment comprises a first fragment of about 500 to about 650 contiguous nucleotides of SEQ ID NO:7, 8, 10, or 11 and a second fragment of about 500 to about 650 contiguous nucleotides of SEQ ID NO:13, 14, 16, or 17, and wherein said spacer sequence comprises about 200 to about 700 nucleotides immediately downstream of said second fragment; or
(b) said chimeric forward fragment comprises a first fragment of about 500 to about 650 contiguous nucleotides of SEQ ID NO:13, 14, 16, or 17 and a second fragment of about 500 to about 650 contiguous nucleotides of SEQ ID NO:7, 8, 10, or 11, and wherein said spacer sequence comprises about 200 to about 700 nucleotides immediately downstream of said second fragment.

54. The method of any one of embodiments 49-51, wherein said spacer sequence comprises an intron.

55. The method of embodiment 25, wherein said essential compound is a vitamin, and wherein said vitamin is biotin.

56. The method of embodiment 55, wherein said component of said biosynthetic pathway is biotin synthase (BS), wherein said BS comprises an amino acid sequence having at least 90% sequence identity to the sequence set forth in SEQ ID NO:21 or SEQ ID NO:24.

57. The method of embodiment 56, wherein said BS comprises the amino acid sequence set forth in SEQ ID NO:21 or SEQ ID NO:24.

58. The method of embodiment 56 or embodiment 57, wherein said nucleotide sequence comprises a sequence selected from the group consisting of:
(a) the nucleotide sequence set forth in SEQ ID NO: 19 or 20, or a complement thereof;
(b) the nucleotide sequence set forth in SEQ ID NO:22 or 23, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

59. The method of embodiment 56 or embodiment 57, wherein said nucleotide sequence comprises in the 5′-to-3′ orientation and operably linked:
(a) a BS forward fragment, said BS forward fragment comprising about 500 to about 800 contiguous nucleotides having at least 90% sequence identity to a nucleotide sequence of about 500 to about 800 contiguous nucleotides of SEQ ID NO:19, 20, 22, or 23;
(b) a spacer sequence comprising about 200 to about 700 nucleotides;
(c) and a BS reverse fragment, said BS reverse fragment having sufficient length and sufficient complementarity to said BS forward fragment such that said first nucleotide sequence is transcribed as an RNA molecule capable of forming a hairpin RNA structure.

60. The method of embodiment 59, wherein said BS reverse fragment comprises the complement of said BS forward fragment or a sequence having at least 90% sequence identity to the complement of said BS forward fragment.

61. The method of embodiment 59 or embodiment 60, wherein said spacer sequence comprises about 200 to about 700 nucleotides immediately downstream of said BS forward fragment.

62. The method of embodiment 59 or embodiment 60, wherein said spacer sequence comprises an intron.

63. The method of any one of embodiments 18-62, wherein said promoter is a constitutive promoter.

64. The method of embodiment 63, wherein said promoter is selected from the group consisting of the Superpromoter, the Spirodela polyrrhiza promoter, and a functional fragment thereof.

65. The method of any one of embodiments 15-64, wherein said heterologous polynucleotide of interest encodes a heterologous polypeptide of interest.

66. The method of embodiment 65, wherein said heterologous polypeptide of interest is a mammalian polypeptide or biologically active variant thereof.

67. The method of embodiment 66, wherein the polypeptide of interest is selected from the group consisting of insulin, growth hormone, α-interferon, β-interferon, β-glucocerebrosidase, β-glucoronidase, retinoblastoma protein, p53 protein, angiostatin, leptin, erythropoietin (EPO), granulocyte macrophage colony stimulating factor, plasminogen, tissue plasminogen activator, blood coagulation factors, alpha 1-antitrypsin, a monoclonal antibody (mAbs), a Fab fragment, a single-chain antibody, cytokines, receptors, hormones, human vaccines, animal vaccines, peptides, and serum albumin.

68. The method of any one of embodiments 15-64, wherein said heterologous polynucleotide of interest comprises a nucleotide sequence that inhibits expression or function of a target gene of interest, wherein said target gene of interest is other than a gene encoding for a component of a biosynthetic pathway for an essential compound.

69. The method of any one of embodiments 15-68, wherein said duckweed plant, or said duckweed plant cell or nodule, is from a genus selected from the group consisting of the genus Spirodela, genus Wolffla, genus Wolflella, genus Landoltia, and genus Lemna.

70. The method of embodiment 69, wherein said duckweed plant, or said duckweed plant cell or nodule, is a member of a species selected from the group consisting of Lemna minor, Lemna miniscula, Lemna aequinoctialis, and Lemna gibba.

71. The method of any one of embodiments 1-70, wherein said auxotrophic requirement is introduced into said plant, plant part, plant cell, or nodule prior to introducing said heterologous polynucleotide of interest into said plant, plant part, plant cell, or nodule.

72. The method of any one of embodiments 1-70, wherein said auxotrophic requirement is introduced into said plant, plant part, plant cell, or nodule after said heterologous polynucleotide of interest has been introduced into said plant, plant part, plant cell, or nodule.

73. The method of any one of embodiments 1-70, wherein said auxotrophic requirement and said heterologous polynucleotide of interest are introduced into said plant, plant part, plant cell, or nodule at the same time.

74. A method of making a duckweed plant, plant cell, or nodule having an auxotrophic requirement for an essential compound, said method comprising:
(a) expressing a polynucleotide or polypeptide in said duckweed plant, plant cell, or nodule, wherein said polynucleotide or polypeptide inhibits expression or function of a component of a biosynthetic pathway for said essential compound;
(b) eliminating a gene in said duckweed plant, plant cell, or nodule, wherein said gene encodes said component of said biosynthetic pathway for said essential compound; and
(c) mutating a gene in said duckweed plant, plant cell, or nodule, wherein said gene encodes said component of said biosynthetic pathway for said essential compound.

75. The method of embodiment 74, wherein said duckweed plant, plant cell, or nodule is stably transformed with a polynucleotide construct having a nucleotide sequence that is capable of inhibiting expression or function of said component of said biosynthetic pathway for said essential compound, said nucleotide sequence being operably linked to a promoter that is functional in a plant cell.

76. The method of embodiment 75, wherein said nucleotide sequence encodes a polypeptide that inhibits function of said component of said biosynthetic pathway.

77. The method of embodiment 76, wherein said polypeptide is an antibody or a binding protein that binds said component of the biosynthetic pathway for said essential compound, thereby inhibiting function of said component.

78. The method of embodiment 76 or embodiment 77, wherein said component of said biosynthetic pathway is biotin synthase.

79. The method of embodiment 78, wherein said nucleotide sequence encodes streptavidin or a fragment thereof that binds biotin synthase, thereby inhibiting function of said biotin synthase.

80. The method of embodiment 78 or embodiment 79, wherein said biotin synthase comprises an amino acid sequence having at least 90% sequence identity to the sequence set forth in SEQ ID NO:21 or SEQ ID NO:24.

81. The method of embodiment 80, wherein said biotin synthase comprises the amino acid sequence set forth in SEQ ID NO:21 or SEQ ID NO:24.

82. The method of embodiment 75, wherein said nucleotide sequence encodes an inhibitory nucleotide molecule that is capable of being transcribed as an inhibitory polynucleotide selected from the group consisting of a single-stranded RNA polynucleotide, a double-stranded RNA polynucleotide, and a combination thereof.

83. The method of embodiment 82, wherein said essential compound is an amino acid.

84. The method of embodiment 83, wherein said amino acid is isoleucine.

85. The method of embodiment 84, wherein said component of said biosynthetic pathway is threonine deaminase (TD), wherein said TD comprises an amino acid sequence having at least 90% sequence identity to the sequence set forth in SEQ ID NO:3 or SEQ ID NO:6.

86. The method of embodiment 85, wherein said TD comprises the amino acid sequence set forth in SEQ ID NO:3 or SEQ ID NO:6.

87. The method of embodiment 85 or embodiment 86, wherein said nucleotide sequence comprises a sequence selected from the group consisting of:
(a) the nucleotide sequence set forth in SEQ ID NO:1 or 2, or a complement thereof;
(b) the nucleotide sequence set forth in SEQ ID NO:4 or 5, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

88. The method of embodiment 85 or embodiment 86, wherein said nucleotide sequence comprises in the 5′-to-3′ orientation and operably linked:
(a) a TD forward fragment, said TD forward fragment comprising about 500 to about 800 contiguous nucleotides having at least 90% sequence identity to a nucleotide sequence of about 500 to about 800 contiguous nucleotides of SEQ ID NO:1, 2, 4, or 5;
(b) a spacer sequence comprising about 200 to about 700 nucleotides;
(c) and a TD reverse fragment, said TD reverse fragment having sufficient length and sufficient complementarity to said TD forward fragment such that said first nucleotide sequence is transcribed as an RNA molecule capable of forming a hairpin RNA structure.

89. The method of embodiment 88, wherein said TD reverse fragment comprises the complement of said TD forward fragment or a sequence having at least 90% sequence identity to the complement of said TD forward fragment.

90. The method of embodiment 88 or embodiment 89, wherein said spacer sequence comprises about 200 to about 700 nucleotides immediately downstream of said TD forward fragment.

91. The method of embodiment 88 or embodiment 89, wherein said spacer sequence comprises an intron.

92. The method of embodiment 83, wherein said amino acid is glutamine.

93. The method of embodiment 92, wherein said component of said biosynthetic pathway is selected from the group consisting of:
(a) glutamine synthase 1 (GS1), wherein said GS1 comprises an amino acid sequence having at least 90% sequence identity to the sequence set forth in SEQ ID NO:9 or SEQ ID NO:12;
(b) glutamine synthase 2 (GS2), wherein said GS2 comprises an amino acid sequence having at least 90% sequence identity to the sequence set forth in SEQ ID NO:15 or SEQ ID NO:18; and
(c) a combination of said GS1 and said GS2.

94. The method of embodiment 93, wherein said GS1 comprises the amino acid sequence set forth in SEQ ID NO:9 or SEQ ID NO:12.

95. The method of embodiment 93, wherein said GS2 comprises the amino acid sequence set forth in SEQ ID NO:15 or SEQ ID NO:18.

96. The method of embodiment 93 or embodiment 94, wherein said component is GS1, and wherein said nucleotide sequence comprises a sequence selected from the group consisting of:
(a) the nucleotide sequence set forth in SEQ ID NO:7 or 8, or a complement thereof;
(b) the nucleotide sequence set forth in SEQ ID NO:10 or 11, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

97. The method of embodiment 93 or embodiment 94, wherein said nucleotide sequence comprises in the 5′-to-3′ orientation and operably linked:
(a) a GS1 forward fragment, said GS1 forward fragment comprising about 500 to about 800 contiguous nucleotides having at least 90% sequence identity to a nucleotide sequence of about 500 to about 800 contiguous nucleotides of SEQ ID NO:7, 8, 10, or 11;
(b) a spacer sequence comprising about 200 to about 700 nucleotides;
(c) and a GS1 reverse fragment, said GS1 reverse fragment having sufficient length and sufficient complementarity to said GS1 forward fragment such that said first nucleotide sequence is transcribed as an RNA molecule capable of forming a hairpin RNA structure.

98. The method of embodiment 97, wherein said GS1 reverse fragment comprises the complement of said GS1 forward fragment or a sequence having at least 90% sequence identity to the complement of said GS1 forward fragment.

99. The method of embodiment 97 or embodiment 98, wherein said spacer sequence comprises about 200 to about 700 nucleotides immediately downstream of said GS1 forward fragment.

100. The method of embodiment 97 or embodiment 98, wherein said spacer sequence comprises an intron.

101. The method of embodiment 93 or embodiment 95, wherein said component is GS2, and wherein said nucleotide sequence comprises a sequence selected from the group consisting of:
(a) the nucleotide sequence set forth in SEQ ID NO:13 or 14, or a complement thereof;
(b) the nucleotide sequence set forth in SEQ ID NO:16 or 17, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

102. The method of embodiment 93 or embodiment 95, wherein said nucleotide sequence comprises in the 5′-to-3′ orientation and operably linked:
(a) a GS2 forward fragment, said GS2 forward fragment comprising about 500 to about 800 contiguous nucleotides having at least 90% sequence identity to a nucleotide sequence of about 500 to about 800 contiguous nucleotides of SEQ ID NO:13, 14, 16, or 17;
(b) a spacer sequence comprising about 200 to about 700 nucleotides;
(c) and a GS2 reverse fragment, said GS2 reverse fragment having sufficient length and sufficient complementarity to said GS2 forward fragment such that said first nucleotide sequence is transcribed as an RNA molecule capable of forming a hairpin RNA structure.

103. The method of embodiment 102, wherein said GS2 reverse fragment comprises the complement of said GS2 forward fragment or a sequence having at least 90% sequence identity to the complement of said GS2 forward fragment.

104. The method of embodiment 102 or embodiment 103, wherein said spacer sequence comprises about 200 to about 700 nucleotides immediately downstream of said GS2 forward fragment.

105. The method of embodiment 102 or embodiment 103, wherein said spacer sequence comprises an intron.

106. The method of any one of embodiments 93-95, wherein said component is a combination of said GS1 and said GS2, and wherein said nucleotide sequence comprises a fusion polynucleotide that is capable inhibiting expression of said GS1 and said GS2 in said duckweed plant or duckweed plant cell or nodule, wherein said fusion polynucleotide comprises in the 5′-to-3′ orientation and operably linked:
(a) a chimeric forward fragment, said chimeric forward fragment comprising in either order:
   (i) a first fragment comprising about 500 to about 650 contiguous nucleotides having at least 90% sequence identity to a nucleotide sequence of about 500 to about 650 contiguous nucleotides of a polynucleotide encoding said GS1; and
   (ii) a second fragment comprising about 500 to about 650 contiguous nucleotides having at least 90% sequence identity to a nucleotide sequence of about 500 to about 650 contiguous nucleotides of a polynucleotide encoding said GS2;
(b) a spacer sequence comprising about 200 to about 700 nucleotides; and
(c) a reverse fragment, said reverse fragment having sufficient length and sufficient complementarity to said chimeric forward fragment such that said fusion polynucleotide is transcribed as an RNA molecule capable of forming a hairpin RNA structure.

107. The method of embodiment 106, wherein said first fragment comprises about 500 to about 650 contiguous nucleotides having at least 90% sequence identity to a nucleotide sequence of about 500 to about 650 contiguous nucleotides of SEQ ID NO:7, 8, 10, or 11; and said second fragment comprises about 500 to about 650 contiguous nucleotides having at least 90% sequence identity to a nucleotide sequence of about 500 to about 650 contiguous nucleotides of SEQ ID NO:13, 14, 16, or 17.

108. The method of embodiment 107, wherein said reverse fragment comprises the complement of said chimeric forward fragment or a sequence having at least 90% sequence identity to the complement of said chimeric forward fragment.

109. The method of any one of embodiments 106-108, wherein said spacer sequence comprises about 200 to about 700 nucleotides immediately downstream of said second fragment of said chimeric forward fragment.

110. The method of embodiment 109, wherein:
(a) said chimeric forward fragment comprises a first fragment of about 500 to about 650 contiguous nucleotides of SEQ ID NO:7, 8, 10, or 11 and a second fragment of about 500 to about 650 contiguous nucleotides of SEQ ID NO:13, 14, 16, or 17, and wherein said spacer sequence comprises about 200 to about 700 nucleotides immediately downstream of said second fragment; or
(b) said chimeric forward fragment comprises a first fragment of about 500 to about 650 contiguous nucleotides of SEQ ID NO:13, 14, 16, or 17 and a second fragment of about 500 to about 650 contiguous nucleotides of SEQ ID NO:7, 8, 10, or 11, and wherein said spacer sequence comprises about 200 to about 700 nucleotides immediately downstream of said second fragment.

111. The method of any one of embodiments 106-108, wherein said spacer sequence comprises an intron.

112. The method of embodiment 82, wherein said essential compound is a vitamin, and wherein said vitamin is biotin.

113. The method of embodiment 112, wherein said component of said biosynthetic pathway is biotin synthase (BS), wherein said BS comprises an amino acid sequence having at least 90% sequence identity to the sequence set forth in SEQ ID NO:21 or SEQ ID NO:24.

114. The method of embodiment 113, wherein said BS comprises the amino acid sequence set forth in SEQ ID NO:21 or SEQ ID NO:24.

115. The method of embodiment 113 or embodiment 114, wherein said nucleotide sequence comprises a sequence selected from the group consisting of:
(a) the nucleotide sequence set forth in SEQ ID NO:19 or 20, or a complement thereof;
(b) the nucleotide sequence set forth in SEQ ID NO:22 or 23, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

116. The method of embodiment 113 or embodiment 114, wherein said nucleotide sequence comprises in the 5′-to-3′ orientation and operably linked:
(a) a BS forward fragment, said BS forward fragment comprising about 500 to about 800 contiguous nucleotides having at least 90% sequence identity to a nucleotide sequence of about 500 to about 800 contiguous nucleotides of SEQ ID NO:19, 20, 22, or 23;
(b) a spacer sequence comprising about 200 to about 700 nucleotides;
(c) and a BS reverse fragment, said BS reverse fragment having sufficient length and sufficient complementarity to said BS forward fragment such that said first nucleotide sequence is transcribed as an RNA molecule capable of forming a hairpin RNA structure.

117. The method of embodiment 116, wherein said BS reverse fragment comprises the complement of said BS forward fragment or a sequence having at least 90% sequence identity to the complement of said BS forward fragment.

118. The method of embodiment 116 or embodiment 117, wherein said spacer sequence comprises about 200 to about 700 nucleotides immediately downstream of said BS forward fragment.

119. The method of embodiment 116 or embodiment 117, wherein said spacer sequence comprises an intron.

120. The method of any one of embodiments 75-119, wherein said promoter is a constitutive promoter.

121. The method of embodiment 120, wherein said promoter is selected from the group consisting of the Superpromoter, the Spirodela polyrrhiza promoter, and a functional fragment thereof.

122. The method of any one of embodiments 74-121, wherein said duckweed plant, plant cell, or nodule comprises a heterologous polynucleotide of interest encoding a heterologous polypeptide of interest.

123. The method of embodiment 122, wherein said heterologous polypeptide of interest is a mammalian polypeptide or biologically active variant thereof.

124. The method of embodiment 123, wherein the polypeptide of interest is selected from the group consisting of insulin, growth hormone, α-interferon, β-interferon, β-glucocerebrosidase, β-glucoronidase, retinoblastoma protein, p53 protein, angiostatin, leptin, erythropoietin (EPO), granulocyte macrophage colony stimulating factor, plasminogen, tissue plasminogen activator, blood coagulation factors, alpha 1-antitrypsin, a monoclonal antibody (mAbs), a Fab fragment, a single-chain antibody, cytokines, receptors, hormones, human vaccines, animal vaccines, peptides, and serum albumin.

125. The method of any one of embodiments 74-121, wherein said duckweed plant, plant cell, or nodule comprises a heterologous polynucleotide of interest comprising a nucleotide sequence that inhibits expression or function of a target gene of interest, wherein said target gene of interest is other than a gene encoding for a component of a biosynthetic pathway for an essential compound.

126. The method of any one of embodiments 74-125, wherein said duckweed plant, or said duckweed plant cell or nodule, is from a genus selected from the group consisting of the genus Spirodela, genus Wolffia, genus Wolfliella, genus Landoltia, and genus Lemna.

127. The method of embodiment 126, wherein said duckweed plant, or said duckweed plant cell or nodule, is a member of a species selected from the group consisting of Lemna minor, Lemna miniscula, Lemna aequinoctialis, and Lemna gibba.

128. The method of any one of embodiments 122-127, wherein said auxotrophic requirement is introduced into said duckweed plant, plant cell, or nodule prior to introducing said heterologous polynucleotide of interest into said duckweed plant, plant cell, or nodule.

129. The method of any one of embodiments 122-127, wherein said auxotrophic requirement is introduced into said duckweed plant, plant cell, or nodule after said heterologous polynucleotide of interest has been introduced into said duckweed plant, plant cell, or nodule.

130. The method of any one of embodiments 122-127, wherein said auxotrophic requirement and said heterologous polynucleotide of interest are introduced into said duckweed plant, plant cell, or nodule at the same time.

131. A method of making a transgenic plant or plant part having an auxotrophic requirement, wherein said transgenic plant or plant part comprises a heterologous polynucleotide of interest, said method comprising introducing into said transgenic plant or plant part a polynucleotide construct having a nucleotide sequence that is capable of inhibiting expression or function of a component of a biosynthetic pathway for an essential compound, said nucleotide sequence being operably linked to a promoter that is functional in a plant cell.

132. The method of embodiment 131, wherein said essential compound is an amino acid, a carbohydrate, a fatty acid, a nucleic acid, a vitamin, a plant hormone, or a precursor thereof.

133. The method of embodiment 131 or embodiment 132, wherein said nucleotide sequence encodes an inhibitory nucleotide molecule that is capable of being transcribed as an inhibitory polynucleotide selected from the group consisting of a single-stranded RNA polynucleotide, a double-stranded RNA polynucleotide, and a combination thereof.

134. The method of embodiment 131 or embodiment 132, wherein said nucleotide sequence encodes a polypeptide that inhibits function of said component of said biosynthetic pathway.

135. The method of embodiment 134, wherein said polypeptide is an antibody or a binding protein that binds said component of the biosynthetic pathway for said essential compound, thereby inhibiting function of said component.

136. The method of any one of embodiments 131-135, wherein said promoter is a constitutive promoter.

137. The method of any one of embodiments 131-136, wherein said heterologous polynucleotide of interest encodes a heterologous polypeptide of interest, or wherein said heterologous polynucleotide of interest comprises a nucleotide sequence that inhibits expression or function of a target gene of interest, wherein said target gene of interest is other than a gene encoding for a component of a biosynthetic pathway for an essential compound.

138. The method of embodiment 137, wherein said heterologous polypeptide of interest is a mammalian polypeptide or biologically active variant thereof.

139. The method of embodiment 138, wherein the polypeptide of interest is selected from the group consisting of insulin, growth hormone, α-interferon, β-interferon, β-glucocerebrosidase, β-glucoronidase, retinoblastoma protein, p53 protein, angiostatin, leptin, erythropoietin (EPO), granulocyte macrophage colony stimulating factor, plasminogen, tissue plasminogen activator, blood coagulation factors, alpha 1-antitrypsin, a monoclonal antibody (mAbs), a Fab fragment, a single-chain antibody, cytokines, receptors, hormones, human vaccines, animal vaccines, peptides, and serum albumin.

140. The method of any one of embodiments 131-139, wherein said plant is a monocot.

141. The method of any one of embodiments 131-139, wherein said plant is a dicot.

142. The method of any one of embodiments 131-141, wherein said auxotrophic requirement is introduced into said plant or plant part prior to introducing said heterologous polynucleotide of interest into said plant or plant part.

143. The method of any one of embodiments 131-141, wherein said auxotrophic requirement is introduced into said plant or plant part after said heterologous polynucleotide of interest has been introduced into said plant or plant part.

144. The method of any one of embodiments 131-141, wherein said auxotrophic requirement and said heterologous polynucleotide of interest are introduced into said plant or plant part at the same time.

145. A plant, plant part, plant cell, or nodule according to any one of embodiments 74-144.

146. A method of regulating production of a heterologous polypeptide of interest in a transgenic plant or plant part having at least one auxotrophic requirement for an essential compound, wherein said transgenic plant or plant part comprises a heterologous polynucleotide encoding said polypeptide of interest operably linked to a promoter that is functional in a plant cell, said method comprising:
providing an effective amount of said essential compound to said transgenic plant or plant part under culture conditions suitable for expression and production of said heterologous polypeptide, wherein said transgenic plant or plant part grows in the presence of said effective amount of said essential compound and said heterologous polypeptide is produced; and
removing said essential compound from said transgenic plant or plant part, wherein growth of said transgenic plant or plant part is inhibited in the absence of said compound, whereby expression and production of said heterologous polypeptide is reduced.

147. The method of embodiment 146, wherein said transgenic plant or plant part is a transgenic plant or plant part according to any one of embodiments 137-144.

148. The method of embodiment 146, wherein said transgenic plant or plant part is a duckweed plant, plant cell, or nodule according to any one of embodiments 122-124 and 126-130.

149. An isolated polynucleotide comprising a nucleotide sequence selected from the group consisting of:
(a) the nucleotide sequence set forth in SEQ ID NO:1, 2, 4, or 5;
(b) the nucleotide sequence set forth in SEQ ID NO:7, 8, 10, or 11;
(c) the nucleotide sequence set forth in SEQ ID NO:13, 14, 16, or 17;
(d) the nucleotide sequence set forth in SEQ ID NO:19, 20, 22, or 23;
(e) a nucleotide sequence encoding a polypeptide comprising the amino acid sequence set forth in SEQ ID NO:3, 6, 9, 12, 15, 18, 21, or 24;
(f) a nucleotide sequence comprising at least 90% sequence identity to the sequence set forth in SEQ ID NO:1, 2, 4, or 5, wherein said polynucleotide encodes a polypeptide having threonine deaminase (TD) activity;
(g) a nucleotide sequence comprising at least 90% sequence identity to the sequence set forth in SEQ ID NO:7, 8, 10, or 11, wherein said polynucleotide encodes a polypeptide having glutamine synthetase 1 (GS1) activity;
(h) a nucleotide sequence comprising at least 90% sequence identity to the sequence set forth in SEQ ID NO:13, 14, 16, or 17, wherein said polynucleotide encodes a polypeptide having glutamine synthetase 2 (GS2) activity;
(i) a nucleotide sequence comprising at least 90% sequence identity to the sequence set forth in SEQ ID NO:19, 20, 22, or 23, wherein said polynucleotide encodes a polypeptide having biotin synthase (BS) activity;
(j) a nucleotide sequence comprising at least 15 contiguous nucleotides of SEQ ID NO:1, 2, 4, 5, 7, 8, 10, 11, 13, 14, 16, 17, 19, 20, 22, or 23, or a complement thereof;
(k) a nucleotide sequence comprising at least 19 contiguous nucleotides having at least 90% sequence identity to a nucleotide sequence comprising at least 19 contiguous nucleotides of SEQ ID NO:1, 2, 4, 5, 7, 8, 10, 11, 13, 14, 16, 17, 19, 20, 22, or 23, and a complement thereof;
(l) a nucleotide sequence encoding an amino acid sequence having at least 90% sequence identity to the sequence set forth in SEQ ID NO:3 or 6, wherein said polynucleotide encodes a polypeptide having TD activity;
(m) a nucleotide sequence encoding an amino acid sequence having at least 90% sequence identity to the sequence set forth in SEQ ID NO:9 or 12, wherein said polynucleotide encodes a polypeptide having GS1 activity;
(n) a nucleotide sequence encoding an amino acid sequence having at least 90% sequence identity to the sequence set forth in SEQ ID NO:15 or 18, wherein said polynucleotide encodes a polypeptide having GS2 activity;
(o) a nucleotide sequence encoding an amino acid sequence having at least 90% sequence identity to the sequence set forth in SEQ ID NO:21 or 24, wherein said polynucleotide encodes a polypeptide having BS activity;
(p) the complement of the nucleotide sequence of any one of preceding items (a) through (O).

150. An expression construct or auxotrophic construct comprising the polynucleotide of embodiment 149 operably linked to a promoter that is functional in a plant cell.

151. A plant or plant cell comprising the expression construct or auxotrophic construct of embodiment 150.

152. The plant or plant cell of embodiment 151, wherein said plant is a monocot or said plant cell is from a monocot.

153. The plant or plant cell of embodiment 152, wherein said monocot is a member of the Lemnaceae.

154. The plant or plant cell of embodiment 153, wherein said monocot is from a genus selected from the group consisting of the genus Spirodela, genus Wolffla, genus Wolfiella, genus Landoltia, and genus Lemna.

155. The plant or plant cell of embodiment 154, wherein said monocot is a member of a species selected from the group consisting of Lemna minor, Lemna miniscula, Lemna aequinoctialis, and Lemna gibba.

156. The plant or plant cell of embodiment 151, wherein said plant is a dicot or said plant cell is from a dicot.

157. The plant or plant cell of any one of embodiments 151 through 156, wherein said polynucleotide is stably incorporated into the genome of the plant or plant cell.

158. An isolated polypeptide comprising an amino acid sequence selected from the group consisting of:
(a) the amino acid sequence set forth in SEQ ID NO:3 or 6;
(b) an amino acid sequence having at least 90% sequence identity to the amino acid sequence set forth in SEQ ID NO:3 or 6, wherein said polypeptide has threonine deaminase (TD) activity;
(c) an amino acid sequence comprising at least 20 consecutive amino acids of SEQ ID NO:3 or 6, wherein said polypeptide has TD activity;
(d) the amino acid sequence set forth in SEQ ID NO:9 or SEQ ID NO:12;
(e) an amino acid sequence having at least 90% sequence identity to the amino acid sequence set forth in SEQ ID NO:9 or SEQ ID NO:12, wherein said polypeptide has glutamine synthetase 1 (GS1) activity;
(f) an amino acid sequence comprising at least 20 consecutive amino acids of SEQ ID NO:9 or SEQ ID NO:12, wherein said polypeptide has GS1 activity;
(g) the amino acid sequence set forth in SEQ ID NO:15 or SEQ ID NO:18;
(h) an amino acid sequence having at least 90% sequence identity to the amino acid sequence set forth in SEQ ID NO:15 or SEQ ID NO:18, wherein said polypeptide has glutamine synthetase 2 (GS2) activity;
(i) an amino acid sequence comprising at least 20 consecutive amino acids of SEQ ID NO: 15 or SEQ ID NO: 18, wherein said polypeptide has GS2 activity;
(j) the amino acid sequence set forth in SEQ ID NO:21 or SEQ ID NO:24;
(k) an amino acid sequence having at least 90% sequence identity to the amino acid sequence set forth in SEQ ID NO:21 or SEQ ID NO:24, wherein said polypeptide has biotin synthase (BS) activity; and
(l) an amino acid sequence comprising at least 20 consecutive amino acids of SEQ ID NO:21 or SEQ ID NO:24, wherein said polypeptide has BS activity.

### BRIEF DESCRIPTION OF THE DRAWINGS

FIG. 1A sets forth the cDNA (SEQ ID NO:1; coding sequence set forth in SEQ ID NO:2; encoded protein set forth in SEQ ID NO:3) sequence for the Lemna minor threonine deaminase (TD) isoform #1. FIG. 1B sets forth the cDNA (SEQ ID NO:4; coding sequence set forth in SEQ ID NO:5; encoded protein set forth in SEQ ID NO:6) sequence for the L. minor TD isoform #2.

FIG. 2A sets forth the cDNA (SEQ ID NO:7; coding sequence set forth in SEQ ID NO:8; encoded protein set forth in SEQ ID NO:9) sequence for the Lemna minor glutamine synthetase 1 (GS1) isoform #1. FIG. 2B sets forth the cDNA (SEQ ID NO:10; coding sequence set forth in SEQ ID NO:11; encoded protein set forth in SEQ ID NO:12) sequence for the L. minor GS1 isoform #2.

FIG. 3A sets forth the cDNA (SEQ ID NO:13; coding sequence set forth in SEQ ID NO:14; encoded protein set forth in SEQ ID NO:15) sequence for the Lemna minor glutamine synthetase 2 (GS1) isoform #1. FIG. 3B sets forth the cDNA (SEQ ID NO:16; coding sequence set forth in SEQ ID NO:17; encoded protein set forth in SEQ ID NO: 18) sequence for the L. minor GS2 isoform #2.

FIG. 4A sets forth the cDNA (SEQ ID NO:19; coding sequence set forth in SEQ ID NO:20; encoded protein set forth in SEQ ID NO:21) sequence for the Lemna minor biotin synthase (BS) isoform #1. FIG. 4B sets forth the cDNA (SEQ ID NO:22; coding sequence set forth in SEQ ID NO:23; encoded protein set forth in SEQ ID NO:24) sequence for the L. minor BS isoform #2.

FIG. 5 sets forth one strategy for designing a single-gene RNAi knockout of Lemna minor threonine deaminase (TD), based on TD isoform #1.

FIG. 6 sets forth one strategy for designing a double-gene RNAi knockout of Lemna minor cytosol-localized glutamine synthetase 1 (GS1) and plastid-localized glutamine synthetase 2 (GS2), where the GS1 and GS2 portions of the RNAi knockout are based on the DNA sequences for GS1 isoform #1 and GS2 isoform #1, respectively.

FIG. 7 shows the AUXC01 vector with an auxotrophic construct comprising an RNAi expression cassette designed for single-gene RNAi knockout of Lemna minor threonine deaminase (TD). Expression of the TD inhibitory sequence (denoted by TD forward and TD reverse arrows; see FIG. 5) is driven by the operably linked Superpromoter (denoted as AocsAocsAocsAmasPmas) comprising three upstream activating sequences (Aocs) derived from the Agrobacterium tumefaciens octopine synthase gene operably linked to a promoter derived from an Agrobacterium tumefaciens mannopine synthase gene (AmasPmas). RbcS leader, rubisco small subunit leader sequence; ADH1, intron of maize alcohol dehydrogenase 1 gene; Tnos, Agrobacterium tumefacians nopaline synthase (nos) terminator sequence.

FIG. 8 shows the AUXC02 vector with an auxotrophic construct comprising an RNAi expression cassette designed for single-gene RNAi knockout of Lemna minor TD. For this construct, expression of the TD inhibitory sequence (again denoted by TD forward and TD reverse arros; see FIG. 5) is driven by the operably linked full-length Spirodela polyrrhiza ubiquitin promoter (designated SpUbq; see SEQ ID NO:40 of the present application).

FIG. 9 provides diagrams showing the general structure of Lemna minor threonine deaminase cDNA (TD) and the T-DNA regions of all binary transformation vectors. Abbreviations: 5′ and 3′, 5′ and 3′ UTR regions; HA, H5N1 avian influenza hemagglutitin gene; LB and RB, T-DNA left and right borders; M1, geneticin resistance marker gene; M2, kanamycin resistance marker gene; P1, Superpromoter, P2, SpUbq promoter (SEQ ID NO:40); P3, Truncated SpUbq promoter (SpUbq117; SEQ ID NO:41); qPCR, amplification region for quantitative real-time PCR; T1, nopaline synthase transcription terminator, TD, threonine deaminase gene.

FIG. 10 illustrates the optimal isoleucine concentration for growing selected auxotrophs. Fresh weights were taken from plants grown for 14 days in SH medium supplemented with 0, 0.25, 0.375, 0.5, and 1.0 mM isoleucine. All fresh weights were calculated relative to the wild-type Lemna grown without isoleucine supplement (set at 100%). Each bar and error bar represent the average and the standard deviation of triplicate samples, respectively.

FIG. 11A shows the level of endogenous threonine deaminase RNA in auxotrophic lines determined by real-time qPCR. For comparison, wild-type Lemna minor was grown with (+) and without (−) isoleucine supplement. The real-time PCR data was calculated relative to the level of wild type that was grown without any isoleucine (set at 100%). Each bar represents the average of two real-time PCR experiments, and the error bars represents the standard deviation. FIG. 11B shows relative biomass accumulation of different auxotrophic lines under optimal growth conditions. Fresh weights were taken from plants grown for 14 days in SH medium in the absence (solid box) and presence of isoleucine (hatched box, 0.25 mM). All fresh weights were calculated relative to the wild-type Lemna that was grown without isoleucine supplement (set at 100%). Each bar represents the average (values are displayed on top of each bar) of three independent experiments (run in triplicate) spanning over a 10-month period. Error bars represent the standard deviations of triplicates.

FIG. 12 shows the AUXD01 vector with an auxotrophic construct comprising a chimeric RNAi expression cassette designed for double-gene RNAi knockout of Lemna minor glutamine synthetase 1 (GS1) and glutamine synthetase 2 (GS2). The hairpin RNA is expressed as a chimeric sequence (a chimeric hairpin RNA), where fragments of the two genes are fused together and expressed as one transcript. Expression of the GS1/GS2 inhibitory sequence (denoted by GS1 and GS2 forward arrows and GS2 and GS1 reverse arrows; see FIG. 6) is driven by the operably linked Superpromoter (AocsAocsAocsAmasPmas expression control element). RbcS leader, rubisco small subunit leader sequence; ADH1, intron of maize alcohol dehydrogenase 1 gene; nos-ter, Agrobacterium tumefacians nopaline synthase (nos) terminator sequence.

FIG. 13 shows the AUXD02 vector with an auxotrophic construct comprising a chimerici RNAi expression cassette designed for double-gene RNAi knockout of Lemna minor GS1 and GS2. For this construct, expression of the GS1/GS2 inhibitory sequence (again denoted by the GS1 and GS2 forward arrows and GS2 and GS1 reverse arrows; see FIG. 6) is driven by the operably linked full-length Spirodela polyrrhiza ubiquitin promoter (designated SpUbq; see SEQ ID NO:40).

FIG. 14 shows the effect of glutamine concentration on fresh weight and dry weight of wild-type Lemna minor over a 14-day culture period.

FIG. 15 shows biomass accumulation for glutamine Lemna minor auxotrophic plant lines after 14 days growth in media lacking glutamine and media supplemented with 30 mM glutamine compared to wild-type plants.

FIG. 16 shows that the AUXD01 vector with the auxotrophic construct comprising the GS1/GS2 chimeric RNAi expression cassette effectively knocked down endogenous transcript levels of GS1 and GS2 in the glutamine Lemna minor auxotroph transformants. GS1 and GS2 mRNA transcript levels were analyzed by qPCR in several of these auxotrophic lines.

FIG. 17 shows the AUXA01 vector for streptavidin overexpression. Expression of the streptavidin protein is driven by the operably linked Superpromoter (AocsAocsAocsAmasPmas expression control element). RbcS leader, rubisco small subunit leader sequence; ADH1, intron of maize alcohol dehydrogenase 1 gene; nos-ter, Agrobacterium tumefacians nopaline synthase (nos) terminator sequence.

FIG. 18 shows the AUXA02 vector for overexpression of the core region of the streptavidin protein. Expression of the core region of the streptavidin protein is driven by the operably linked Superpromoter (AocsAocsAocsAmasPmas expression control element). RbcS leader, rubisco small subunit leader sequence; ADH1, intron of maize alcohol dehydrogenase 1 gene; nos-ter, Agrobacterium tumefacians nopaline synthase (nos) terminator sequence.

FIG. 19 shows the AUXB01 vector with an auxotrophic construct comprising an RNAi expression cassette designed for single-gene RNAi knockout of Lemna minor biotin sythase (BS). Expression of the BS inhibitory sequence (denoted by BS forward and BS reverse arrows) is driven by the operably linked Superpromoter (denoted as AocsAocsAocsAmasPmas. RbcS leader, rubisco small subunit leader sequence; ADH1, intron of maize alcohol dehydrogenase 1 gene; Tnos, Agrobacterium tumefacians nopaline synthase (nos) terminator sequence.

FIG. 20 shows the AUXB02 vector with an auxotrophic construct comprising an RNAi expression cassette designed for single-gene RNAi knockout of Lemna minor BS. For this construct, expression of the BS inhibitory sequence (again denoted by BS forward and BS reverse arrows) is driven by the operably linked full-length Spirodela polyrrhiza ubiquitin promoter (designated SpUbq; see SEQ ID NO:40).

FIG. 21 shows the effect of biotin concentration on fresh weight and dry weight of wild-type Lemna minor over a 7-day culture period.

### DETAILED DESCRIPTION

The present invention relates to the use of auxotrophy to biocontain transgenic plant material, thereby minimizing escape of heterologous genetic material from the transgenic plant or plant part into the environment and/or wild-type plant population. In this manner, the invention provides methods and compositions for introducing an auxotrophic requirement into a transgenic plant or plant part, as well as methods for biocontaining transgenic plants or plant parts based on this auxotrophic requirement. The auxotrophic requirement can be introduced using genetic engineering or mutagenesis that targets expression or function of a component of a biosynthetic pathway for an essential compound required for growth and/or survival of the transgenic plant or plant part. By “component” is intended any enzyme or coenzyme that participates in a biosynthetic pathway for the essential compound for which an auxotrophic requirement is to be introduced. Transgenic plants or plant parts having the auxotrophic requirement for the essential compound advantageously can be biocontained by providing the essential compound to allow for growth, followed by removal of the essential compound to inhibit or prevent further growth of the transgenic plant or plant part. In some embodiments, the invention provides novel polynucleotides and polynucleotide constructs for inhibiting expression or function of a component of a biosynthetic pathway for an essential compound. These polynucleotides and polynucleotide constructs can be utilized in the methods of the invention for introducing an auxotrophic requirement and biocontaining transgenic plants and plant parts.

While not intending to be bound to any particular theory or mechanism of action, transgenic plants and plant parts having at least one auxotrophic requirement for an essential compound, such as an amino acid, fatty acid, carbohydrate, nucleic acid, vitamin, plant hormone, or precursor thereof, will fail to develop, grow, or survive in its absence, thereby attenuating a risk of transfer of heterologous genetic material, for example, transgenes of interest, to the environment and conventional crops or wild-type plant populations. As such, compositions and methods are described herein for making and using transgenic plants and plant parts having at least one auxotrophic requirement for an essential compound. Transgenic plants and plants parts having at least one auxotrophic requirement are biocontained by withdrawal of the essential compound.

As used herein, “auxotroph,” “auxotrophy,” and “auxotrophic” means a plant or plant part thereof in which the plant or plant part is unable to synthesize a compound essential for its development, growth, or survival (hereinafter referred to as “an essential compound”), or if able to synthesize the essential compound is unable to utilize the compound efficiently, thus requiring uptake of the essential compound from its environment. Essential compounds are typically organic compounds and include, but are not limited to, amino acids, carbohydrates, fatty acids, nucleic acids, vitamins, plant hormones, and precursors thereof. The auxotrophic plant or plant part can be generated by introducing into the plant or plant part a mutation or inhibitory polynucleotide construct that targets expression or function of a component of a biosynthetic pathway for an essential compound, thereby rendering the plant or plant part unable to synthesize or utilize the essential compound. Auxotrophs therefore require supplementation with the essential compound, for example, an amino acid, carbohydrate, fatty acid, nucleic acid, vitamin, plant hormone, or precursor thereof, for development, growth, and/or survival.

As used herein, “auxotrophic requirement” means a need for exogenous supplementation of an essential compound such as an amino acid, carbohydrate, fatty acid, nucleic acid, vitamin, plant hormone, or precursor thereof for development, growth, and/or survival of a transgenic plant or plant part. By “exogenous supplementation” is intended the essential compound must be provided to the transgenic plant or plant part from a source that is external to the plant or plant part. Exogenous supplementation may be achieved by any application method known to those of skill in the art, including, but not limited to, foliar/stem application, application to the roots and/or the root environment, supplementation within a culture or plant growth medium, and the like.

As used herein, “biological containment,” “biocontainment,” and the like (e.g., “biocontain” and “biocontaining”) in the context of a transgenic plant or plant part means preventing the escape of transgenic plant material from a controlled environment into an uncontrolled environment. By “controlled environment” is intended the immediate environment in which the transgenic plant or plant part is being cultivated. Examples of controlled environments include, but are not limited to, laboratory settings, plant growth chambers, bioreactors, control field plots, and the like. By “uncontrolled environment” is intended any environment external to the “controlled” environment in which the transgenic plant or plant part is being grown or cultivated. By preventing escape of such transgenic material, the transfer of heterologous genetic material from the transgenic plant or plant part to conventional crops or wild-type plant populations can be minimized.

The present invention therefore broadly relates to methods and compositions for making and using an auxotrophic requirement for an essential compound such as an amino acid, carbohydrate, fatty acid, nucleic acid, vitamin, plant hormone, or precursor thereof, or any combination thereof, in transgenic plants and plant parts.

As used herein, “transgenic plant” and “transgenic plant part” means a plant or plant part that comprises a heterologous polynucleotide sequence of interest that is in addition to any heterologous nucleotide sequence that causes the auxotrophic requirement. By “heterologous” in the context of a polynucleotide sequence is intended that it originates from a foreign species, or, if from the same species, is substantially modified from its native form in composition and/or genomic locus by deliberate human intervention. Transgenic plants or transgenic plant parts include plants or plant parts that comprise polynucleotides encoding a heterologous polypeptide of interest (i.e., a polypeptide that is foreign to the plant host cell), as well as plants and plant parts that comprise inhibitory polynucleotides that target expression or function of a gene/protein of interest, where that gene/protein of interest is other than the gene(s)/protein(s) whose inhibition of expression and/or function results in the auxotrophic requirement. Regardless, it is to be noted that by transgenic is meant that the plant or plant part comprises heterologous genetic material other than or in addition to the heterologous genetic material that causes the auxotrophic requirement.

As used herein, “transgene” or “transgenes” means a polynucleotide encoding a foreign or heterologous polypeptide of interest, which is partly or entirely heterologous to the transgenic plant or plant part into which is introduced. A transgene contains optionally one or more transcriptional regulatory sequences and any other nucleic acid sequences, such as introns, that may be necessary for optimal expression of the transgene, all operably linked to the selected nucleic acid sequence. The transgene can be introduced into the plant or plant part by any method available in the art. Unless defined otherwise, all technical and scientific terms used herein have the same meaning as commonly understood by one of ordinary skill in the art to which the invention pertains. Many modifications and other embodiments of the inventions set forth herein will come to mind to one of ordinary skill in the art having the benefit of the teachings presented in the foregoing description and the associated drawings. Therefore, it is to be understood that the inventions are not to be limited to the specific embodiments disclosed and that modifications and other embodiments are intended to be included within the scope of the embodiments described herein and the appended claims. Although specific terms are employed herein, they are used in a generic and descriptive sense only and not for purposes of limitation.

### Overview

In one aspect, the present invention relates to compositions and methods for introducing and using an auxotrophic requirement for an amino acid in transgenic plants and plants parts. An amino acid has amino and carboxylate groups attached to an α-carbon, with each amino acid distinguished from the others by a different side chain (R group) attached to the α-carbon. Amino acids have fundamental roles both as building blocks of proteins and as intermediates in cellular metabolism. The ability of plants to synthesize the entire group of 20 amino acids is critical to their survival; therefore manipulation of a biosynthetic pathway for any one or more of these amino acids can serve as a means for introducing an auxotrophic requirement into a transgenic plant or plant part. Examples of amino acids suitable for introducing an auxotrophic requirement into a transgenic plant or plant part include, but are not limited to, any of the 20 amino acids, i.e., alanine, arginine, asparagine, aspartate, cysteine, glutamate, glutamine, glycine, histadine, isoleucine, leucine, lysine, methionine, phenylalanine, proline, serine, threonine, tryptophan, valine, and tyrosine. Any component within a biosynthetic pathway for one or more of these amino acids can be targeted at the gene or protein level to inhibit synthesis of the respective amino acid. For examples of plant genes and encoded proteins involved in biosynthesis of essential amino acids in plants see, for example, Betti et al. (2006) Planta 224:1068-1079; Colau et al. (1987) Mol. Cell. Biol. 7:2552-2557; El Malki and Jacobs (2001) Plant Mol. Biol. 45:191-199; Fankhauser et al. (1990) Planta 180:297-302; Hesse et al. (2004) J. Exp. Bot. 55:1799-1808; Kang et al. (2006) Plant Cell 18:3303-3320; Last and Fink (1988) Science 240:305-310; Logusch et al. (1991) Plant Physiol. 95:1057-1062; Martin et al. (2006) Plant Cell 18:3252-3274; Muralla et al. (2007) Plant Physiol. 144:890-903; Negrutiu et al. (1985) Mol. Gen. Genet. 199:330-337; Orea et al. (2002) Physiol. Plant. 115:352-361; Saito et al. (1992) Proc. Natl. Acad Sdt. USA 89:8078-8082; Stepanski and Leustek (2006) Amino Acids 30:127-142; Szamosi et al. (1994) Plant Physiol. 106(4):1257-1260; Tabuchi et al. (2005) Plant J. 42:641-651; Temple et al. (1993) Mol. Gen. Genet. 236:315-325; Wallsgrove et al. (1987) Plant Physiol. 83:155-158; U.S. Pat. Nos. 5,098,838, 5,145,777, 5,344,923, 5,747,308, 6,329,573, 6,727,095, 6,946,588, 7,022,895 and 7,439,420; and U.S. Patent Application Publication No. 2004/0209341; herein incorporated by reference in their entirety.

In some embodiments, the present invention relates to an auxotrophic requirement for an amino acid such as isoleucine in transgenic plants and plant parts. Isoleucine is an α-amino acid and has the following chemical formula: CH₃—CH₂—CH(CH₃)—CH(NH₂)—COOH. Plants can synthesize isoleucine from threonine (CH₃—CH(OH)—CH(NH₂)—COOH), and the isoleucine biosynthetic pathway includes the processing of threonine through five enzymatic steps including threonine deaminase (TD, also referred to as threonine dehydratase), acetohydroxyacid synthase (AHAS), acetohydroxyacid reductoisomerase (AHR), dihydroxy-acid dehydratase (DAD), and valine-isoleucine aminotransferase (VIAT). See, for example, Singh, ed. (1999) “Biosynthesis of valine, leucine, and isoleucine,” in Plant Amino Acids: Biochemistry and Biotechnology, pages 227-247 (Marcel Dekker). Therefore, deleting, knocking down or interfering with expression or function of any one of the enzymes in the isoleucine biosynthetic pathway results in transgenic plants or plant parts having an auxotrophic requirement for isoleucine.

Nucleic and amino acid sequences for TD, AHAS, AHR, DAD, and VIAT are known in the art. For TD, see, for example, GenBank Accession Nos. AAL57674 (Arabidopsis thaliana TD protein sequence; see GenBank Accession No. AY065037 for coding sequence); ABF98530 (Oryza sativa TD protein sequence; see GenBank Accession No. DP000009 (region: 28784851 to 28790144) for coding sequence; AAG59585 (Nicotiana attenuata TD protein sequence; see GenBank Accession No. AF229927 for coding sequence); CAA55313 (Cicer arietinum TD protein sequence; see GenBank Accession No. X78575 for coding sequence); AAA34171 (Solanum lycopersicum TD protein sequence; see GenBank Accession No. M61914 for coding sequence); SEQ ID NOS: 1-6 herein, setting forth the cDNA and protein sequences for the novel Lemna minor TD proteins disclosed herein; see also, John et al. (1995) Plant Physiol. 107(3):1023-1024; Mourad et al. (1998) Plant Physiol. 118:1534; Mourad et al. (2000) Plant Physiol. 122:619; Samach et al. (1991) Proc. Natl. Acad. Sci. U.S.A. 88(7):2678-2682; and U.S. Pat. No. 6,946,588 and U.S. Patent Application Publication No. 2004/0209341; herein incorporated by reference in their entirety.

Nucleic and amino acid sequences for AHAS are also known. See, for example, GenBank Accession Nos. AAC14572 (Hordeum vulgare AHAS (partial) protein sequence; see GenBank Accession No. AF059600 for coding sequence; ABR68866 (Solanum ptychanthum AHAS protein sequence; see GenBank Accession No. EF656478 for coding sequence); CAA87084 (Gossypium hirsutum AHAS protein sequence; see GenBank Accession No. Z46960 for coding sequence); CAA45116 (Zea mays AHAS protein sequence; see GenBank Accession No. X63553 for coding sequence); ACZ92141 (Brassica napus AHAS protein sequence; see GenBank Accession No. GU192448 for coding sequence); AAO53551 (Triticum aestivum AHAS protein sequence; see GenBank Accession No. AY210408 for coding sequence); ACU30048 (Glycine max AHAS protein sequence; see GenBank Accession No. FJ581423 for coding sequence); see also Fang et al. (1992) Plant Mol. Biol. 18:1185-1187; herein incorporated by reference in their entirety.

In addition, nucleic and amino acid sequences for AHR are known. See, for example, GenBank Accession Nos. ACU26530 (Glycine max AHR protein sequence; see GenBank Accession No. FJ594399 for coding sequence); AAL38839 (Arabidopsis thaliana AHR protein sequence; see GenBank Accession No. AY065398 for coding sequence; ACG35752 (Zea mays AHR protein sequence; see GenBank Accession No. EU963634 for coding sequence); see also Dumas et al. (1989) Biochem. J. 262:971-976; and Xu et al. (2001) Chin. Sci. Bull. 46:1808-1812; herein incorporated by reference in their entirety.

Likewise, nucleic and amino acid sequences for DAD are known. See, for example, GenBank Accession Nos. AAK64025 (Arabidopsis thaliana DAD protein sequence; see GenBank Accession No. AY039921 for coding sequence); ACU26534 (Glycine max DAD protein sequence; see GenBank Accession No. FJ594403 for coding sequence); BAD13139 (Oryza sativa DAD protein sequence; see GenBank Accession No. AP005524 for coding sequence); see also U.S. Pat. No. 6,803,223; herein incorporated by reference in their entirety.

Moreover, nucleic and amino acid sequences form VIAT are known. See, for example, GenBank Accession Nos. NP_{—}001031015 (Arabidopsis protein sequence; see GenBank Accession No. NM_{—}001035938 for coding sequence; see also Malatrasi et al. (2006) Theor. Appl. Genet. 113:965-976; and Singh and Shaner (1995) Plant Cell 7:935-944; herein incorporated by reference in their entirety.

Thus, in one embodiment, TD is the enzyme in the isoleucine biosynthetic pathway that is targeted for deletion, knockdown or interference; therefore, the compositions and methods can be directed toward isoleucine auxotrophy in transgenic plants and plant parts.

In other embodiments, the present invention relates to an auxotrophic requirement for an amino acid such as glutamine in transgenic plants and plant parts. Glutamine is an α-amino acid and has the following chemical formula: H₂N—CO—(CH₂)₂—CH(NH₂)—COOH. Plants can synthesize glutamine from glutamate (⁻OOC—(CH₂)₂—CH(NH₂)—COO), and the glutamine biosynthetic pathway includes the processing of glutamate through an enzymatic step including glutamine synthetase (GS). See, for example, Miflin and Habash (2002) J. Exp. Bot. 53:979-987. Therefore, deleting, knocking down or interfering with any one of the enzymes in the glutamine biosynthetic pathway results in a transgenic plant or plant part having an auxotrophic requirement for glutamine.

Glutamine synthetase is known in the art, and two GS isoenzymes—cytosolic (GS1) and plastidic (GS2)—have been characterized. See, for example, Cren and Hirel (1999) Plant Cell Physiol. 40:1187-1193. Nucleic and amino acid sequences for GS are known. See, for example, GenBank Accession Nos. BAA88761 (Arabidopsis thaliana GS protein sequence; see GenBank Accession No. AB015045 for coding sequence); CAB72423 (Brassica napus GS protein sequence; see GenBank Accession No. AJ271909 for coding sequence); AAF73842 (Solanum lycopersicum GS protein sequence; see GenBank Accession No. AF200360 for coding sequence); CAA71317 (Medicago truncatula GS protein sequence; see GenBank Accession No. Y10268 for coding sequence); CAA65173 (Nicotaiona tabacum GS protein sequence; see GenBank Accession No. X95932 for coding sequence); CAA46724 (Zea mays GS protein sequence; see GenBank Accession No. X65931 for coding sequence); SEQ ID NOS:7-18, setting forth the cDNA and protein sequences for the Lemna minor GS proteins disclosed herein; see also, Becker et al. (1992) Plant Mol. Biol. 19:367-379; Chen and Silflow (1996) Plant Physiol. 112:987-996; Forde and Cullimore (1989) “The molecular biology of glutamine synthetase in higher plants,” in Oxford Surveys of Plant Molecular and Cell Biology (eds. Miflin and Miflin, Oxford University Press), pages 247-296; Kim et al. (2004) J. Plant Biol. 47:401-406; Li et al. (1993) Plant Mol. Biol. 23(2):401-407; Lightfoot et al. (1988) Plant Mol. Biol. 11:191-202; Teixeira et al. (2005) J. Exp. Bot. 56:663-671; Tingey et al. (1988) J. Biol. Chem. 263:9651-9657; and U.S. Pat. Nos. 5,098,838, 5,145,777, 5,747,308, 6,329,573, and 6,727,095; herein incorporated by reference in their entirety.

Thus, in some embodiments of the invention, GS is the enzyme in the glutamine biosynthetic pathway that is targeted for deletion, knockdown, or interference; therefore, the compositions and methods of the invention can be directed toward glutamine auxotrophy in transgenic plants and plant parts. In certain embodiments, the GS enzyme is GS1; in other embodiments, the GS enzyme is GS2; in yet other embodiments, both GS1 and GS2 are targeted for deletion, knockdown, or interference.

In yet other embodiments, the present invention relates to an auxotrophic requirement for an amino acid such as histidine in transgenic plants and plant parts. The final two steps in the biosynthesis of histidine are catalyzed by the enzyme histidinol dehydrogenase (HD). In these two steps, L-histidinol is oxidized to L-histidinaldehyde and then to L-histidine via NAD-dependent oxidation reactions. Histidinol dehyrogenase activity has been detected in several plant species, including asparagus, cabbage, cucumber, egg plant, lettuce, radish, rose, squash, turnip, and wheat (see, for example, Wong and Mazalis (1981) Phytochrom. 20:1831-1834; also see U.S. Pat. No. 5,290,926; herein incorporated by reference in their entirety). Nucleic and amino acid sequences for HD are known. See, for example, GenBank Accession Nos. P24226 (Brassica oleracea var. capitata HD protein sequence; see GenBank Accession No. M60466 for coding sequence); AAN28839 (Arabidopsis thaliana HD protein sequence; see GenBank Accession No. AY143900; Q5NAY4 (Oryza sativa HD protein sequence; see GenBank Accession No. NP_{—}001042506 for reference coding sequence). Also see Nagai et al. (1991) Proc. Natl. Acad. Sci. U.S.A. 88(10):4133-4137; and U.S. Pat. No. 5,290,926. Thus, in some embodiments of the invention, HD is the enzyme in the histidine biosynthetic pathway that is targeted for deletion, knockdown, or interference; therefore, the compositions and methods of the invention can be directed toward histidine auxotrophy in transgenic plants and plant parts.

In another aspect, the present invention relates to compositions and methods for introducing and using an auxotrophic requirement for a carbohydrate in transgenic plants and plant parts. The carbohydrate can be any carbohydrate that transgenic plants or plant parts cannot make or utilize given the auxotrophic requirement. A carbohydrate is an aldehyde or ketone with many hydroxyl groups added, usually one on each carbon atom that is not part of the aldehyde or ketone functional group, and can be straight-chained or cyclic. The most basic carbohydrate unit is called a monosaccharide, e.g., glucose, fructose, galactose, xylose, and ribose. Two joined monosaccharides are called a disaccharide; examples include sucrose (glucose+fructose). Oligosaccharides and polysaccharides (for example, cellulose and starch) are composed of longer chains of monosaccharides bound together by glycosidic bonds. While oligosaccharides contain between two and nine monosaccharides, polysaccharides contain greater than ten monosaccharides. Examples of carbohydrates suitable for the auxotrophic requirement include, but are not limited to, fucose, glucose, and sucrose. See, Hassid (1969) Science 165:137-144; and Rubio et al. (2006) Plant Physiol 140: 830-843 (disclosing sucrose auxotroph due to T-DNA knockout mutants in Coenzyme A biosynthetic genes HAL3A (encoding 4′-phophopantothenoyl-cysteine decarobilase) and HAL3B (encoding gene product similar to HAL3A)). Any component within a biosynthetic pathway for a carbohydrate can be targeted at the gene or protein level to inhibit synthesis of the respective carbohydrate.

In yet another aspect, the present invention relates to compositions and methods for introducing and using an auxotrophic requirement for a fatty acid in transgenic plants and plant parts. The fatty acid can be any fatty acid that transgenic plants or plant parts cannot make or utilize given the auxotrophic requirement. A fatty acid is a carboxylic acid with a long, unbranched, aliphatic tail (chain) that can be saturated or unsaturated. In addition to saturation, fatty acids can be characterized as short, medium, or long. Short chain fatty acids (SCFA) are fatty acids with aliphatic tails of less than six carbons. Medium chain fatty acids (MCFA) are fatty acids with aliphatic tails of six to twelve carbons. Long chain fatty acids (LCFA) are fatty acids with aliphatic tails longer than twelve carbons. Very long chain fatty acids (VLCFA) are fatty acids with aliphatic tails longer than twenty-two carbons. Examples of fatty acids suitable as the auxotrophic requirement include, but are not limited to, oleic acid, palmitic acid, and stearic acid, as well as ω-3 and ω-6 fatty acids (e.g., linoleic acid and α-linolenic acid). Any component within a biosynthetic pathway for an essential fatty acid can be targeted at the gene or protein level to inhibit synthesis of the respective fatty acid. For examples of genes and encoded proteins involved in biosynthesis of essential fatty acids see, Cahoon and Shanklin (2000) Proc. Nat. Acad. Sci. USA 97:12350-12355; Volpe and Vagelos (1976) Physiol. Rev. 56:339-417; Bach et al. (2008) Proc. Nat. Acad. Sci. USA 105:14727-14731 (Arabidopsis 3-hydroxy-acyl-CoA dehydratase); Baud et al. (2004) EMBO 5:515-520 (Arabidopsis acetyl CoA carboxylase 1); Yu et al. (2004) Plant Cell Physiol. 45:503-510 (Arabidopsis lysophophatidic acid acyltransferase (LPAAT)); and U.S. Pat. No. 6,495,738; herein incorporated by reference in their entirety.

In another aspect, the present invention relates to compositions and methods for introducing and using an auxotrophic requirement for a nucleic acid in transgenic plants and plant parts. The nucleic acid can be any nucleic acid that transgenic plants or plant parts cannot make or utilize given the auxotrophic requirement. A nucleic acid is composed of three components: a nitrogenous heterocyclic base (i.e., a purine or a pyrimidine), a pentose sugar, and a phosphate group. Nucleic acids differ in the structure of the pentose sugar—deoxyribonucleic acid (DNA) contains 2-deoxyribose, while ribonucleic acid (RNA) contains ribose—thus, the difference between the two is the presence of a hydroxyl group on the ribose. Adenine, cytosine, and guanine can be found in both naturally occurring RNA and DNA, while thymine only occurs in DNA and uracil occurs only in RNA. Other rare nitrogenous bases can occur, for example, inosine in strands of mature transfer RNA. Examples of nucleic acids suitable for the auxotrophic requirement include, but are not limited to, adenine, guanine, cytosine, thymine, and uracil. Any component within a biosynthetic pathway for an essential nucleic acid can be targeted at the gene or protein level to inhibit synthesis of the respective nucleic acid. For examples of genes and encoded proteins involved in biosynthesis of essential nucleic acids see, Boldt and Zrenner (2003) Physiol. Plant 117:297-304; King et al. (1980) Planta 149:480-484; Mitsui and Ashihara (1988) Plant Cell Physiol. 29:1177-1183; Stevens et al. (1975) J. Bacteriol. 124:247-251; and Zrenner et al. (2006) Ann. Rev. Plant Biol. 57:805-836; herein incorporated by reference in their entirety.

In yet another aspect, the present invention relates to compositions and methods for introducing and using an auxotrophic requirement for a vitamin in transgenic plants and plant parts. The vitamin can be any vitamin that transgenic plants or plant parts cannot make or utilize given the auxotrophic requirement. A vitamin is an organic compound required as a nutrient in minute amounts by plants or plant parts, but excludes other essential nutrients such as dietary minerals, essential fatty acids, or essential amino acids. Vitamins are classified by their biological and chemical activity, not their structure, e.g., vitamin A, B, C, D, E, and K. Examples of vitamins suitable for the auxotrophic requirement include, but are not limited to, biotin (vitamin B7), nicotinic acid (niacin or vitamin B3), riboflavin (vitamin B2), thiamine (vitamin B1), tocopherol (vitamin E), pyridoxine (vitamin B6), and p-aminobenzoic acid (vitamin Bx). Any component within a biosynthetic pathway for an essential vitamin can be targeted at the gene or protein level to inhibit synthesis of the respective vitamin. For examples of genes and encoded proteins involved in biosynthesis of essential vitamins see Patton et al. (1998) Plant Physiol. 116:935-946; Picciocchi et al. (2001) Plant Physiol. 127:1224-1233; Pinon et al. (2005) Plant Physiol. 139:1666-1676; Shellhammer and Meinke (1990) Plant Physiol. 93:1162-1167; Woodward et al. (2010) Plant Cell 22:3305-3317 (thiamine); Rubio et al. (2006) Plant Physiol. 140:830-843 (Coenzyme A); Papini-Terzi et al. (2003) Plant Cell Physiol. 44:856-860 (thiamine); Chen and Xiong (2005) Plant Journal 44:396-408 (pyridoxine (Vitamin B6)); Wagner et al. (2006) Plant Cell (18)1722-1735 (pyridoxine (Vitamin B6)); and U.S. Pat. No. 6,849,783; herein incorporated by reference in their entirety.

In one such embodiment, the present invention relates to an auxotrophic requirement for a vitamin such as biotin in transgenic plants and plant parts. Biotin serves as a cofactor for enzymes that catalyze carboxylation, decarboxylation and transcarboxylation reactions (e.g., acetyl CoA carboxylase, 3-methylcrotonyl CoA carboxylase, propionyl CoA carboxylase and pyruvate carboxylase) in fatty acid and carbohydrate metabolism. It has the following chemical formula: C₁₀H₁₆N₂O₃S. Plants can synthesize biotin from pimeloyl-CoA, and the biotin biosynthetic pathway includes the processing of pimeloyl-CoA through four enzymatic steps including 7-keto-8-amino pelargonic acid synthase (KAPA), 7,8-diaminopelargonic acid aminotransferase (DAPA), dethiobiotin synthase (DBS), and biotin synthase (BS). See Pinon et al. (2005) Plant Physiol. 139:1666-1676. Therefore, deleting, knocking down, or interfering with any one of the enzymes in the biotin biosynthetic pathway results in transgenic plants or plant parts having an auxotrophic requirement for biotin.

Nucleic and amino acid sequences for KAPA, DAPA, DBS, and BS are known in the art. For KAPA, see, for example, GenBank Accession Nos. AAY82238 (Arabidopsis thaliana KAPA protein sequence; see GenBank Accession No. DQ017966 for coding sequence); and AAY82238; see also Pinon et al. (2005) Plant Physiol. 139:1666-1676. In addition, nucleic and amino acid sequences for DAPA are known. See, for example, GenBank Accession Nos. ABN80998 (Arabidopsis thaliana DAPA protein sequence; see GenBank Accession No. EF081156 for coding sequence). Likewise, nucleic and amino acid sequences for DBS are known. See, for example, GenBank Accession Nos. ABU50829 (Arabidopsis thaliana DBS protein sequence; see GenBank Accession No. EU090805 for coding sequence); see also Muralla et al. (2008) Plant Physiol. 146:60-73. Also see, for example, but not limited to, GenBank Accession No. ABW80569 (Arabidopsis thaliana bifunctional diaminopelargonate synthase-dethiobiotin synthetase protein sequence; see GenBank Accession No EU089963 for coding sequence); GenBank Accession No. XP_{—}002866220 (Arabidopsis lyrata subsp. lyrata bifunctional diaminopelargonate synthetase protein sequence; see NCBI Reference Sequence XM_{—}002866174.1 for coding sequence); ABU50828 (Arabidopsis thaliana diaminopelargonate synthase protein sequence; see GenBank Accession No. EU090805 for coding sequence); NP_{—}200567 (Arabidopsis thaliana adenosylmethionine-8-amino-7-oxononanoate transaminase “BIO1” protein sequence; see NCBI Reference Sequence NM_{—}125140 for coding sequence); and BAG94844 (Oryza sativa adenosylmethionine-8-amino-7-oxononanoate transaminase protein sequence; see GenBank Accession No AK100945 for coding sequence).

Moreover, nucleic and amino acid sequences for BS are known. See, for example, GenBank Accession No. AAC49445 (Arabidopsis thaliana BS protein sequence; see GenBank Accession No. U31806 for coding sequence); NP_{—}001150188 (Zea mays BS protein sequence; see GenBank Accession No. NM_{—}01156716 for coding sequence); BAD33149 (Oryza sativa BS protein sequence; see GenBank Accession No. AP004592 for coding sequence); ABB72224 (Glycine max BS protein sequence; see GenBank Accession No. DQ269214 for coding sequence); SEQ ID NOS:19-24, setting forth the cDNA and protein sequences for the Lemna minor BS proteins disclosed herein; see also, Patton et al. (1996) Plant Physiol. 112:371-378; and U.S. Pat. No. 6,849,783; herein incorporated by reference in their entirety.

Thus in one embodiment, BS is the enzyme in the biotin biosynthetic pathway that is targeted for deletion, knockdown, or interference; therefore, the compositions and methods are directed toward biotin auxotrophy in transgenic plants and plant parts.

In another aspect, the present invention relates to compositions and methods for introducing and using an auxotrophic requirement for a plant hormone (also known as plant growth substances) in transgenic plants and plant parts. Plant hormones are organic chemicals that regulate plant growth via gene expression and transcription, cell division, and growth. These signal molecules are produced within the plant at very low concentrations, and regulate cellular processes in targeted cells locally and in other locations to which they are transported. Plant hormones influence formation of flowers, fruits, seeds, stems, leaves, and roots, as well as overall plant growth and senescence. Examples of plant hormones include, but are not limited to, abscisic acid, auxins (for example, indole-3-acetic acid (IAA), indole-3-butyric acid (IBA), and 4-chloroindole-3-acetic acid (4-Cll-IAA)), cytokinins, ethylene, gibberellins, as well as other regulators of plant growth such as bassinosteroids, salicylic acid, jasmonates, plant peptide hormones, polyamines, and the like. Any component within a biosynthetic pathway for an essential plant hormone can be targeted at the gene or protein level to inhibit synthesis of the respective hormone. For examples of genes and encoded proteins involved in biosynthesis of essential plant hormones, see Blonstein et al. (1988) Mol. Gen. Genet. 215:58-64; Grennan (2006) Plant Physiol. 141:524-526; Grove et al. (1979) Nature 281:216; Haberer and Kieber (2002) Plant Physiol. 128:354-362; Kakimoto (2003) J. Plant Res. 116:233-239; Lindsey et al. (2002) Trends in Plant Science 7(2)-78-83; Margis-Pinheiro et al. (2005) Plant Cell Rep. 23:819-833; Osborne et al. (2005) Hormones, Signals and Target Cells in Plant Development (Cambridge University Press); and Sakamoto et al. (2004) Plant Physiol. 134(4):1642-1653; herein incorporated by reference in their entirety.

It is recognized that the transgenic plants or plant parts can be engineered to have more than one auxotrophic requirement, within the same or a different category of essential compounds, if so desired. For example, the growth, development, and/or survival of the transgenic plant or plant part can require external supplementation with at least one amino acid, at least one carbohydrate, at least one fatty acid, at least one nucleic acid, at least one vitamin, at least one plant hormone, or at least one precursor thereof, as well as any combination thereof. In other embodiments, the growth, development, and/or survival of the transgenic plant or plant part can require external supplementation with an amino acid and a carbohydrate, an amino acid and a fatty acid, an amino acid and a nucleic acid, an amino acid and a vitamin, an amino acid and a plant hormone, a carbohydrate and a fatty acid, a carbohydrate and a nucleic acid, a carbohydrate and a vitamin, a carbohydrate and a plant hormone, a fatty acid and a nucleic acid, a fatty acid and a vitamin, a fatty acid and a plant hormone, a nucleic acid and a vitamin, a nucleic acid and a plant hormone, or a vitamin and a plant hormone.

In other aspects, the present invention relates to methods of biocontaining a transgenic plant or plant part having at least one auxotrophic requirement. A transgenic plant or plant part having a heterologous polynucleotide of interest therein, for example, a polynucleotide comprising a transgene encoding a polypeptide of interest, or a polynucleotide construct of interest, can be rendered auxotrophic for an essential compound by any means known in the art such that development, growth and/or survival of the transgenic plant or plant part will be conditioned upon exogenous supplementation of the essential compound. For example, if the transgenic plant or plant part has an auxotrophic requirement for an amino acid, then growth, development or survival of the plant depends upon exogenous supplementation of that amino acid.

In the absence of this amino acid, the transgenic plant or plant part cannot grow, develop, and/or survive and thus is effectively biocontained. In yet other aspects, the present invention relates to methods of using transgenic plants or plants part having at least one auxotrophic requirement to produce recombinant polypeptides. Plants or plant parts having a heterologous polynucleotide or transgene encoding a polypeptide of interest can be rendered auxotrophic for an essential compound such that production of the polypeptide of interest will be conditioned upon exogenous supplementation of the essential compound. For example, if the transgenic plants or plant parts have an auxotrophic requirement for an amino acid, then growth of the plants or plant parts and production of the polypeptide of interest depends upon exogenous supplementation of that amino acid. In the absence of the essential amino acid, the transgenic plants or plant parts cannot survive and thus cannot produce the recombinant polypeptide of interest.

The compositions and methods of the invention find use in maintaining biodiversity and protecting the ecosystem. Because the transgenic plants and plant parts are auxotrophic, they require an exogenously supplied essential compound, which typically is not available to it in sufficient amounts outside the laboratory or in the absence of human intervention. Thus, when these transgenic plants or plant parts are not provided the essential compound or are disposed of, their ability to survive and transfer transgenes to conventional crops or wild-type plant populations is attenuated.

Novel Polynucleotides and Polypeptides for Introducing an Auxotrophic Requirement into Transgenic Plants or Plant Parts

The present invention provides novel compositions for introducing an auxotrophic requirement into a transgenic plant or plant part thereof, more particularly novel polynucleotides encoding components of biosynthetic pathways involved in production of the amino acids isoleucine and glutamine and the vitamin biotin. The novel polynucleotides of the invention encode plant-derived threonine deaminase (TD), glutamine synthetase (GS), and biotin synthase (BS) proteins, and variants and fragments thereof. Inhibitory polynucleotide constructs based on these novel TD, GS, and BS coding sequences advantageously can be used to introduce an auxotrophic requirement into transgenic plants and plant parts, more specifically, a requirement for exogenous supplementation with isoleucine, glutamine, and/or biotin in order to support growth, development, and survival of the transgenic plant or plant part.

In this manner, the present invention provides novel isolated polynucleotide and polypeptide sequences for threonine deaminase (TD), cytosol-localized glutamine synthetase (GS1), plastid-localized glutamine synthetase (GS2), and biotin synthase (BS) isolated from Lemna minor, a member of the duckweed family, and variants and fragments of these polynucleotides and polypeptides. Inhibition of the expression or function of these proteins, or biologically active variants or fragments thereof, allows for introduction of an auxotrophic requirement into a transgenic plant or plant part thereof.

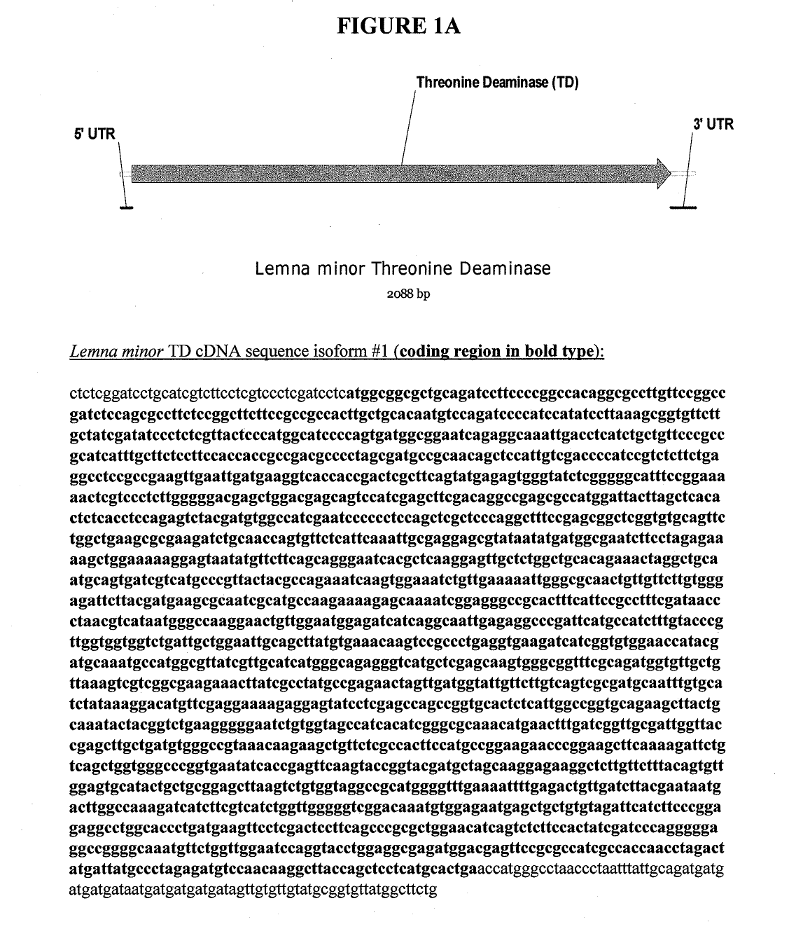

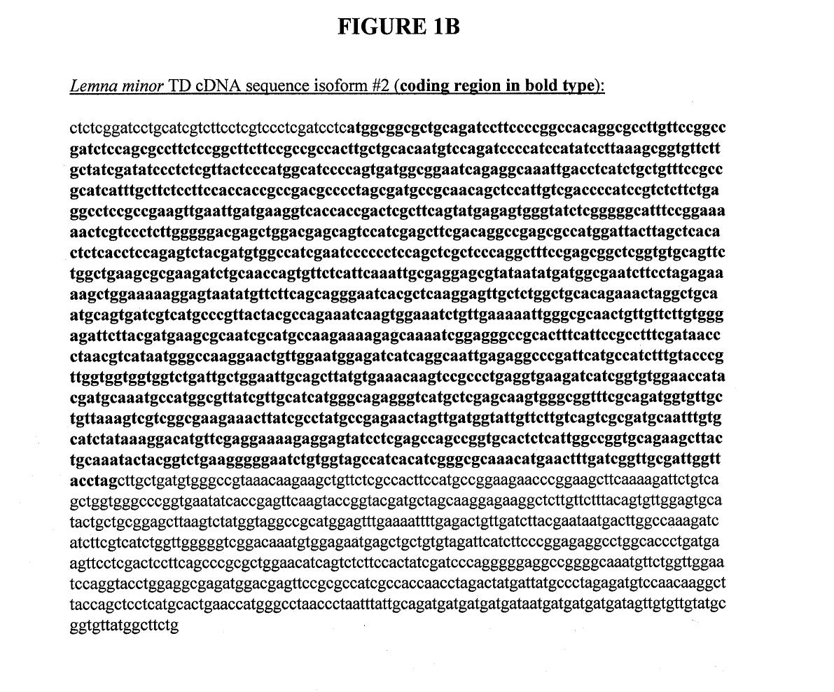

The full-length cDNA sequence (2088 nt in length), including 5′- and 3′-UTR, for L. minor TD isoform #1 is set forth in FIG. 1A; see also SEQ ID NO:1 (open reading frame (ORF) set forth in SEQ ID NO:2). The predicted amino acid sequence (652 aa) encoded thereby is set forth in SEQ ID NO:3. The full-length cDNA sequence (2091 nt in length), including 5′- and 3′-UTR, for L. minor TD isoform #2 is set forth in FIG. 1B; see also SEQ ID NO:4 (open reading frame set forth in SEQ ID NO:5). The premature stop codon at position 1445 of SEQ ID NO:4 results in an encoded truncated protein having the predicted amino acid sequence (468 aa) set forth in SEQ ID NO:6. These two L. minor TD isoforms share 99.7% sequence identity at the nucleotide level. The encoded TD isoform #1 and TD isoform #2 proteins share 99.6% sequence identity in the region of overlap. The L. minor TD cDNAs and encoded proteins share some similarity with other threonine deaminases from other higher plants. For example, the L. minor TD isoform #1 shares approximately 67%, 71%, and 56% amino acid sequence identity with TD proteins from Arabidopsis thaliana (GenBank Accession No. AAL57674), Oryza sativa (GenBank Accession No. ABF98530), and Nicotlana attenuata (GenBank Accession No. AAG59585), respectively.

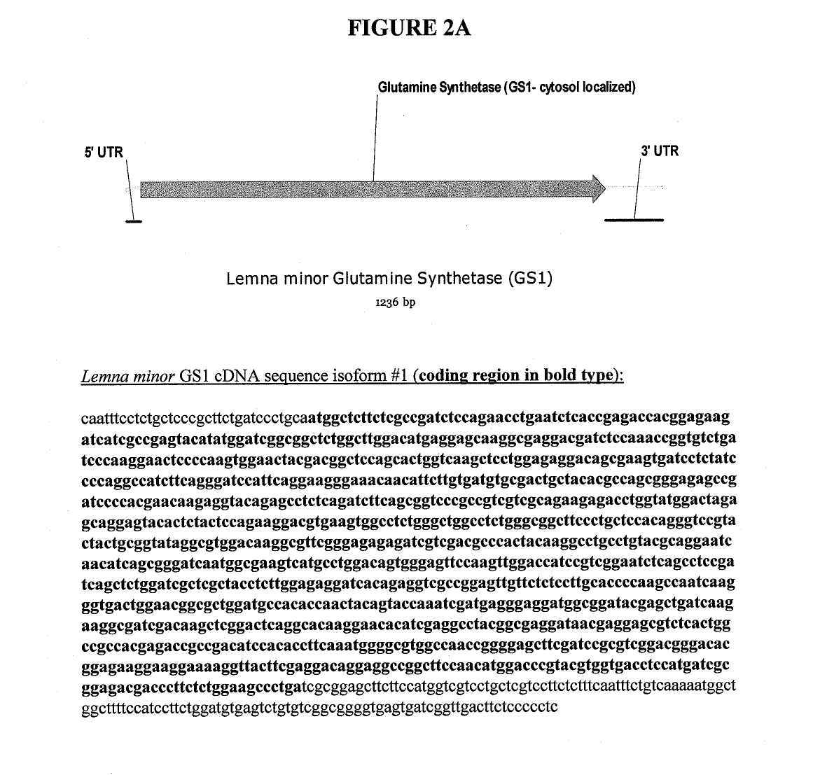

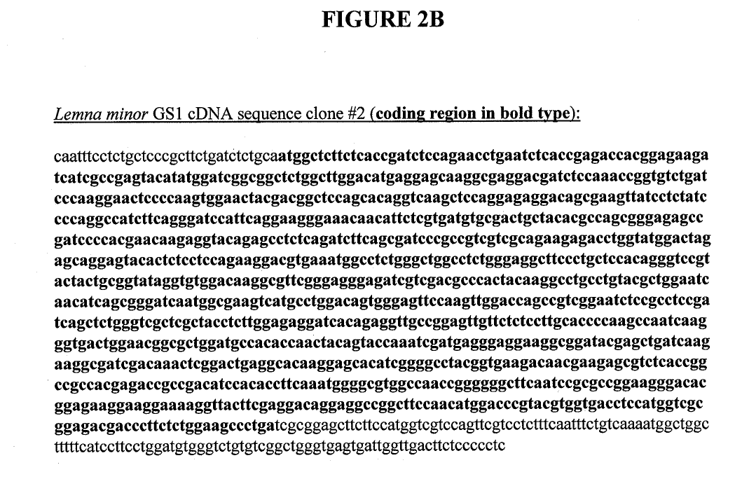

The present invention also provides novel sequences for a cytosolic-localized glutamine synthetase (GS1) isolated from Lemna minor. The full-length cDNA sequence (1236 nt in length), including 5′- and 3′-UTR, for L. minor glutamine synthetase 1 (GS1) isoform #1, a cytosol localized enzyme, is set forth in FIG. 2A; see also SEQ ID NO:4 (ORF set forth in SEQ ID NO:5). The predicted amino acid sequence (356 aa) encoded thereby is set forth in SEQ ID NO:6. The full-length cDNA sequence (1233 nt in length), including 5′- and 3′-UTR, for L. minor glutamine synthetase 1 (GS1) isoform #2, also a cytosol localized enzyme, is set forth in FIG. 2B; see also SEQ ID NO:10 (ORF set forth in SEQ ID NO:11). The predicted amino acid sequence (356 aa) encoded thereby is set forth in SEQ ID NO:12. These two L. minor GS1 isoforms share 96.5% and 97.8% identity at the nucleotide and protein levels, respectively. The encoded GS1 protein shares some similarity with other GS1 proteins from other plants. For example, the L. minor GS1 protein shares approximately 86%, 86%, and 85% sequence identity with the glutamine synthetase proteins from Camellia sinensis (GenBank Accession No. BAD99525), Lotus japonicus (GenBank Accession No. CAA73366), and Vitis vinifera (GenBank Accession No. P51119), respectively.

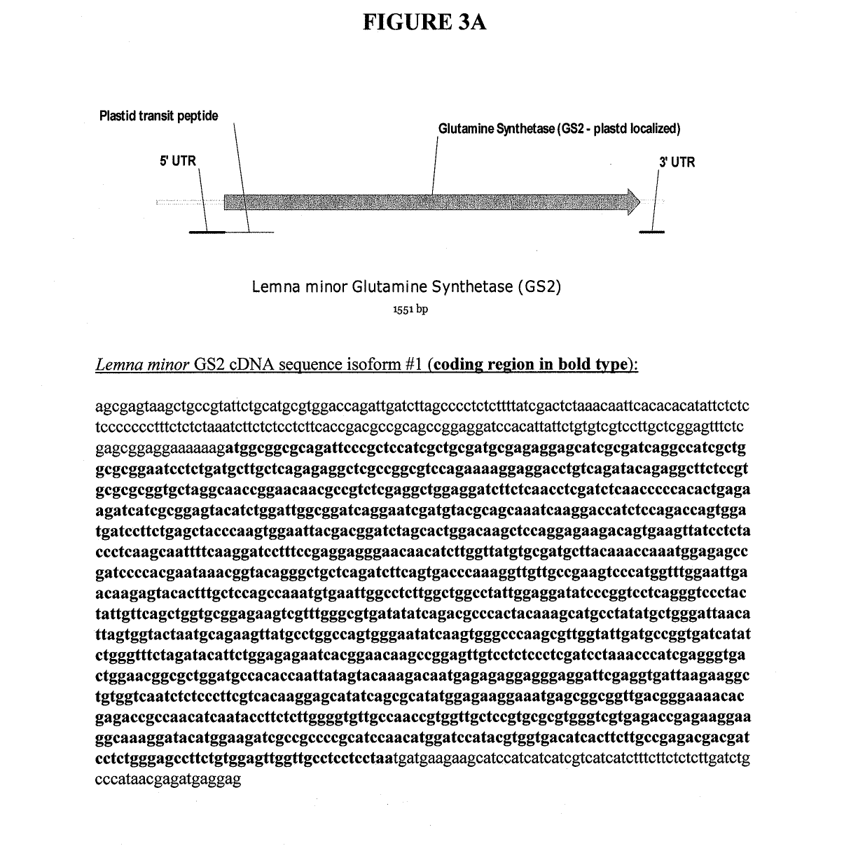

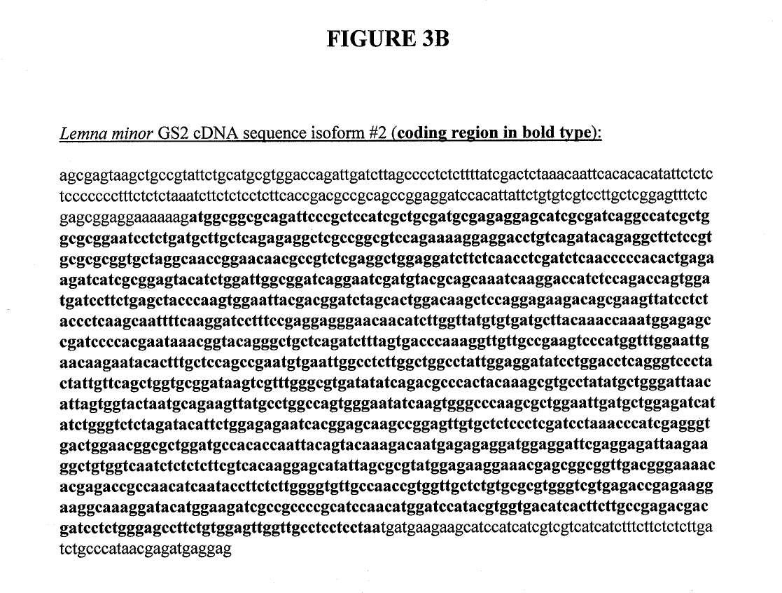

The present invention also provides novel sequences for a plastid-localized glutamine synthetase (GS2) isolated from Lemna minor. The full-length cDNA sequence (1551 nt in length), including 5′- and 3′-UTR, for L. minor glutamine synthetase 1(GS2) isoform #1 is set forth in FIG. 3A; see also SEQ ID NO:13 (ORF set forth in SEQ ID NO:14). The predicted amino acid sequence (424 aa) encoded thereby is set forth in SEQ ID NO:15. The full-length cDNA sequence (1275 nt in length), including 5′- and 3′-UTR, for L. minor glutamine synthetase 1(GS2) isoform #2 is set forth in FIG. 3B; see also SEQ ID NO:16 (ORF set forth in SEQ ID NO:17). The predicted amino acid sequence (424 aa) encoded thereby is set forth in SEQ ID NO:18. These two L. minor GS2 isoforms share 98.4% and 99.1% identity at the nucleotide and protein levels, respectively. The encoded GS2 proteins share some similarity with GS2 proteins from other plants. For example, the L. minor GS2 isoform #1 protein shares approximately 80%, 79%, and 79% sequence identity with the plastid localized glutamine synthetase proteins from Vigna radiate (GenBank Accession No. ADK27329), Avicennia marina (GenBank Accession No. BAF62340), and Phaseolus vulgaris (GenBank Accession No. P15102), respectively.

The percent identities between the four L. minor GS cDNAs and the predicted amino acid sequences encoded thereby are shown in Table 11. As expected, the GS1 sequences share greater identity with each other, but still share at least 70% identity at the nucleotide level, and at least 79% identity at the amino acid level.

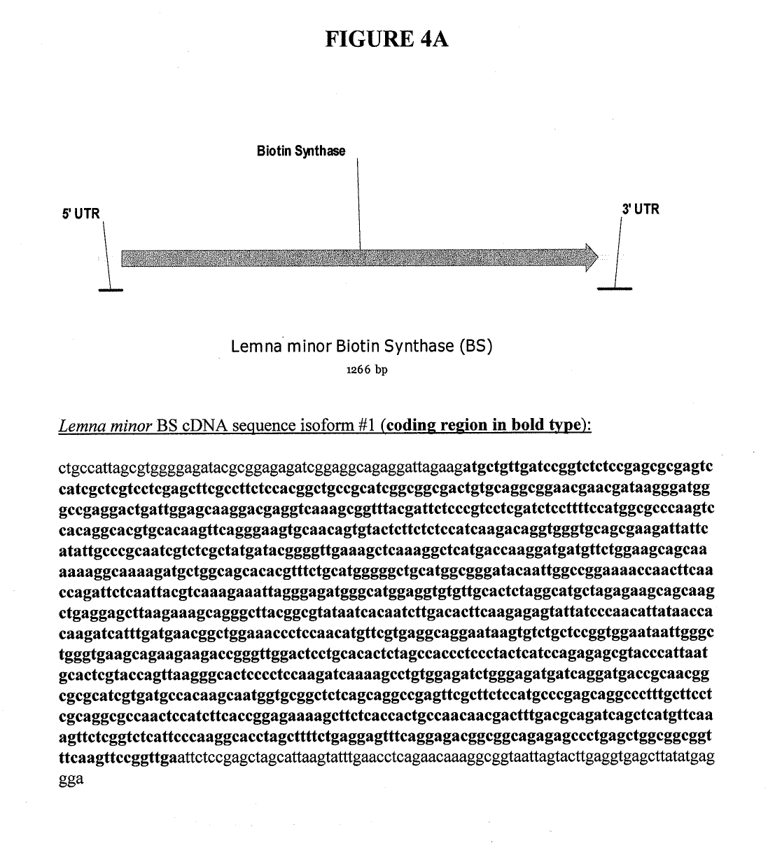

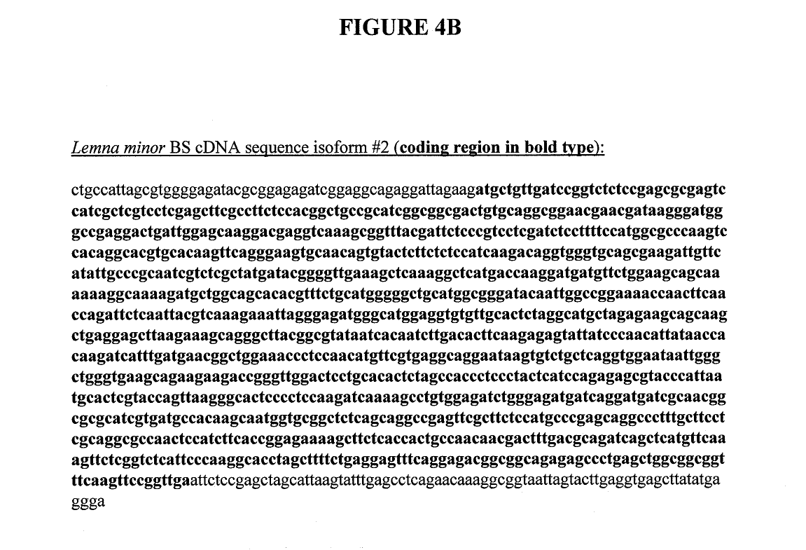

The present invention also provides novel sequences for a biotin synthase (BS) isolated from Lemna minor. The full-length cDNA sequence (1266 nt in length), including 5′- and 3′-UTR, for L. minor BS isoform #1 is set forth in FIG. 4A; see also SEQ ID NO:19 (ORF set forth in SEQ ID NO:20). The predicted amino acid sequence (377 aa) encoded thereby is set forth in SEQ ID NO:21. The full-length cDNA sequence (1266 nt in length), including 5′- and 3′-UTR, for L. minor BS isoform #2 is set forth in FIG. 4B; see also SEQ ID NO:22 (ORF set forth in SEQ ID NO:23). The predicted amino acid sequence (377 aa) encoded thereby is set forth in SEQ ID NO:24. These two isoforms share 99.7% and 99.5% identity at the nucleotide and protein levels, respectively The encoded BS protein shares some similarity with other BS proteins from other plants. For example, the L. minor BS protein isoforms #1 shares approximately 82%, 82%, 80%, and 79% sequence identity with the biotin synthase proteins from Zea mays (GenBank Accession No. NP_{—}001150188), Brassica rapa (GenBank Accession No. ABI63585), Arabidopsis thaliana (GenBank Accession No. NP_{—}181864), and Ricinus communis (GenBank Accession No. XP_{—}002529753), respectively.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When a protein of the invention or biologically active portion thereof is recombinantly produced, optimally culture medium represents less than about 30%, 20%, 10%, 5%, or 1% (by dry weight) of chemical precursors or non-protein-of-interest chemicals.

The coding sequence for the L. minor TD isoform #1 gene is set forth as nucleotides (nt) 41-1999 of SEQ ID NO:1 and as SEQ ID NO:2, and the amino acid sequence for the encoded polypeptide is set forth in SEQ ID NO:3. The coding sequence for the L. minor TD isoform #2 gene is set forth as nucleotides 41-1447 of SEQ ID NO:4 and as SEQ ID NO:5, and the amino acid sequence for the encoded TD polypeptide is set forth in SEQ ID NO:6. The coding sequence for the L. minor GS1 isoform #1 gene is set forth as nucleotides 34-1104 of SEQ ID NO:7 and as SEQ ID NO:8, and the amino acid sequence for the encoded TD polypeptide is set forth in SEQ ID NO:9. The coding sequence for the L. minor GS1 isoform #2 gene is set forth as nucleotides 34-1104 of SEQ ID NO:10 and as SEQ ID NO:11, and the amino acid sequence for the encoded GS1 polypeptide is set forth in SEQ ID NO:12. The coding sequence for the L. minor GS2 isoform #1 gene is set forth as nucleotides 205-1479 of SEQ ID NO:13 and as SEQ ID NO:14, and the amino acid sequence for the encoded GS2 polypeptide is set forth in SEQ ID NO:15. The coding sequence for the L. minor GS2 isoform #2 gene is set forth as nucleotides 205-1479 of SEQ ID NO:16 and as SEQ ID NO:17, and the amino acid sequence for the encoded GS2 polypeptide is set forth in SEQ ID NO:18. The coding sequence for the L. minor BS isoform #1 gene is set forth as nucleotides 54-1187 of SEQ ID NO:19 and as SEQ ID NO:20, and the amino acid sequence for the encoded BS polypeptide is set forth as SEQ ID NO:21. The coding sequence for the L. minor BS isoform #2 gene is set forth as nucleotides 54-1187 of SEQ ID NO:22 and as SEQ ID NO:23, and the amino acid sequence for the encoded BS polypeptide is set forth as SEQ ID NO:24.

In particular, the present invention provides for isolated polynucleotides comprising nucleotide sequences encoding the amino acid sequences shown in SEQ ID NOS:3, 6, 9, 12, 15, 18, 21, and 24. Further provided are polypeptides having an amino acid sequence encoded by a polynucleotide described herein, for example those polynucleotides set forth in SEQ ID NOS:1, 2, 4, 5, 7, 8, 10, 11, 13, 14, 16, 17, 19, 20, 22, and 23, and fragments and variants thereof. Nucleic acid molecules comprising the complements of these nucleotide sequences are also provided. It is recognized that the coding sequence for the TD, GS1, GS2, and/or BS gene can be expressed in a plant for overexpression of the encoded TD, GS1, GS2, and/or BS protein. However, for purposes of suppressing or inhibiting the expression of these proteins, the respective nucleotide sequences of SEQ ID NOs: 1, 2, 4, 5, 7, 8, 10, 11, 13, 14, 16, 17, 19, 20, 22, and 23 will be used to design constructs for suppression of expression of the respective TD, GS1, GS2, and/or BS protein. Thus, polynucleotides, in the context of suppressing the TD protein refers to the TD coding sequences and to polynucleotides that when expressed suppress or inhibit expression of the TD gene, for example, via direct or indirect suppression as noted herein below. Similarly, polynucleotides, in the context of suppressing or inhibiting the GS1 or GS2 protein refers to the GS1 or GS2 coding sequences and to polynucleotides that when expressed suppress or inhibit expression of the GS1 or GS2 gene, for example, via direct or indirect suppression as noted herein below. In like manner, polynucleotides, in the context of suppressing or inhibiting the BS protein refers to the BS coding sequences and to polynucleotides that when expressed suppress or inhibit expression of the BS gene, for example, via direct or indirect suppression as noted herein below.

Fragments and variants of the disclosed polynucleotides and proteins encoded thereby are also encompassed by the present invention. By “fragment” is intended a portion of the TD, GS1, GS2, or BS polynucleotide or a portion of the TD, GS1, GS2, or BS amino acid sequence encoded thereby. Fragments of a polynucleotide may encode protein fragments that retain the biological activity of the native protein and hence have TD, GS1, GS2, or BS activity as noted elsewhere herein. Alternatively, fragments of a polynucleotide that are useful as hybridization probes generally do not encode fragment proteins retaining biological activity. Fragments of a TD, GS1, GS2, or BS polynucleotide can also be used to design inhibitory sequences for suppression of expression of the TD, GS1, GS2, and/or BS polypeptide. Thus, for example, fragments of a nucleotide sequence may range from at least about 15 nucleotides, about 20 nucleotides, about 25 nucleotides, about 30 nucleotides, about 35 nucleotides, about 40 nucleotides, about 45 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 225 nucleotides, about 250 nucleotides, about 275 nucleotides, about 300 nucleotides, about 325 nucleotides, about 350 nucleotides, about 375 nucleotides, about 400 nucleotides, about 425 nucleotides, about 450 nucleotides, about 475 nucleotides, about 500 nucleotides, about 525 nucleotides, about 550 nucleotides, about 575 nucleotides, about 600 nucleotides, about 625 nucleotides, about 650 nucleotides, about 700 nucleotides, about 725 nucleotides, about 750 nucleotides, about 775 nucleotides, about 800 nucleotides, about 825 nucleotides, about 850 nucleotides, about 875 nucleotides, about 900 nucleotides, about 925 nucleotides, about 950 nucleotides, about 975 nucleotides, about 1000 nucleotides, about 1025 nucleotides, about 1050 nucleotides, and up to the full-length polynucleotide encoding the proteins of the invention (i.e., up to 2088, 1959, 2091, 1407, 1236, 1071, 1233, 1071, 1551, 1275, 1551, 1275, 1266, 1134, 1266, or 1134 nucleotides of SEQ ID NO:1, 2, 4, 5, 7, 8, 10, 11, 13, 14, 16, 17, 19, 20, 22, or 23, respectively).

A fragment of a TD polynucleotide that encodes a biologically active portion of a TD protein of the invention will encode at least 15, 25, 30, 50, 100, 150, 200, 250, 300, 350, 400, 450, 475, 500, 525, 550, 575, 600, 625, 650 contiguous amino acids, or up to the total number of amino acids present in a full-length TD protein of the invention (for example, up to 652 amino acids or up to 468 amino acids for SEQ ID NO:3 or SEQ ID NO:6, respectively). A fragment of a GS1 polynucleotide that encodes a biologically active portion of a full-length GS1 protein of the invention will encode at least 15, 25, 30, 50, 100, 150, 200, 250, 300, 350 contiguous amino acids, or up to the total number of amino acids present in a full-length GS1 protein of the invention (for example, 356 amino acids for SEQ ID NO:9 or SEQ ID NO: 12). A fragment of a GS2 polynucleotide that encodes a biologically active portion of a GS2 protein of the invention will encode at least 15, 25, 30, 50, 100, 150, 200, 250, 300, 350, 400 contiguous amino acids, or up to the total number of amino acids present in a GS2 protein of the invention (for example, 424 amino acids for SEQ ID NO:15 or SEQ ID NO: 18). A fragment of a BS polynucleotide that encodes a biologically active portion of a full-length BS protein of the invention will encode at least 15, 25, 30, 50, 100, 150, 200, 250, 300, 350 contiguous amino acids, or up to the total number of amino acids present in a full-length BS protein of the invention (for example, 377 amino acids for SEQ ID NO:21 or SEQ ID NO:24).

Thus, a fragment of a TD, GS1, GS2, or BS polynucleotide may encode a biologically active portion of a TD, GS1, GS2, or BS protein, respectively, or it may be a fragment that can be used as a hybridization probe or PCR primer, or used to design inhibitory sequences for suppression, using methods disclosed below. A biologically active portion of a TD, GS1, GS2, or BS protein can be prepared by isolating a portion of one of the TD, GS1, GS2, or BS polynucleotides of the invention, respectively, expressing the encoded portion of the TD, GS1, GS2, or BS protein (e.g., by recombinant expression in vitro), and assessing the activity of the encoded portion of the TD, GS1, GS2, or BS polypeptide. Polynucleotides that are fragments of an TD, GS1, GS2, or BS nucleotide sequence comprise at least 15, 20, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1125, 1250, 1275, 1,300, 1325, 1350, 1375, 1,400, 1425, or 1450 contiguous nucleotides, or up to the number of nucleotides present in a TD, GS1, GS2, or BS polynucleotide disclosed herein (for example, up to 2088, 1959, 2091, 1407, 1236, 1071, 1233, 1071, 1551, 1275, 1551, 1275, 1266, 1134, 1266, or 1134 nucleotides of SEQ ID NO:1, 2, 4, 5, 7, 8, 10, 11, 13, 14, 16, 17, 19, 20, 22, or 23, respectively).

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the TD, GS1, GS2, or BS polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a TD, GS1, GS2, or BS protein of the invention. Generally, variants of a particular polynucleotide of the invention (for example, SEQ ID NO:1, 2, 4, 5, 7, 8, 10, 11, 13, 14, 16, 17, 19, 20, 22, or 2, fragments thereof and complements of these sequences) will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the TD, GS1, GS2, or BS polypeptide of SEQ ID NO:3 or 6, SEQ ID NO:9 or 12, SEQ ID NO:15 or 18, or SEQ ID NO:21 or 24, respectively, is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, the threonine deaminase, glutamine synthetase, or biotin synthase activity of the disclosed L. minor TD, GS1, GS2, or BS proteins of the invention. Such variants may result from, for example, genetic polymorphism or from human manipulation. Biologically active variants of a native TD, GS1, GS2, or BS protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants and fragments of the TD, GS1, GS2, and BS proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the polynucleotides of the invention include both the naturally occurring TD, GS1, GS2, and BS sequences as well as mutant forms. Likewise, the proteins of the invention encompass both naturally occurring TD, GS1, GS2, and BS proteins as well as variations and modified forms thereof. Such variants will continue to possess the desired activity. Thus, where expression of a functional protein is desired, the expressed protein will possess the desired TD, GS1, GS2, or BS activity. Where the objective is inhibition of expression or function of the TD, GS1, GS2, or BS polypeptide, in order to render a plant or plant part auxotrophic, the desired activity of the variant polynucleotide or polypeptide is one of inhibiting expression or function of the respective TD, GS1, GS2, and/or BS polypeptide. Obviously, where expression of a functional TD, GS1, GS2, or BS variant is desired, the mutations that will be made in the DNA encoding the variant must not place the sequence out of reading frame and optimally will not create complementary regions that could produce secondary mRNA structure. See, EP Patent Application Publication No. 75,444.

Where a functional protein is desired, the deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays, including the assays for monitoring TD, GS1, GS2, or BS activity described herein below in the Experimental section.

Variant polynucleotides and proteins also encompass sequences and proteins derived from a mutagenic and recombinogenic procedure such as DNA shuffling. With such a procedure, one or more different TD, GS1, GS2, or BS coding sequences can be manipulated to create a new TD, GS1, GS2, or BS protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the TD, GS1, GS2, or BS gene of the invention and other known TD, GS1, GS2, or BS genes, respectively, to obtain a new gene coding for a protein with an improved property of interest. Strategies for such DNA shuffling are known in the art. See, for example, Stemmer (1994) Proc. Natl. Acad Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The comparison of sequences and determination of percent identity and percent similarity between two sequences can be accomplished using a mathematical algorithm. In a preferred embodiment, the percent identity between two amino acid sequences is determined using the Needleman and Wunsch (1970) J. Mol. Biol. 48:444-453 algorithm, which is incorporated into the GAP program in the GCG software package (available at www.accelrys.com), using either a BLOSSUM62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a BLOSUM62 scoring matrix (see Henikoff et al. (1989) Proc. Natl. Acad. Sci. USA 89:10915) and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity limitation of the invention) is using a BLOSUM62 scoring matrix with a gap weight of 60 and a length weight of 3.

The percent identity between two amino acid or nucleotide sequences can also be determined using the algorithm of Meyers and Miller (1989) CABIOS 4:11-17 which has been incorporated into the ALIGN program (version 2.0), using a PAM120 weight residue table, a gap length penalty of 12 and a gap penalty of 4.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al. (2001) Molecular Cloning: A Laboratory Manual (Cold Spring Harbor Laboratory Press, Cold Spring Harbor, N.Y.) and Tijssen (1993) Hybridization With Nucleic Acid Probes, Part I: Theory and Nucleic Acid Preparation (Laboratory Techniques in Biochemistry and Molecular Biology, Elsevier Science Ltd., NY, N.Y.).

For purposes of the present invention, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

The TD, GS1, GS2, and BS polynucleotides of the invention can be used as probes for the isolation of corresponding homologous sequences in other plant species. In this manner, methods such as PCR, hybridization, and the like can be used to identify such sequences based on their sequence homology to the sequences of the invention. See, for example, Sambrook et al. (1989) Molecular Cloning: A Laboratory Manual (2d ed., Cold Spring Harbor Laboratory Press, Plainview, N.Y.) and Innis et al. (1990), PCR Protocols: A Guide to Methods and Applications (Academic Press, New York). Polynucleotide sequences isolated based on their sequence identity to the entire TD, GS1, GS2, or BS polynucleotides of the invention (i.e., SEQ ID NOS:1, 2, 4, and 5 for TD; SEQ ID NOS:7, 8, 10, and 11 for GS1; SEQ ID NOS:13, 14, 16, and 17 for GS2; and SEQ ID NOS:19, 20, 22, and 23 for BS) or to fragments and variants thereof are encompassed by the present invention.

In a PCR method, oligonucleotides primers can be designed for use in PCR reactions for amplification of corresponding DNA sequences from cDNA or genomic DNA extracted from any plant of interest. Known methods of PCR include, but are not limited to, methods using paired primers, nested primers, single specific primers, degenerate primers, gene-specific primers, vector-specific primers, partially-mismatched primers, and the like. Methods for designing PCR primers and PCR cloning are generally known in the art and are disclosed in Sambrook et al. (1989) Molecular Cloning. A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In a hybridization method, all or part of a known nucleotide sequence can be used as a probe that selectively hybridizes to other corresponding polynucleotides present in a population of cloned genomic DNA fragments or cDNA fragments (i.e., cDNA or genomic libraries) from another plant of interest. The so-called hybridization probes may be genomic DNA fragments, cDNA fragments, RNA fragments, or other oligonucleotides, and may be labeled with a detectable group such as ³²P, or any other detectable marker. Probes for hybridization can be made by labeling synthetic oligonucleotides based on the nucleotide sequence of interest, for example, the TD, GS1, GS2, or BS polynucleotides of the invention. Degenerate primers designed on the basis of conserved nucleotides or amino acid residues in the known nucleotide or encoded amino acid sequence can additionally be used. Methods for construction of cDNA and genomic libraries, and for preparing hybridization probes, are generally known in the art and are disclosed in Sambrook et al. (1989) Molecular Cloning: A Laboratory Manual (2d ed., Cold Spring Harbor Laboratory Press, Plainview, N.Y.), herein incorporated by reference.

For example, all or part of the specific known TD, GS1, GS2, or BS polynucleotide sequence may be used as a probe that selectively hybridizes to other TD, GS1, GS2, or BS nucleotide and messenger RNAs, respectively. To achieve specific hybridization under a variety of conditions, such probes include sequences that are unique and are preferably at least about 10 nucleotides in length, and more optimally at least about 20 nucleotides in length. This technique may be used to isolate other corresponding TD, GS1, GS2, or BS nucleotide sequences from a desired plant species or as a diagnostic assay to determine the presence of a TD, GS1, GS2, or BS coding sequences in a plant species of interest. Hybridization techniques include hybridization screening of plated DNA libraries (either plaques or colonies; see, for example, Innis et al., eds. (1990) PCR Protocols: A Guide to Methods and Applications (Academic Press, New York)).

Thus, in addition to the native TD, GS1, GS2, and BS polynucleotides and fragments and variants thereof, the isolated polynucleotides of the invention also encompass homologous DNA sequences identified and isolated from other plant species by hybridization with entire or partial sequences obtained from the TD, GS1, GS2, or BS polynucleotides of the invention or variants thereof. Conditions that will permit other DNA sequences to hybridize to the DNA sequences disclosed herein can be determined in accordance with techniques generally known in the art. For example, hybridization of such sequences may be carried out under various conditions of moderate, medium, high, or very high stringency as noted herein above. Identification of homologous TD, GS1, GS2, or BS polynucleotides in other plant species of interest may allow for the design of species-specific inhibitory constructs for introducing an auxotrophic requirement for isoleucine, glutamine, and/or biotin into a given plant species of interest.

### Methods for Introducing an Auxotrophic Requirement and Biocontaining Transgenic Plants and Plant Parts

The present invention provides methods and compositions for introducing and using an auxotrophic requirement to biocontain transgenic plants and plant parts. The term “introducing” in the context of an auxotrophic requirement is intended to mean the manipulation of the transgenic plant or plant part, either by way of mutation or introduction of an inhibitory polynucleotide construct, such that expression or function of a component of one or more biosynthetic pathways for one or more essential compounds, for example, an amino acid, fatty acid, carbohydrate, nucleic acid, vitamin, plant hormone, or precursor thereof, is inhibited. The auxotrophic requirement can be introduced into a plant or plant part that is already transgenic, as defined herein above. Alternatively, the auxotrophic requirement can be introduced into a wild-type plant or plant part, and the resulting wild-type plant or plant part having the auxotrophic requirement can then be made transgenic for any additional heterologous polynucleotide sequence of interest. In yet other embodiments, the auxotrophic requirement and transgenic status of the plant or plant part can be introduced simultaneously, for example, by introducing a single polynucleotide construct comprising a heterologous polynucleotide sequence that confers a trait of interest and a heterologous polynucleotide sequence that confers the auxotrophic requirement, or by introducing at least two polynucleotide constructs, one of which comprises a heterologous polynucleotide sequence that confers a trait of interest, and the other of which comprises a heterologous polynucleotide sequence that confers the auxotrophic requirement.

The term “introducing” in the context of a polynucleotide, for example, a heterologous polynucleotide of interest or an inhibitory polynucleotide construct, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host plant cell of interest in a single transformation event, in separate transformation events, or, for example, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a polynucleotide construct described herein, introduced into a plant integrates into the genome of the plant and is capable of being inherited by the progeny thereof, more particularly, by the progeny of multiple successive generations. In some embodiments, successive generations include progeny produced vegetatively (i.e., asexual reproduction), for example, with clonal propagation. In other embodiments, successive generations include progeny produced via sexual reproduction. A plant host that is “stably transformed” with at least one heterologous polynucleotide of interest (for example, a heterologous polynucleotide that encodes a protein of interest, or an inhibitory polynucleotide that targets expression and/or function of a protein of interest) refers to a plant host that has the heterologous polynucleotide(s) integrated into its genome, and is capable of producing progeny, either via asexual or sexual reproduction, that also comprise the heterologous polynucleotide(s) stably integrated into their genome, and hence the progeny will also exhibit the desired phenotype conferred by the heterologous polynucleotide.

As used herein, the term “plant” includes reference to whole plants, plant organs (e.g., leaves, stems, roots, etc.), seeds, plant cells, and progeny of same. Parts of transgenic plants are to be understood within the scope of the invention to comprise, for example, plant cells, protoplasts, tissues, callus, embryos as well as flowers, ovules, stems, fruits, leaves, roots, root tips, and the like originating in transgenic plants or their progeny previously transformed with a DNA molecule of the invention and therefore consisting at least in part of transgenic cells. As used herein, the term “plant cell” includes, without limitation, cells of seeds, embryos, meristematic regions, callus tissue, leaves, roots, shoots, gametophytes, sporophytes, pollen, and microspores.

In some embodiments, the auxotrophic requirement is introduced into the transgenic plant or plant part by introducing a polynucleotide construct comprising a nucleotide sequence that inhibits expression or function of a component of a biosynthetic pathway for an essential compound in the transgenic plant or plant part thereof. The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The terms “inhibit,” “inhibition,” and “inhibiting” as used herein refer to any decrease in the expression or function of a target gene product, including any relative decrement in expression or function up to and including complete abrogation of expression or function of the target gene product. The term “expression” as used herein in the context of a gene product refers to the biosynthesis of that gene product, including the transcription and/or translation and/or assembly of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product.

The term “inhibitory sequence” encompasses any polynucleotide or polypeptide sequence that is capable of inhibiting the expression of a target gene product, for example, at the level of transcription or translation, or which is capable of inhibiting the function of a target gene product. Examples of inhibitory sequences include, but are not limited to, full-length polynucleotide or polypeptide sequences, truncated polynucleotide or polypeptide sequences, fragments of polynucleotide or polypeptide sequences, variants of polynucleotide or polypeptide sequences, sense-oriented nucleotide sequences, antisense-oriented nucleotide sequences, the complement of a sense- or antisense-oriented nucleotide sequence, inverted regions of nucleotide sequences, hairpins of nucleotide sequences, double-stranded nucleotide sequences, single-stranded nucleotide sequences, combinations thereof, and the like. The term “polynucleotide sequence” includes sequences of RNA, DNA, chemically modified nucleic acids, nucleic acid analogs, combinations thereof, and the like.

It is recognized that inhibitory polynucleotides include nucleotide sequences that directly (i.e., do not require transcription) or indirectly (i.e., require transcription or transcription and translation) inhibit expression of a target gene product. For example, an inhibitory polynucleotide can comprise a nucleotide sequence that is a chemically synthesized or in vitro-produced small interfering RNA (siRNA) or micro RNA (miRNA) that, when introduced into a plant cell, tissue, or organ, would directly, though transiently, silence expression of the target gene product of interest. Alternatively, an inhibitory polynucleotide can comprise a nucleotide sequence that encodes an inhibitory nucleotide molecule that is designed to silence expression of the gene product of interest, such as sense-orientation RNA, antisense RNA, double-stranded RNA (dsRNA), hairpin RNA (hpRNA), intron-containing hpRNA, catalytic RNA, miRNA, and the like. In yet other embodiments, the inhibitory polynucleotide can comprise a nucleotide sequence that encodes a mRNA, the translation of which yields a polypeptide that inhibits expression or function of the target gene product of interest. In this manner, where the inhibitory polynucleotide comprises a nucleotide sequence that encodes an inhibitory nucleotide molecule or a mRNA for a polypeptide, the encoding sequence is operably linked to a promoter that drives expression in a plant cell so that the encoded inhibitory nucleotide molecule or mRNA can be expressed.

Inhibitory sequences are designated herein by the name of the target gene product. Thus, for example, a “threonine deaminase (TD) inhibitory sequence” (also referred to as a “threonine dehydratase (TD) inhibitory sequence”) would refer to an inhibitory sequence that is capable of inhibiting the expression of a threonine deaminase (TD), for example, at the level of transcription and/or translation, or which is capable of inhibiting the function of a TD. Similarly, a “glutamine synthetase (GS) inhibitory sequence” would refer to an inhibitory sequence that is capable of inhibiting the expression of a glutamine synthetase (GS), at the level of transcription and/or translation, or which is capable of inhibiting the function of a GS. As noted elsewhere herein, the targeted OS may be a cytosol-localized GS, such as GS1, in which case the inhibitory sequence would be referred to as a “GS1 inhibitory sequence,” or may be a plastid-localized GS, such as GS2, in which case the GS inhibitory sequence would be referred to as a “GS2 inhibitory sequence.” In like manner, a “biotin synthase (BS) inhibitory sequence” would refer to an inhibitory sequence that is capable of inhibiting the expression of a biotin synthase (BS), at the level of transcription and/or translation, or which is capable of inhibiting the function of a BS. When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (i.e., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (i.e., inhibits expression or function of the target gene product).

Thus, the present invention is directed to methods for introducing an auxotrophic requirement for an essential compound into a plant or plant part, particularly a plant or plant part that is transgenic for a trait of interest. The auxotrophic requirement for the essential compound can be introduced by way of mutation, by way of introduction of an inhibitory polynucleotide construct, or by traditional breeding strategies, in which case the auxotrophic trait is bred into a recipient plant of interest. The methods find use in biocontaining transgenic plants or plant parts. Compositions of the invention thus include transgenic plants or plant parts that are auxotrophic for one or more essential compounds, for example an amino acid, fatty acid, carbohydrate, nucleic acid, vitamin, plant hormone, or precursor thereof, or any combination thereof. In some embodiments, the transgenic plants serve as hosts for production of recombinant proteins, particularly recombinant mammalian proteins of pharmaceutical interest.

The methods of the invention target the suppression (i.e., inhibition) of the expression of one or more components of a biosynthetic pathway for an essential compound such as an amino acid, fatty acid, carbohydrate, nucleic acid, vitamin, plant hormone, or precursor thereof. In some embodiments, the methods target suppression of the expression of one or more components of a biosynthetic pathway for an essential amino acid, for example, isoleucine or glutamine. In other embodiments, the methods target suppression of the expression of one or more components of a biosynthetic pathway for an essential vitamin, for example, biotin. Although the following discussion is directed to the introduction of an auxotrophic requirement for isoleucine, glutamine, or biotin, it is recognized that the methods described here below are applicable to any component of a biosynthetic pathway for an amino acid, fatty acid, carbohydrate, nucleic acid, vitamin, plant hormone, or precursor thereof, particularly when equipped with the methods, compositions, and guidance provided herein.

Thus, in some embodiments, the methods for introducing an auxotrophic requirement into a transgenic plant or plant part target the suppression of a component of the biosynthetic pathway for isoleucine, glutamine, or biotin. Of particular interest is suppression of a threonine deaminase (TD), glutamine synthetase (GS), or biotin synthase (BS), or one or more isoforms thereof. It is recognized that suppression of the TD, GS, or BS and one or more isoforms thereof can be accomplished transiently. Alternatively, by stably suppressing the expression of the TD, GS, or BS protein, it is possible to produce auxotrophic transgenic plants that carry over from generation to generation, either asexually or sexually, the auxotrophic requirement.

Inhibition of the expression of one or more components of a biosynthetic pathway for an essential compound in a plant, for example, a dicotyledonous or monocotyledonous plant, for example, a duckweed plant, can be carried out using any suppression method known in the art. In this manner, a polynucleotide comprising an inhibitory sequence for a component of a biosynthetic pathway for an essential compound, such as an amino acid, fatty acid, carbohydrate, nucleic acid, vitamin, plant hormone, or precursor thereof, is introduced into the plant cell of interest. For transient suppression, the inhibitory sequence can be a chemically synthesized or in vitro-produced small interfering RNA (siRNA) or micro RNA (miRNA) that, when introduced into the plant cell, would directly, though transiently, inhibit the component of the biosynthetic pathway for the essential compound by silencing expression of the targeted gene product (i.e., the pathway component). Thus, for example, where auxotrophy for an essential amino acid is the objective, the inhibitory polynucleotide is designed to inhibit expression of one or more components of a biosynthetic pathway for that amino acid. For example, where the auxotrophic requirement is for isoleucine, the inhibitory polynucleotide is designed to inhibit expression of one or more components of the biosynthetic pathway for this amino acid, for example, by targeting TD, AHS, AHR, DAD, or VIAT, as noted herein above. Where the auxotrophic requirement is for glutamine, the inhibitory polynucleotide is designed to inhibit expression of one or more components of the biosynthetic pathway for this amino acid, for example, by targeting GS1 and/or GS2.

In like manner, where auxotophy for an essential vitamin is the objective, the inhibitory polynucleotide is designed to inhibit expression of one or more components of a biosynthetic pathway for this vitamin. For example, where the vitamin is biotin, the inhibitory polynucleotide is designed to inhibit expression of one or more components of the biosynthetic pathway for this vitamin, for example, by targeting KAPA, DAPA, DBS, or BS.

Alternatively, stable suppression of the expression of one or more components of a biosynthetic pathway for an essential compound advantageously introduces an auxotrophic requirement that is heritable from generation to generation. Thus, in some embodiments, the activity of a component of a biosynthetic pathway for the essential compound, such as an amino acid, fatty acid, carbohydrate, nucleic acid, vitamin, plant hormone, or precursor thereof, is reduced or eliminated by transforming a plant cell with an expression cassette that expresses a polynucleotide that inhibits the expression of the component of the biosynthetic pathway for that essential compound. The polynucleotide may inhibit the expression of the component of the biosynthetic pathway directly, by preventing transcription or translation of the pathway-component messenger RNA, or indirectly, by encoding a polypeptide that inhibits the transcription or translation of a gene encoding the pathway component. Methods for inhibiting or eliminating the expression of a gene in a plant are well known in the art, and any such method may be used in the present invention to inhibit the expression of at least one component of a biosynthetic pathway for the essential compound for which the plant is to have an auxotrophic requirement.

Thus, in some embodiments, expression of a component of a biosynthetic pathway for an essential amino acid, carbohydrate, nucleic acid, fatty acid, vitamin, or plant hormone can be inhibited by introducing into the plant a nucleotide construct, such as an expression cassette, comprising a sequence that encodes an inhibitory nucleotide molecule that is designed to silence expression of the gene product of interest (for example, TD, GS1, GS2, or BD, as exemplified herein), such as sense-orientation RNA, antisense RNA, double-stranded RNA (dsRNA), hairpin RNA (hpRNA), intron-containing hpRNA, catalytic RNA, miRNA, and the like. In other embodiments, the nucleotide construct, for example, an expression cassette, can comprise a sequence that encodes a mRNA, the translation of which yields a polypeptide of interest that inhibits expression or function of the gene product of interest (for example, TD, GS1, GS2, or BD, as exemplified herein). Where the nucleotide construct comprises a sequence that encodes an inhibitory nucleotide molecule or a mRNA for a polypeptide of interest, the sequence is operably linked to a promoter that drives expression in a plant cell so that the encoded inhibitory nucleotide molecule or mRNA can be expressed.

In accordance with the present invention, the expression of a gene encoding a component of a biosynthetic pathway for an essential compound (for example, an amino acid, fatty acid, carbohydrate, nucleic acid, vitamin, plant hormone, or precursor thereof) is inhibited if the protein level of the gene product of interest (for example, TD, GS1, GS2, or BD, as exemplified herein) is statistically lower than the protein level of the same gene product in a plant that has not been genetically modified or mutagenized to inhibit the expression of that gene product. In particular embodiments of the invention, the protein level of the pathway component (for example, TD, GS1, GS2, or BD, as exemplified herein) in a modified plant according to the invention is less than 95%, less than 90%, less than 80%, less than 70%, less than 60%, less than 50%, less than 40%, less than 30%, less than 20%, less than 10%, or less than 5% of the protein level of the same pathway component (for example, TD, GS1, GS2, or BD, as exemplified herein) in a plant that is not a mutant or that has not been genetically modified to inhibit the expression of that pathway component. The expression level of the pathway component of interest (for example, TD, GS1, GS2, or BD, as exemplified herein), may be measured directly, for example, by assaying for the level of that pathway component expressed in the plant cell or plant, or indirectly, for example, by observing the effect in a transgenic plant at the phenotypic level, i.e., by transgenic plant analysis, observed as an auxotrophic requirement for the essential compound, the biosynthesis of which has been reduced or eliminated in the plant as a result of the inhibition of expression of the targeted pathway component.

In other embodiments of the invention, the activity of a component of a biosynthetic pathway for an essential compound, such as an amino acid, fatty acid, carbohydrate, nucleic acid, vitamin, plant hormone, or precursor thereof, is reduced or eliminated by transforming a plant cell with an expression cassette comprising a polynucleotide encoding a polypeptide that inhibits the activity of that pathway component (for example, TD, GS1, GS2, or BD, as exemplified herein). The activity of a biosynthetic pathway component is inhibited according to the present invention if the activity of the pathway component (for example, TD, GS1, GS2, or BD, as exemplified herein) is statistically lower than the activity of the same pathway component in a plant that has not been genetically modified to inhibit the activity of that pathway component. In particular embodiments of the invention, the activity of the pathway component (for example, TD, GS1, GS2, or BD, as exemplified herein) in a modified plant according to the invention is less than 95%, less than 90%, less than 80%, less than 70%, less than 60%, less than 50%, less than 40%, less than 30%, less than 20%, less than 10%, or less than 5% of the activity of the same pathway component in a plant that has not been genetically modified to inhibit the expression of that pathway component. The activity of a pathway component (for example, TD, GS1, GS2, or BD, as exemplified herein) is “eliminated” according to the invention when it is not detectable by suitable assay methods known to those of skill in the art, including those assays described elsewhere herein.

In other embodiments, the activity of a component of a biosynthetic pathway for an essential compound may be reduced or eliminated by disrupting the gene encoding the pathway component. The invention encompasses mutagenized plants, particularly plants that are components of the duckweed family, that carry mutations in a gene encoding a component of a biosynthetic pathway for an essential compound (for example, in a gene encoding TD, GS1, GS2, or BD, as exemplified here) where the mutations reduce expression of the gene encoding the pathway component or inhibit the activity of the encoded pathway component.

The methods of the invention can involve any method or mechanism known in the art for reducing or eliminating the activity or level of a component of a biosynthetic pathway for an essential compound, such as an amino acid, fatty acid, carbohydrate, nucleic acid, vitamin, plant hormone, or precursor thereof, in the cells of a plant, including, but not limited to, antisense suppression, sense suppression, RNA interference, directed deletion or mutation, dominant-negative strategies, and the like. Thus, the methods and compositions disclosed herein are not limited to any mechanism or theory of action and include any method where expression or function of a biosynthetic pathway component for an essential compound (for example, TD, GS1, GS2, or BD, as exemplified herein) is inhibited in the cells of the plant of interest, whereby the plant has an auxotrophic requirement for that essential compound.

For example, in some embodiments, the inhibitory sequence for the biosynthetic pathway component is expressed in the sense orientation, wherein the sense-oriented transcripts cause cosuppression of the expression of the pathway component. Alternatively, the inhibitory sequence (e.g., the full-length sequence for the gene encoding the pathway component of interest, or truncated sequence, fragments of the sequence, combinations thereof, and the like) can be expressed in the antisense orientation and thus inhibit endogenous expression of the biosynthetic pathway component by antisense mechanisms.

In yet other embodiments, the inhibitory sequence or sequences that target expression of a biosynthetic pathway component are expressed as a hairpin RNA, which comprises both a sense sequence and an antisense sequence. In embodiments comprising a hairpin structure, the loop structure may comprise any suitable nucleotide sequence including for example 5′ untranslated and/or translated regions of the gene to be suppressed. Thus, for example, where the gene to be suppressed is a TD, GS1, GS2, or BS gene, the loop portion of the hairpin structure may respectively comprise the 5′ UTR and/or translated region of the TD polynucleotide of SEQ ID NO: 1, 2, 4, or 5, the 5′ UTR and/or translated region of the GS1 polynucleotide of SEQ ID NO:7, 8, 10, or 11, the 5′ UTR and/or translated region of the GS2 polynucleotide of SEQ ID NO:13, 14, 16, or 17, or the 5′ UTR and/or translated region of the BS polynucleotide of SEQ ID NO:19, 20, 22, or 23, and the like. In some embodiments, the inhibitory sequence for the pathway component that is expressed as a hairpin is encoded by an inverted region of the nucleotide sequence for the target gene that encodes that pathway component. In yet other embodiments, the inhibitory sequence for the pathway component is expressed as double-stranded RNA, where one inhibitory sequence for the pathway component is expressed in the sense orientation and another complementary sequence for the pathway component is expressed in the antisense orientation. Double-stranded RNA, hairpin structures, and combinations thereof comprising nucleotide sequences from the gene encoding the pathway component (for example, sequences from the TD, GS1, GS2, or BS genes of the invention) may operate by RNA interference, cosuppression, antisense mechanism, any combination thereof, or by means of any other mechanism that causes inhibition of expression or function of that pathway component (for example, the TD, GS1, GS2, or BS polypeptides of the invention).

Thus, many methods may be used to reduce or eliminate the activity of a component of a biosynthetic pathway for an essential compound, and any isoforms thereof. By “isoform” is intended a naturally occurring protein variant of the biosynthetic pathway component of interest, where the variant is encoded by a different gene. Generally, isoforms of a particular protein of interest are encoded by a nucleotide sequence having at least 90% sequence identity to the nucleotide sequence encoding the protein of interest. Thus, for example, the TD protein of SEQ ID NO:3 (L. minor TD isoform #1) and the TD protein of SEQ ID NO:6 (L. minor isoform #2) represent naturally occurring isoforms that are encoded by two genes that share at least 90% sequence identity (compare SEQ ID NO:1 or 2 with SEQ ID NO:4 or 5, respectively; see Table 10). In like manner, the GS1 protein of SEQ ID NO:9 (L. minor GS1 isoform #1) and the GS1 protein of SEQ ID NO:12 (L. minor GS1 isoform #2) represent naturally occurring isoforms that are encoded by two genes that share at least 90% sequence identity (compare SEQ ID NO:7 or 8 with SEQ ID NO:10 or 11, respectively; see Table 10). The GS2 protein of SEQ ID NO:15 (L. minor GS2 isoform #1) and the GS2 protein of SEQ ID NO:18 (L. minor GS2 isoform #2) represent naturally occurring isoforms that are encoded by two genes that share at least 90% sequence identity (compare SEQ ID NO:13 or 14 with SEQ ID NO:16 or 17, respectively; see Table 10). Also, the BS protein of SEQ ID NO:21 (L. minor BS isoform #1) and the BS protein of SEQ ID NO:24 (L. minor BS isoform #2) represent naturally occurring isoforms that are encoded by two genes that share at least 90% sequence identity (compare SEQ ID NO:19 or 20 with SEQ ID NO:22 or 23, respectively; see Table 10).

More than one method may be used to reduce or eliminate the activity of a biosynthetic pathway component, and isoforms thereof. Non-limiting examples of methods of reducing or eliminating the activity of a plant biosynthetic pathway component for an essential compound such as an amino acid, fatty acid, carbohydrate, nucleic acid, vitamin, plant hormone, or precursor thereof, are given below. Although these methods are exemplified for components of biosynthetic pathways for the amino acids isoleucine and glutamine, and the vitamin biotin, it is recognized that the methods are applicable to any component of a biosynthetic pathway for an essential compound, for example, an amino acid, fatty acid, carbohydrate, nucleic acid, vitamin, plant hormone, or precursor thereof, for which an auxotrophic requirement is to be introduced into a transgenic plant or plant part thereof.

### Polynucleotide-Based Methods:

In some embodiments of the present invention, a plant cell is transformed with an expression cassette that is capable of expressing a polynucleotide that inhibits the expression of a component of a biosynthetic pathway for an essential compound of interest, for example, an amino acid, fatty acid, carbohydrate, nucleic acid, vitamin, plant hormone, or precursor thereof. In some embodiments, the essential compound is an amino acid such as isoleucine or glutamine, and the pathway component is TD or GS1 and/or GS2, respectively. In other embodiments, the essential compound of interest is a vitamin such as biotin, and the pathway component is BS. The term “expression” as used herein refers to the biosynthesis of a gene product, including the transcription and/or translation of the gene product. For example, for the purposes of the present invention, an expression cassette capable of expressing a polynucleotide that inhibits the expression of at least one TD, GS1, GS2, or BS is an expression cassette capable of producing an RNA molecule that inhibits the transcription and/or translation of at least one TD, GS1, GS2, or BS. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a biosynthetic pathway component for an essential compound, for example, TD (targeting isoleucine production), GS (targeting cytosol-localized glutamine production), GS2 (targeting plastid-localized glutamine production), or BS (targeting biotin production), are given below.

Sense Suppression/Cosuppression

In some embodiments of the invention, inhibition of the expression of a component of a biosynthetic pathway for an essential compound such as an amino acid, fatty acid, carbohydrate, nucleic acid, vitamin, plant hormone, or precursor thereof, may be obtained by sense suppression or cosuppression. For cosuppression, an expression cassette is designed to express an RNA molecule corresponding to all or part of a messenger RNA encoding a biosynthetic pathway component (for example, an enzyme involved in the biosynthesis of isoleucine, glutamine, or biotin, such as TD, GS1 and/or GS2, or BS, respectively) in the “sense” orientation. Overexpression of the RNA molecule can result in reduced expression of the native gene encoding the pathway component. Accordingly, multiple plant lines transformed with the cosuppression expression cassette are screened to identify those that show the greatest inhibition of expression of the targeted pathway component.

The polynucleotide used for cosuppression may correspond to all or part of the sequence encoding the pathway component, all or part of the 5′ and/or 3′ untranslated region of a transcript for the pathway component, or all or part of both the coding sequence and the untranslated regions of a transcript encoding the pathway component. In some embodiments where the polynucleotide comprises all or part of the coding region for the pathway component, the expression cassette is designed to eliminate the start codon of the polynucleotide so that no protein product will be transcribed.

Cosuppression may be used to inhibit the expression of plant genes to produce plants having undetectable protein levels for the proteins encoded by these genes. See, for example, Broin et al. (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al. (2002) Plant Physiol. 129:1723-1731; Yu et al. (2003) Phytochemistry 63:753-763; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. Patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

Antisense Suppression

In some embodiments of the invention, inhibition of the expression of a component of a biosynthetic pathway for an essential compound such as an amino acid, fatty acid, carbohydrate, nucleic acid, vitamin, plant hormone, or precursor thereof, may be obtained by antisense suppression. For antisense suppression, the expression cassette is designed to express an RNA molecule complementary to all or part of a messenger RNA encoding the pathway component (for example, an enzyme involved in the biosynthesis of isoleucine, glutamine, or biotin, such as TD, GS1 and/or GS2, or BS, respectively). Overexpression of the antisense RNA molecule can result in reduced expression of the native gene encoding the pathway component. Accordingly, multiple plant lines transformed with the antisense suppression expression cassette are screened to identify those that show the greatest inhibition of expression of the targeted pathway component.

The polynucleotide for use in antisense suppression may correspond to all or part of the complement of the sequence encoding the pathway component, all or part of the complement of the 5′ and/or 3′ untranslated region of the transcript for the pathway component, or all or part of the complement of both the coding sequence and the untranslated regions of a transcript encoding the pathway component. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al. (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. Patent Publication No. 20020048814, herein incorporated by reference.

Double-Stranded RNA Interference

In some embodiments of the invention, inhibition of the expression of a component of a biosynthetic pathway for an essential compound such as an amino acid, fatty acid, carbohydrate, nucleic acid, vitamin, plant hormone, or precursor thereof, may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of expression the targeted biosynthetic pathway component. Methods for using dsRNA interference to inhibit the expression of endogenous plant genes are described in Waterhouse et al. (1998) Proc. Natl. Acad. Sci. USA 95:13959-13964, Liu et al. (2002) Plant Physiol. 129:1732-1743, and WO 99/49029, WO 99/53050, WO 99/61631, and WO 00/49035; each of which is herein incorporated by reference.

Hairpin RNA Interference and Intron-Containing Hairpin RNA Interference

In some embodiments of the invention, inhibition of the expression of a component of a biosynthetic pathway for an essential compound of interest, such as an amino acid, fatty acid, carbohydrate, nucleic acid, vitamin, plant hormone, or precursor thereof, may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. Patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in Smith et al. (2000) Nature 407:319-320; Wesley et al. (2001) Plant J. 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. Patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

Transcriptional gene silencing (TGS) may be accomplished through use of hpRNA constructs wherein the inverted repeat of the hairpin shares sequence identity with the promoter region of a gene to be silenced. Processing of the hpRNA into short RNAs that can interact with the homologous promoter region may trigger degradation or methylation to result in silencing (Aufsatz et al. (2002) PNAS 99 (Suppl. 4):16499-16506; Mette et al. (2000) EMBO J. 19(19):5194-5201).

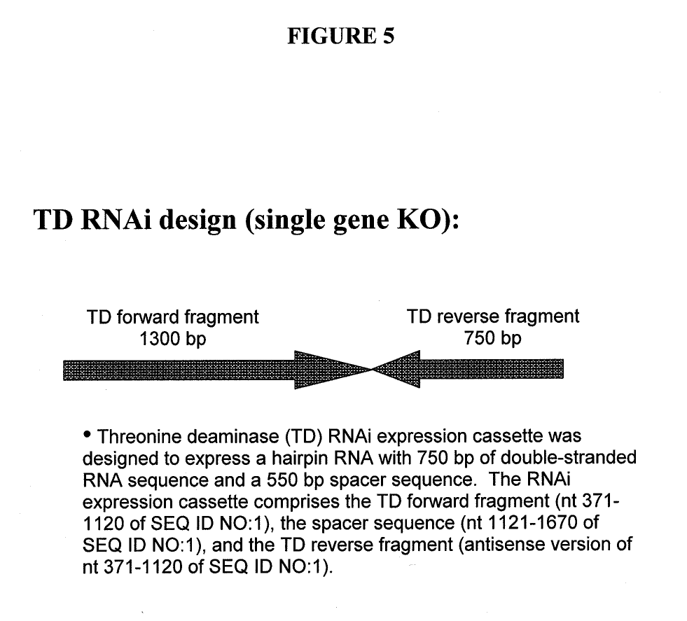

Expression cassettes that are designed to express an RNA molecule that forms a hairpin structure are referred to herein as RNAi expression cassettes. In some embodiments, the RNAi expression cassette is designed in accordance with a strategy outlined in FIG. 5, as exemplified for suppression of expression of TD, and thus introduction of an auxotrophic requirement for isoleucine. See also Example 1 herein below. Where more than one form of the biosynthetic pathway component exists, for example, due to compartmentalization within the plant cell (for example, a cytosolic form and a plastid-localized form, as for GS), an RNAi expression cassette can be designed to suppress the expression of the individual forms of the pathway component (i.e., each cassette provides a single-gene knockout), or can be designed to suppress the expression of both forms of the pathway component (i.e., a single RNAi expression cassette expresses an inhibitory molecule that provides for suppression of expression of both forms of the pathway component, as outlined in FIG. 6, as exemplified for suppression of expression of GS1 and GS2). Where the RNAi expression cassette suppresses expression of both forms of a pathway component, it is referred to herein as a “chimeric” RNAi expression cassette. The single-gene and chimeric RNAi expression cassettes can be designed to express larger hpRNA structures or, alternatively, small hpRNA structures, as noted herein below.

Thus, in some embodiments, the RNAi expression cassette is designed to express larger hpRNA structures having sufficient homology to the target mRNA transcript to provide for post-transcriptional gene silencing of a gene encoding a component of a biosynthetic pathway for an essential compound (for example, an enzyme involved in the biosynthesis of isoleucine, glutamine, or biotin, such as TD, GS1 and/or GS2, or BS). Thus, for example, where the biosynthetic pathway component is a TD, GS1, GS2, or BS, for larger hp RNA structures, the sense strand of the RNAi expression cassette is designed to comprise in the 5′-to-3′ direction the following operably linked elements: a promoter of interest, a forward fragment of the TD, GS1, GS2, or BS gene sequence comprising about 500 to about 800 nucleotides (nt) of a sense strand for TD, GS1, S2, or BS, respectively, a spacer sequence comprising about 100 to about 700 nt of any sequence as noted herein below, and a respective reverse fragment of the TD, GS1, GS2, or BS gene sequence, wherein the reverse fragment comprises the antisense sequence complementary to the respective (i.e., TD, GS1, GS2, or BS) forward fragment. Thus, for example, if a forward fragment is represented by nucleotides “ . . . acttg . . . ”, the corresponding reverse fragment is represented by nucleotides “ . . . caagt . . . ”, and the sense strand for such an RNAi expression cassette would comprise the following sequence: “5′- . . . acttg . . . nnnn . . . caagt . . . -3′, where “nnnn” represents the spacer sequence.

It is recognized that the forward fragment can comprise a nucleotide sequence that is 100% identical to the corresponding portion of the sense strand for the target gene sequence (as exemplified by TD, GS1, GS2, or BS), or in the alternative, can comprise a sequence that shares at least 90%, 91%, 92%, 93%, 94%, or at least 95%, 96%, 97%, or at least 98% or at least 99% sequence identity to the corresponding portion of the sense strand for the target gene (as exemplified by TD, GS1, GS2, or BS) to be silenced. In like manner, it is recognized that the reverse fragment does not have to share 100% sequence identity to the complement of the forward fragment; rather it must be of sufficient length and sufficient complementarity to the forward fragment sequence such that when the inhibitory RNA molecule is expressed, the transcribed regions corresponding to the forward fragment and reverse fragment will hybridize to form the base-paired stem (i.e., double-stranded portion) of the hp RNA structure. By “sufficient length” is intended a length that is at least 10%, at least 15%, at least 20%, at least 30%, at least 40% of the length of the forward fragment, more frequently at least 50%, at least 75%, at least 90%, or least 95% of the length of the forward fragment. By “sufficient complementarity” is intended the sequence of the reverse fragment shares at least 90%, at least 95%, at least 98% sequence identity with the complement of that portion of the forward fragment that will hybridize with the reverse fragment to form the base-paired stem of the hp RNA structure. Thus, in some embodiments, the reverse fragment is the complement (i.e., antisense version) of the forward fragment.

In designing such an RNAi expression cassette, the lengths of the forward fragment, spacer sequence, and reverse fragments are chosen such that the combined length of the polynucleotide that encodes the hpRNA construct is about 650 to about 2500 nt, about 750 to about 2500 nt, about 750 to about 2400 nt, about 1000 to about 2400 nt, about 1200 to about 2300 nt, about 1250 to about 2100 nt, or about 1500 to about 1800. In some embodiments, the combined length of the expressed hairpin construct is about 650 nt, about 700 nt, about 750 nt, about 800 nt, about 850 nt, about 900 nt, about 950 nt, about 1000 nt, about 1050 nt, about 1100 nt, about 1150 nt, about 1200 nt, about 1250 nt, about 1300 nt, about 1350 nt, about 1400 nt, about 1450 nt, about 1500 nt, about 1550 nt, about 1600 nt, about 1650 nt, about 1700 nt, about 1750 nt, about 1800 nt, about 1850 nt, about 1900 nt, about 1950 nt, about 2000 nt, about 2050 nt, about 2100 nt, about 2150 nt, about 2200 nt, about 2250 nt, about 2300 nt, or any such length between about 650 nt to about 2300 nt.

In some embodiments, as exemplified for the target genes encoding a TD, GS1, GS2, or BS, the forward fragment comprises about 500 to about 800 nt, for example, 500, 525, 550, 575, 600, 625, 650, 675, 700, 725, 750, 775, or 800 nt of a sense strand for a TD, GS1, GS2, or BS, for example, of the sense strand set forth in SEQ ID NO:1, 2, 4, or 5 (TD), or SEQ ID NO:7, 8, 10, or 11 (GS1), or SEQ ID NO:13, 14, 16, or 17 (GS2), or SEQ ID NO: 19, 20, 22, or 23 (BS); the spacer sequence comprises about 100 to about 700 nt, for example, 100, 125, 150, 175, 200, 225, 250, 275, 300, 325, 350, 375, 400, 425, 450, 475, 500, 525, 550, 575, 600, 625, 650, 675, or 700 nt of any sequence as noted below, and the reverse fragment comprises the antisense strand for the forward fragment sequence, or a sequence having sufficient length and sufficient complementarity to the forward fragment sequence.

The spacer sequence can be any sequence that has insufficient homology to the target gene, for example, a TD, GS1, GS2, or BS gene, and insufficient homology to itself such that the portion of the expressed inhibitory RNA molecule corresponding to the spacer region fails to self-hybridize, and thus forms the loop of the hairpin RNA structure. In some embodiments, the spacer sequence comprises an intron, and thus the expressed inhibitory RNA molecule forms an ihpRNA as noted herein above. In other embodiments, the spacer sequence comprises a portion of the sense strand for the gene encoding the biosynthetic pathway component, for example, the TD, GS1, GS2, or BS gene to be silenced, for example, a portion of the sense strand set forth in SEQ ID NO:1, 2, 4, or 5 (TD), or SEQ ID NO:7, 8, 10, or 11 (GS1), or SEQ ID NO:13, 14, 16, or 17 (GS2), or SEQ ID NO: 19, 20, 22, or 23 (BS), particularly a portion of the sense strand immediately downstream from the forward fragment sequence (see, for example, the scheme shown in FIG. 5 for a TD RNAi construct).

The operably linked promoter can be any promoter of interest that provides for expression of the operably linked inhibitory polynucleotide within the plant of interest, including one of the promoters disclosed herein below. The regulatory region can comprise additional regulatory elements that enhance expression of the inhibitory polynucleotide, including, but not limited to, the 5′ leader sequences and 5′ leader sequences plus plant introns discussed herein below.

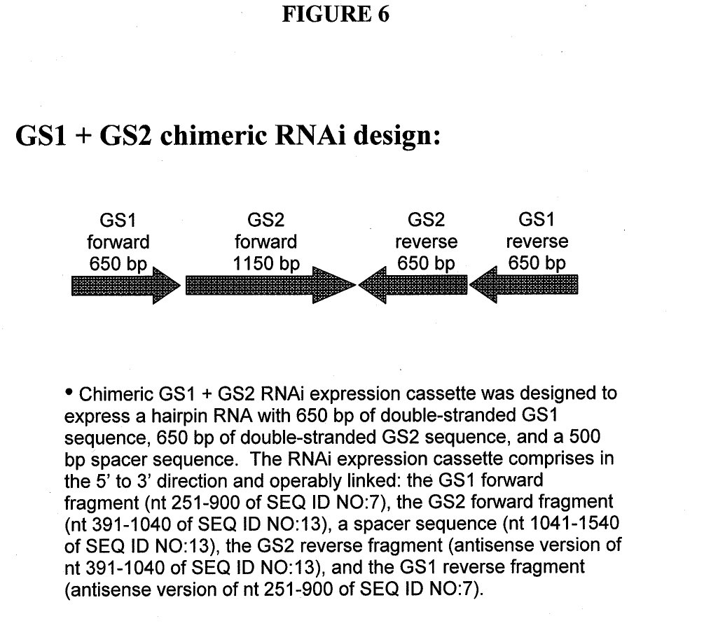

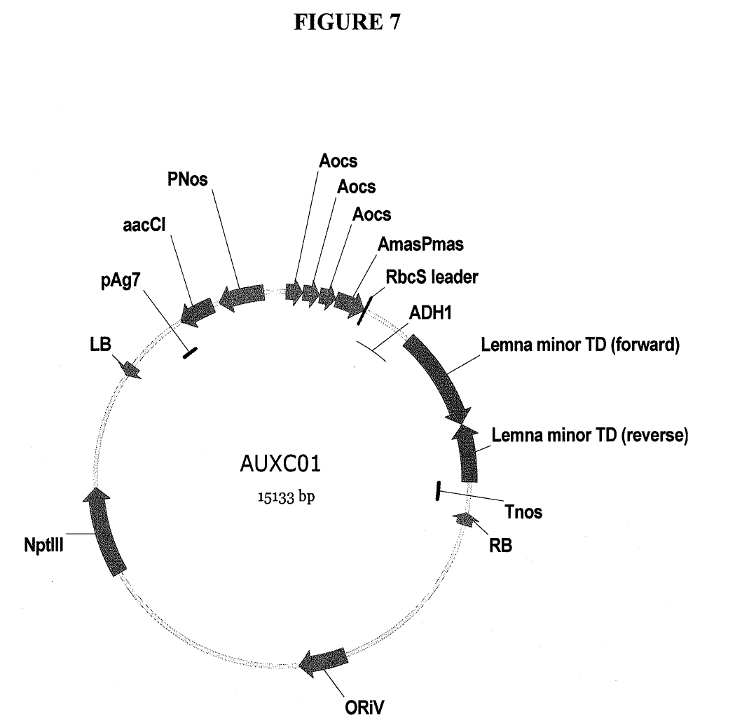

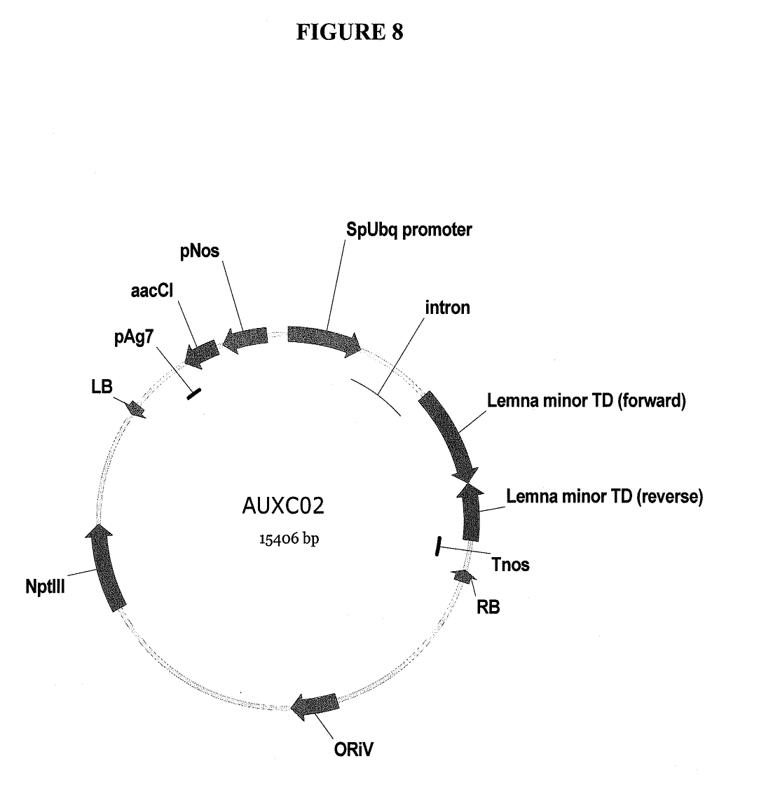

In one embodiment, the RNAi expression cassette is designed to suppress expression of the TD polypeptide of SEQ ID NO:3 or 6, a biologically active variant of the TD polypeptide of SEQ ID NO:3 or 6, or a TD polypeptide encoded by a sequence having at least 75% sequence identity to the sequence of SEQ ID NO:1, 2, 4, or 5, for example, at least 75%, at least 80%, at least 85%, at least 90%, or at least 95% sequence identity to the sequence of SEQ ID NO:1, 2, 4, or 5. In this manner, the sense strand of the RNAi expression cassette is designed to comprise in the 5′-to-3′ direction the following operably linked elements: a promoter of interest; a forward fragment of the TD gene sequence, wherein the forward fragment comprises nt 371-1120 of SEQ ID NO:1; a spacer sequence comprising about 100 to about 700 nt of any sequence as noted above; and a reverse fragment of the TD gene sequence, wherein the reverse fragment comprises the complement (i.e., antisense version) of nt 371-1120 of SEQ ID NO:1. In one such embodiment, the spacer sequence is represented by nt 1121-1670 of SEQ ID NO: 1. Stably transforming a plant with a nucleotide construct comprising this RNAi expression cassette, for example, the vector shown in FIG. 7 or FIG. 8, effectively inhibits expression of TD within the plant cells of the plant in which the hpRNA structure is expressed. In one embodiment, the plant of interest is a component of the duckweed family, for example, a member of the Lemnaceae, and the plant has been stably transformed with the vector shown in FIG. 7 or FIG. 8.

In other embodiments of the invention, the RNAi expression cassette is designed to suppress expression of the GS1 polypeptide of SEQ ID NO:9 or SEQ ID NO:12, a biologically active variant of the GS1 polypeptide of SEQ ID NO:9 or SEQ ID NO:12, or a GS1 polypeptide encoded by a sequence having at least 75% sequence identity to the sequence of SEQ ID NO:7, SEQ ID NO:8, SEQ ID NO:10, or SEQ ID NO: 11, for example, at least 75%, at least 80%, at least 85%, at least 90%, or at least 95% sequence identity to the sequence of SEQ ID NO:7, SEQ ID NO:8, SEQ ID NO:10, or SEQ ID NO:11. In this manner, the sense strand of the RNAi expression cassette is designed to comprise in the 5′-to-3′ direction the following operably linked elements: a promoter of interest; a forward fragment of the GS1 gene sequence, wherein the forward fragment comprises nt 51-700 of SEQ ID NO:7; a spacer sequence comprising about 100 to about 700 nt of any sequence as noted above; and a reverse fragment of the GS1 gene sequence, wherein the reverse fragment comprises the complement (i.e., antisense version) of nt 51-700 of SEQ ID NO:7. In one such embodiment, the spacer sequence is represented by nt 701-1233 of SEQ ID NO:7. Stably transforming a plant with a nucleotide construct comprising this RNAi expression cassette effectively inhibits expression of cytosol-localized GS1 within the plant cells of the plant in which the hpRNA structure is expressed. In one embodiment, the plant of interest is a member of the duckweed family, for example, a member of the Lemnaceae.

In yet other embodiments of the invention, the RNAi expression cassette is designed to suppress expression of the GS2 polypeptide of SEQ ID NO:15 or SEQ ID NO: 18, a biologically active variant of the GS2 polypeptide of SEQ ID NO: 15 or SEQ ID NO:18, or a GS2 polypeptide encoded by a sequence having at least 75% sequence identity to the sequence of SEQ ID NO:13, SEQ ID NO:14, SEQ ID NO:16, or SEQ ID NO: 17, for example, at least 75%, at least 80%, at least 85%, at least 90%, or at least 95% sequence identity to the sequence of SEQ ID NO:13, SEQ ID NO:14, SEQ ID NO:16, or SEQ ID NO:17. In this manner, the sense strand of the RNAi expression cassette is designed to comprise in the 5′-to-3′ direction the following operably linked elements: a promoter of interest; a forward fragment of the GS2 gene sequence, wherein the forward fragment comprises nt 391-1040 of SEQ ID NO:13; a spacer sequence comprising about 100 to about 700 nt of any sequence as noted above; and a reverse fragment of the GS2 gene sequence, wherein the reverse fragment comprises the complement (i.e., antisense version) of nt 391-1040 of SEQ ID NO:13. In one such embodiment, the spacer sequence is represented by nt 1041-1540 of SEQ ID NO:13. Stably transforming a plant with a nucleotide construct comprising this RNAi expression cassette effectively inhibits expression of plastid-localized GS2 within the plant cells of the plant in which the hpRNA structure is expressed. In one embodiment, the plant of interest is a member of the duckweed family, for example, a member of the Lemnaceae.

Where suppression of two forms of a biosynthetic pathway component is desirable, as exemplified herein below for a cytosolic glutamine synthetase (GS1) and a plastid-localized glutamine synthetase (GS2), it can be achieved by introducing single-gene RNAi expression cassettes targeting each form of the component into the plant in a single transformation event, for example, by assembling these single-gene RNAi expression cassettes within a single transformation vector, or as separate co-transformation events, for example, by assembling these single-gene RNAi expression cassettes within two transformation vectors, using any suitable transformation method known in the art, including but not limited to the transformation methods disclosed elsewhere herein.

Where suppression of two forms of a biosynthetic pathway component is desirable, as exemplified herein below for a cytosolic glutamine synthetase (GS1) and a plastid-localized glutamine synthetase (GS2), it can be achieved by introducing single-gene RNAi expression cassettes targeting each form of the component into the plant in a single transformation event, for example, by assembling these single-gene RNAi expression cassettes within a single transformation vector, or as separate co-transformation events, for example, by assembling these single-gene RNAi expression cassettes within two transformation vectors, using any suitable transformation method known in the art, including but not limited to the transformation methods disclosed elsewhere herein.

Alternatively, suppression of both forms of the GS1 and GS2 proteins can be achieved by introducing into the higher plant of interest a chimeric RNAi expression cassette as noted herein above. Thus, in some embodiments of the invention, the sense strand of a chimeric RNAi expression cassette is designed to comprise in the 5′-to-3′ direction the following operably linked elements: a promoter of interest; a chimeric forward fragment, comprising about 500 to about 650 nucleotides (nt) of a sense strand for GS1 and about 500 to about 650 nt of a sense strand for GS2, wherein the GS1 sequence and GS2 sequence can be in either order; a spacer sequence comprising about 100 to about 700 nt of any sequence; and a reverse fragment of the chimeric forward fragment, wherein the reverse fragment comprises the antisense sequence complementary to the respective chimeric forward fragment. See, for example, the scheme shown in FIG. 6.

As previously noted for the individual RNAi expression cassettes, it is recognized that the individual GS1 or GS2 sequence within the chimeric forward fragment can comprise a nucleotide sequence that is 100% identical to the corresponding portion of the sense strand for the target GS1 and GS2 gene sequence, respectively, or in the alternative, can comprise a sequence that shares at least 90%, at least 95%, or at least 98% sequence identity to the corresponding portion of the sense strand for the target GS1 or GS2 gene to be silenced. In like manner, it is recognized that the reverse fragment does not have to share 100% sequence identity to the complement of the chimeric forward fragment; rather it must be of sufficient length and sufficient complementarity to the chimeric forward fragment sequence, as defined herein above, such that when the inhibitory RNA molecule is expressed, the transcribed regions corresponding to the chimeric forward fragment and reverse fragment will hybridize to form the base-paired stem (i.e., double-stranded portion) of the hpRNA structure. In designing such a chimeric RNAi expression cassette, the lengths of the forward fragment, spacer sequence, and reverse fragments are chosen such that the combined length of the polynucleotide that encodes the hpRNA structure is about 1200 to about 3300 nt, about 1250 to about 3300 nt, about 1300 to about 3300 nt, about 1350 to about 3300 nt, about 1400 to about 3300 nt, about 1450 nt to about 3300 nt, about 1500 to about 3300 nt, about 2200 to about 3100 nt, about 2250 to about 2800 nt, or about 2500 to about 2700 nt. In some embodiments, the combined length of the expressed hairpin construct is about 1200 nt, about 1250 nt, about 1300 nt, about 1350 nt, about 1400 nt, about 1450 nt, about 1500 nt, about 1800 nt, about 2200 nt, about 2250 nt, about 2300 nt, about 2350 nt, about 2400 nt, about 2450 nt, about 2500 nt, about 2550 nt, about 2600 nt, about 2650 nt, about 2700 nt, about 2750 nt, about 2800 nt, about 2850 nt, about 2900 nt, about 2950 nt, about 3000 nt, about 3050 nt, about 3100 nt, about 3150 nt, about 3200 nt, about 3250 nt, about 3300 nt, or any such length between about 1200 nt to about 3300 nt.

In some embodiments, the chimeric forward fragment comprises about 500 to about 650 nt, for example, 500, 525, 550, 575, 600, 625, or 650 nt, of a sense strand for GS1, for example, of the sense strand set forth in SEQ ID NO:7, 8, 10, or 11, and about 500 to about 650 nt, for example, 500, 525, 550, 575, 600, 625, or 650 nt, of a sense strand for GS2, for example, of the sense strand set forth in SEQ ID NO:13, 14, 16, or 17, where the GS1 and GS2 sequence can be fused in either order, the spacer sequence comprises about 100 to about 700 nt, for example, 100, 200, 225, 250, 275, 300, 325, 350, 375, 400, 425, 450, 475, 500, 525, 550, 575, 600, 625, 650, 675, or 700 nt of any sequence of interest; and the reverse fragment comprises the antisense strand for the chimeric forward fragment sequence, or a sequence having sufficient length and sufficient complementarity to the chimeric forward fragment sequence.

As noted above for the single-gene RNAi expression cassettes, the spacer sequence can be any sequence that has insufficient homology to the target gene, i.e., GS1 or GS2, and insufficient homology to itself such that the portion of the expressed inhibitory RNA molecule corresponding to the spacer region fails to self-hybridize, and thus forms the loop of the hpRNA structure. In some embodiments, the spacer sequence comprises an intron, and thus the expressed inhibitory RNA molecule forms an ihpRNA as noted herein above. In other embodiments, the spacer sequence comprises a portion of the sense strand for the GS1 or GS2 gene to be silenced, for example, a portion of the sense strand set forth in SEQ ID NO:7, 8, 10, or 11 (GS1) or SEQ ID NO:13, 14, 16, or 17 (GS2). In one embodiment, the chimeric forward fragment comprises the GS1 and GS2 sequence fused in that order, and the spacer sequence comprises a portion of the GS2 sense strand immediately downstream from the GS2 sequence contained within the chimeric forward fragment. In another embodiment, the chimeric forward fragment comprises the GS2 and GS1 sequence fused in that order, and the spacer sequence comprises a portion of the GS1 sense strand immediately downstream from the GS1 sequence contained within the chimeric forward fragment.

In some embodiments, the chimeric RNAi expression cassette is designed to suppress expression of the GS1 polypeptide of SEQ ID NO:9 or SEQ ID NO:12, a biologically active variant of the GS1 polypeptide of SEQ ID NO:9 or SEQ ID NO: 12, or a GS1 polypeptide encoded by a sequence having at least 75% sequence identity to the sequence of SEQ ID NO:7, 8, 10, or 11, for example, at least 75%, at least 80%, at least 85%, at least 90%, or at least 95% sequence identity to the sequence of SEQ ID NO:7, 8, 10, or 11, and to suppress expression of the GS2 polypeptide of SEQ ID NO:15 or SEQ ID NO:18, a biologically active variant of the GS2 polypeptide of SEQ ID NO:15 or SEQ ID NO:18, or a GS2 polypeptide encoded by a sequence having at least 75% sequence identity to the sequence of SEQ ID NO:13, 14, 16, or 17, for example, at least 75%, at least 80%, at least 85%, at least 90%, or at least 95% sequence identity to the sequence of SEQ ID NO:13, 14, 16, or 17. For some of these embodiments, the GS1 sequence within the chimeric forward fragment is chosen such that it corresponds to nt 225 to nt 925 of SEQ ID NO:7, SEQ ID NO:8, SEQ ID NO:10, or SEQ ID NO: 11, and/or the GS2 sequence within the chimeric forward fragment is chosen such that it corresponds to nt 365 to nt 1065 of SEQ ID NO:13, SEQ ID NO:14, SEQ ID NO:16, or SEQ ID NO:17. In other embodiments, the sense strand of the chimeric RNAi expression cassette is designed to comprise in the 5′-to-3′ direction the following operably linked elements: a promoter of interest; a chimeric forward fragment comprising nt 251-900 of SEQ ID NO:7 (GS1 sequence) and nt 391-1040 of SEQ ID NO:13 (GS2 sequence); a spacer sequence comprising about 100 to about 700 nt of any sequence as noted above; and a reverse fragment comprising the complement (i.e., antisense version) of the chimeric forward fragment, i.e., comprising the complement of nt 391-1040 of SEQ ID NO:13 and the complement of nt 251-900 of SEQ ID NO:7. In a particular embodiment, the spacer sequence within this chimeric RNAi expression cassette is represented by nt 1041-1540 of SEQ ID NO:13.

In another such embodiment, the sense strand of the chimeric RNAi expression cassette is designed to comprise in the 5′-to-3′ direction the following operably linked elements: a promoter of interest; a chimeric forward fragment comprising nt 391-1040 of SEQ ID NO:13 (GS2 sequence) and nt 51-700 of SEQ ID NO:7 (GS1 sequence); a spacer sequence comprising about 100 to about 700 nt of any sequence as noted above; and a reverse fragment comprising the complement (i.e., antisense version) of the chimeric forward fragment, i.e., comprising the complement of nt 51-700 of SEQ ID NO:7 and the complement of nt 391-1040 of SEQ ID NO:13. In a particular embodiment, the spacer sequence within this chimeric RNAi expression cassette is represented by nt 701-1233 of SEQ ID NO:7.

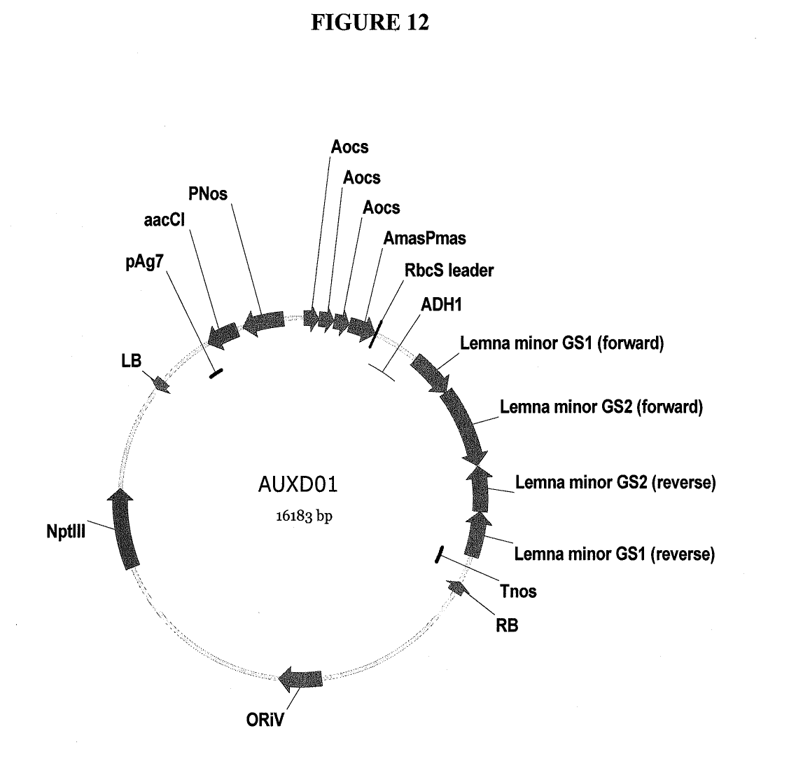

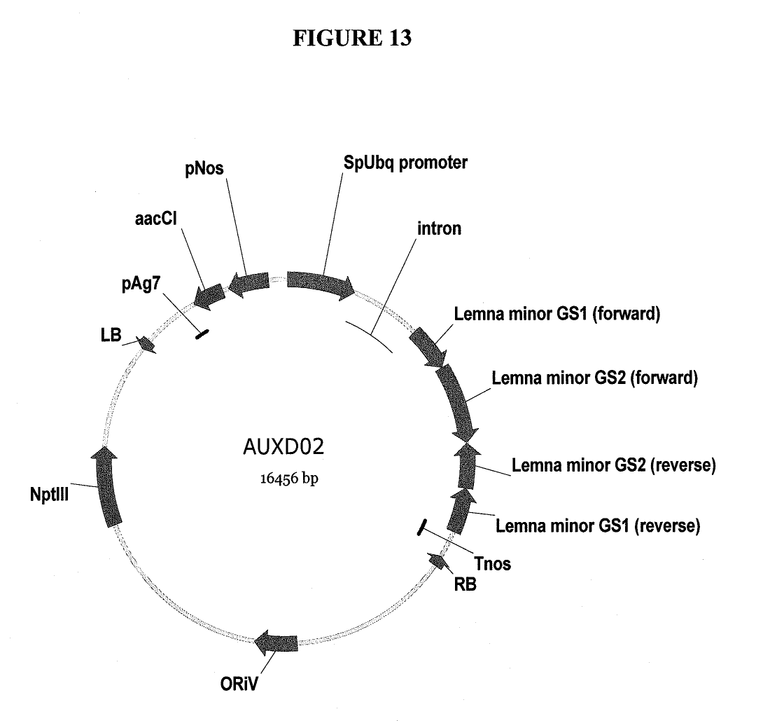

Stably transforming a plant with a nucleotide construct comprising a chimeric RNAi expression cassette described herein, for example, stable transformation with the vector shown in FIG. 12 or FIG. 13, effectively inhibits expression of both GS1 and GS2 within the plant cells of the plant in which the hpRNA structure is expressed. In one embodiment, the plant of interest is a member of the duckweed family, for example, a member of the Lemnaceae, and the plant has been stably transformed with the vector shown in FIG. 12 or FIG. 13.

It is recognized that the plant can be stably transformed with at least two of these chimeric RNAi expression cassettes to provide for very efficient gene silencing of the GS1 and GS2 proteins, including silencing of any isoforms of these two proteins. In this manner, the plant can be stably transformed with a first chimeric RNAi expression cassette wherein the chimeric forward fragment comprises the GS1 and GS2 sequence fused in that order, and the spacer sequence comprises a portion of the GS2 sense strand immediately downstream from the GS2 sequence contained within the chimeric forward fragment; and with a second chimeric RNAi expression cassette wherein the chimeric forward fragment comprises the GS2 and GS1 sequence fused in that order, and the spacer sequence comprises a portion of the GS1 sense strand immediately downstream from the GS1 sequence contained within the chimeric forward fragment.

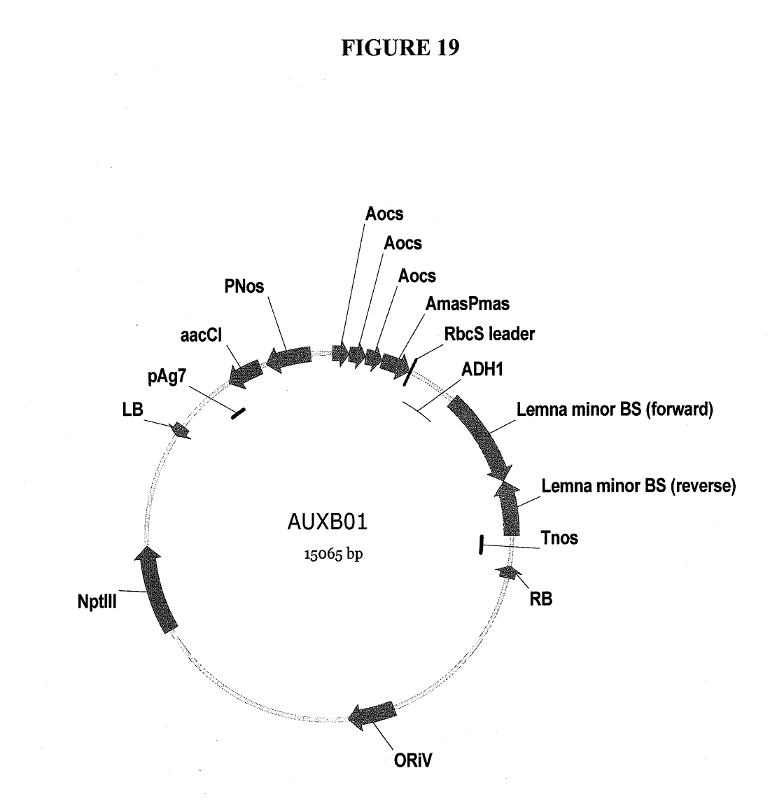

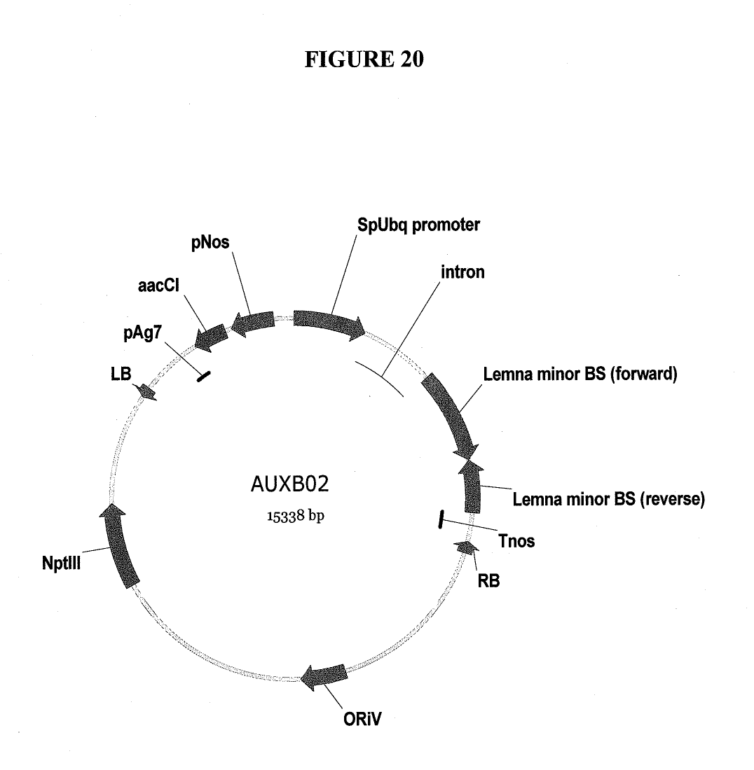

In other embodiments, the sense strand of the RNAi expression cassette is designed to comprise in the 5′-to-3′ direction the following operably linked elements: a promoter of interest; a forward fragment of the BS gene sequence, wherein the forward fragment comprises nt 1-716 of SEQ ID NO:19; a spacer sequence comprising about 100 to about 700 nt of any sequence as noted above; and a reverse fragment of the BS gene sequence, wherein the reverse fragment comprises the complement (i.e., antisense version) of nt 1-716 of SEQ ID NO:19. In one such embodiment, the spacer sequence is represented by nt 717-1266 of SEQ ID NO:19. In another embodiment, the sense strand of the RNAi expression cassette is designed to comprise in the 5′-to-3′ direction the following operably linked elements: a promoter of interest; a forward fragment of the BS gene sequence, wherein the forward fragment comprises nt 1-716 of SEQ ID NO:22; a spacer sequence comprising about 100 to about 700 nt of any sequence as noted above; and a reverse fragment of the BS gene sequence, wherein the reverse fragment comprises the complement (i.e., antisense version) of nt 1-716 of SEQ ID NO:22. In one such embodiment, the spacer sequence is represented by nt 717-1266 of SEQ ID NO:22. Stably transforming a plant with a nucleotide construct comprising such an RNAi expression cassette, for example, the vector shown in FIG. 19 or FIG. 20, effectively inhibits expression of BS within the plant cells of the plant in which the hpRNA structure is expressed. In one embodiment, the plant of interest is a member of the duckweed family, for example, a member of the Lemnaceae, and the plant has been stably transformed with the vector shown in FIG. 19 or FIG. 20.

The operably linked promoter within any of the RNAi expression cassettes encoding large hpRNA structures, or large ihpRNA structures can be any promoter of interest that provides for expression of the operably linked inhibitory polynucleotide within the plant of interest, including one of the promoters disclosed herein below. The regulatory region can comprise additional regulatory elements that enhance expression of the inhibitory polynucleotide, including, but not limited to, the 5′ leader sequences and 5′ leader sequences plus plant introns discussed herein below.

In yet other embodiments, the RNAi expression cassette can be designed to provide for expression of small hpRNA structures having a base-paired stem region comprising about 200 base pairs or less. Expression of the small hpRNA structure is preferably driven by a promoter recognized by DNA-dependent RNA polymerase III. See, for example, U.S. Patent Application No. 20040231016, herein incorporated by reference in its entirety.

In this manner, the RNAi expression cassette is designed such that the transcribed DNA region encodes an RNA molecule comprising a sense and antisense nucleotide region, where the sense nucleotide sequence comprises about 19 contiguous nucleotides having about 90% to about 100% sequence identity to a nucleotide sequence of about 19 contiguous nucleotides from the RNA transcribed from the gene of interest and where the antisense nucleotide sequence comprises about 19 contiguous nucleotides having about 90% to about 100% sequence identity to the complement of a nucleotide sequence of about 19 contiguous nucleotides of the sense sequence. The sense and antisense nucleotide sequences of the RNA molecule should be capable of forming a base-paired (i.e., double-stranded) stem region of RNA of about 19 to about 200 nucleotides, alternatively about 21 to about 90 or 100 nucleotides, or alternatively about 40 to about 50 nucleotides in length. However, the length of the base-paired stem region of the RNA molecule may also be about 30, about 60, about 70 or about 80 nucleotides in length. Where the base-paired stem region of the RNA molecule is larger than 19 nucleotides, there is only a requirement that there is at least one double-stranded region of about 19 nucleotides (wherein there can be about one mismatch between the sense and antisense region) the sense strand of which is “identical” (allowing for one mismatch) with 19 consecutive nucleotides of the target polynucleotide of interest (for example, a TD, GS1, GS2, or BS gene sequence). The transcribed DNA region of this type of RNAi expression cassette may comprise a spacer sequence positioned between the sense and antisense encoding nucleotide region. The spacer sequence is not related to the targeted polynucleotide, and can range in length from 3 to about 100 nucleotides or alternatively from about 6 to about 40 nucleotides. This type of RNAi expression cassette also comprises a terminator sequence recognized by the RNA polymerase III, the sequence being an oligo dT stretch, positioned downstream from the antisense-encoding nucleotide region of the cassette. By “oligo dT stretch” is a stretch of consecutive T-residues. It should comprise at least 4 T-residues, but obviously may contain more T-residues.

It is recognized that in designing the short hpRNA, the fragments of the targeted gene sequence (for example, fragments of a TD, GS1, GS2, or BS gene sequence) and any spacer sequence to be included within the hpRNA-encoding portion of the RNAi expression cassette are chosen to avoid GC-rich sequences, particularly those with three consecutive G/C's, and to avoid the occurrence of four or more consecutive T's or A's, as the string “TTTT . . . ” serves as a terminator sequence recognized by the RNA polymerase III.

Thus, where gene silencing with a short hpRNA is desired, the RNAi expression cassette can be designed to comprise in the 5′-to-3′ direction the following operably linked elements: a promoter recognized by a DNA dependent RNA polymerase III of the plant cell, as defined herein below; a DNA fragment comprising a sense and antisense nucleotide sequence, wherein the sense nucleotide sequence comprises at least 19 contiguous nucleotides having about 90% to about 100% sequence identity to a nucleotide sequence of at least 19 contiguous nucleotides from the sense strand of the gene of interest (for example, a TD, GS1, GS2, or BS gene), and wherein the antisense nucleotide sequence comprises at least 19 contiguous nucleotides having about 90% to about 100% sequence identity to the complement of a nucleotide sequence of at least 19 contiguous nucleotides of the sense sequence, wherein the sense and antisense nucleotide sequence are capable of forming a double-stranded RNA of about 19 to about 200 nucleotides in length; and an oligo dT stretch comprising at least 4 consecutive T-residues.

In some embodiments of the invention, the RNAi expression cassette is designed to express a small hpRNA that suppresses expression of the TD polypeptide of SEQ ID NO:3 or 6, a biologically active variant of the TD polypeptide of SEQ ID NO:3 or 6, or a TD polypeptide encoded by a sequence having at least 90% sequence identity to the sequence of SEQ ID NO:1, SEQ ID NO:2, SEQ ID NO:4, or SEQ ID NO:6. In this manner, the RNAi expression cassette can be designed to comprise in the 5′-to-3′ direction the following operably linked elements: a promoter recognized by a DNA dependent RNA polymerase III of the plant cell, as defined herein below; a DNA fragment comprising a sense and antisense nucleotide sequence, wherein the sense nucleotide sequence comprises at least 19 contiguous nucleotides having about 90% to about 100% sequence identity to a nucleotide sequence of at least 19 contiguous nucleotides of SEQ ID NO:1, 2, 4, or 5, and wherein the antisense nucleotide sequence comprises at least 19 contiguous nucleotides having about 90% to about 100% sequence identity to the complement of a nucleotide sequence of at least 19 contiguous nucleotides of the sense sequence, wherein the sense and antisense nucleotide sequence are capable of forming a double-stranded RNA of about 19 to about 200 nucleotides in length; and an oligo dT stretch comprising at least 4 consecutive T-residues.

In other embodiments of the invention, the RNAi expression cassette is designed to express a small hpRNA that suppresses expression of the GS1 polypeptide of SEQ ID NO:9 or SEQ ID NO:12, a biologically active variant of the GS1 polypeptide of SEQ ID NO:9 or SEQ ID NO:12, or a GS1 polypeptide encoded by a sequence having at least 90% sequence identity to the sequence of SEQ ID NO:7, SEQ ID NO:8, SEQ ID NO:10, or SEQ ID NO:11. In this manner, the RNAi expression cassette can be designed to comprise in the 5′-to-3′ direction the following operably linked elements: a promoter recognized by a DNA dependent RNA polymerase III of the plant cell, as defined herein below; a DNA fragment comprising a sense and antisense nucleotide sequence, wherein the sense nucleotide sequence comprises at least 19 contiguous nucleotides having about 90% to about 100%, sequence identity to a nucleotide sequence of at least 19 contiguous nucleotides of SEQ ID NO:7, 8, 10, or 11, and wherein the antisense nucleotide sequence comprises at least 19 contiguous nucleotides having about 90% to about 100% sequence identity to the complement of a nucleotide sequence of at least 19 contiguous nucleotides of the sense sequence, wherein the sense and antisense nucleotide sequence are capable of forming a double stranded RNA of about 19 to about 200 nucleotides in length; and an oligo dT stretch comprising at least 4 consecutive T-residues.

In yet other embodiments of the invention, the RNAi expression cassette is designed to express a small hpRNA that suppresses expression of the GS2 polypeptide of SEQ ID NO:15 or SEQ ID NO:18, a biologically active variant of the GS2 polypeptide of SEQ ID NO:15 or SEQ ID NO:18, or a GS2 polypeptide encoded by a sequence having at least 90% sequence identity to the sequence of SEQ ID NO:13, SEQ ID NO:14, SEQ ID NO:16, or SEQ ID NO:17. In this manner, the RNAi expression cassette can be designed to comprise in the 5′-to-3′ direction the following operably linked elements: a promoter recognized by a DNA dependent RNA polymerase III of the plant cell, as defined herein below; a DNA fragment comprising a sense and antisense nucleotide sequence, wherein the sense nucleotide sequence comprises at least 19 contiguous nucleotides having about 90% to about 100% sequence identity to a nucleotide sequence of at least 19 contiguous nucleotides of SEQ ID NO:13, 14, 16, or 17, and wherein the antisense nucleotide sequence comprises at least 19 contiguous nucleotides having about 90% to about 100% sequence identity to the complement of a nucleotide sequence of at least 19 contiguous nucleotides of the sense sequence, wherein the sense and antisense nucleotide sequence are capable of forming a double stranded RNA of about 19 to about 200 nucleotides in length; and an oligo dT stretch comprising at least 4 consecutive T-residues.

In still other embodiments of the invention, the RNAi expression cassette is designed to express a small hpRNA that suppresses expression of the BS polypeptide of SEQ ID NO:21 or SEQ ID NO:24, a biologically active variant of the BS polypeptide of SEQ ID NO:21 or SEQ ID NO:24, or a BS polypeptide encoded by a sequence having at least 90% sequence identity to the sequence of SEQ ID NO:19, SEQ ID NO:20, SEQ ID NO:22, or SEQ ID NO:23. In this manner, the RNAi expression cassette can be designed to comprise in the 5′-to-3′ direction the following operably linked elements: a promoter recognized by a DNA dependent RNA polymerase III of the plant cell, as defined herein below; a DNA fragment comprising a sense and antisense nucleotide sequence, wherein the sense nucleotide sequence comprises at least 19 contiguous nucleotides having about 90% to about 100% sequence identity to a nucleotide sequence of at least 19 contiguous nucleotides of SEQ ID NO:19, 20, 22, or 23 and wherein the antisense nucleotide sequence comprises at least 19 contiguous nucleotides having about 90% to about 100% sequence identity to the complement of a nucleotide sequence of at least 19 contiguous nucleotides of the sense sequence, wherein the sense and antisense nucleotide sequence are capable of forming a double stranded RNA of about 19 to about 200 nucleotides in length; and an oligo dT stretch comprising at least 4 consecutive T-residues.

Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the biosynthetic pathway component (for example, an enzyme involved in biosynthesis of isoleucine, glutamine, or biotin such as TD, GS1 and GS2, or BS, respectively). Methods of using amplicons to inhibit the expression of endogenous plant genes are described, for example, in Angell and Baulcombe (1997) EMBO J. 16:3675-3684, Angell and Baulcombe (1999) Plant J. 20:357-362, and U.S. Pat. No. 6,646,805, each of which is herein incorporated by reference.

Ribozymes

In some embodiments, the polynucleotide expressed by the expression cassette of the invention is catalytic RNA or has ribozyme activity specific for the messenger RNA of the biosynthetic pathway component (for example, an enzyme involved in biosynthesis of isoleucine, glutamine, or biotin such as TD, GS1 and GS2, or BS, respectively). Thus, the polynucleotide causes the degradation of the endogenous messenger RNA, resulting in reduced expression of the biosynthetic pathway component. This method is described, for example, in U.S. Pat. No. 4,987,071, herein incorporated by reference.

Small Interfering RNA or Micro RNA

In some embodiments of the invention, inhibition of the expression of a component of a biosynthetic pathway for an essential compound (for example, an enzyme involved in biosynthesis of isoleucine, glutamine, or biotin such as TD, GS1 and GS2, or BS, respectively) may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). Thus, for example, for suppression of TD, GS1, GS2, or BS expression, the 22-nucleotide sequence is selected from a TD, GS1, GS2, or BS transcript sequence, respectively, and contains 22 nucleotides of said TD, GS1, GS2, or BS sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

### Polypeptide-Based Inhibition of Gene Expression

In one embodiment, the polynucleotide encodes a zinc finger protein that binds to a gene encoding a component of a biosynthetic pathway for an essential compound of interest, such as an amino acid, fatty acid, carbohydrate, nucleic acid, vitamin, plant hormone, or precursor thereof (for example, an enzyme involved in biosynthesis of isoleucine, glutamine, or biotin such as TD, GS1 and GS2, or BS, respectively), resulting in reduced expression of the gene. In particular embodiments, the zinc finger protein binds to a regulatory region of a gene encoding the pathway component. In other embodiments, the zinc finger protein binds to a messenger RNA encoding the pathway component and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 20030037355; each of which is herein incorporated by reference.

### Polypeptide-Based Inhibition of Protein Activity

In some embodiments of the invention, the polynucleotide encodes an antibody that binds to a component of a biosynthetic pathway for an essential compound of interest, such as an amino acid, fatty acid, carbohydrate, nucleic acid, vitamin, plant hormone, or precursor thereof, and reduces the activity of the pathway component. In another embodiment, the binding of the antibody results in increased turnover of the antibody-pathway component complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21:35-36, incorporated herein by reference.

In one embodiment, the polynucleotide encodes another type of protein that binds to the pathway component. In one such embodiment, the pathway component is a BS protein, for example, the BS protein set forth in SEQ ID NO:21 or SEQ ID NO:24, and the inhibitory polynucleotide encodes streptavidin, a biotin-binding protein. Overexpression of streptavidin results in inhibition of activity of endogenous biotin as a result of its binding to this endogenous protein. Binding of streptavidin to biotin essentially removes biotin availability for other enzymes that require this cofactor for normal function in plant cells. See Example 3 herein below.

### Gene Disruption

In some embodiments of the present invention, the activity of a component of a biosynthetic pathway for an essential compound, such as an amino acid, fatty acid, carbohydrate, nucleic acid, vitamin, plant hormone, or precursor thereof, is reduced or eliminated by disrupting the gene encoding the pathway component (for example, an enzyme involved in biosynthesis of isoleucine, glutamine, or biotin such as TD, GS1 and GS2, or BS, respectively). The gene encoding the pathway component may be disrupted by any method known in the art. For example, in one embodiment, the gene is disrupted by transposon tagging. In another embodiment, the gene is disrupted by mutagenizing plants using random or targeted mutagenesis, and selecting for plants that have reduced activity for the targeted pathway component.

In one embodiment of the invention, transposon tagging is used to reduce or eliminate the activity of a component of a biosynthetic pathway for an essential compound (for example, an enzyme involved in biosynthesis of isoleucine, glutamine, or biotin such as TD, GS1 and GS2, or BS, respectively). Transposon tagging comprises inserting a transposon within an endogenous gene to reduce or eliminate expression of the encoded gene product. In this embodiment, the expression of the pathway component (for example, an enzyme involved in biosynthesis of isoleucine, glutamine, or biotin such as TD, GS1 and GS2, or BS, respectively) is reduced or eliminated by inserting a transposon within a regulatory region or coding region of the gene encoding the pathway component. A transposon that is within an exon, intron, 5′ or 3′ untranslated sequence, a promoter, or any other regulatory sequence of a gene encoding the pathway component may be used to reduce or eliminate the expression and/or activity of the encoded pathway component.

Methods for the transposon tagging of specific genes in plants are well known in the art. See, for example, Maes et al. (1999) Trends Plant Sd. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928). In addition, the TUSC process for selecting Mu insertions in selected genes has been described in Bensen et al. (1995) Plant Cell 7:75-84; Mena et al. (1996) Science 274:1537-1540; each of which is herein incorporated by reference.

The invention encompasses additional methods for reducing or eliminating the activity of a component of a biosynthetic pathway for an essential compound (for example, an enzyme involved in biosynthesis of isoleucine, glutamine, or biotin such as TD, GS1 and GS2, or BS, respectively). Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham et al. (1999) Proc. Natl. Acad. Sci. USA 96:8774-8778; each of which is herein incorporated by reference.

Additional methods for decreasing, eliminating or interfering with the expression of endogenous genes in plants include other forms of mutagenesis, using mutagenic or carcinogenic compounds including chemical mutagenesis such as ethyl methanesulfonate-induced mutagenesis, UV mutagenesis, deletion mutagenesis and fast neutron deletion mutagenesis used in a reverse genetics sense (with PCR) to identify plant lines in which the endogenous gene has been deleted. For examples of these methods, see, Ohshima et al. (1998) Virology 213:472-481; Okubara et al. (1994) Genetics 137:867-874; and Quesada et al. (2000) Genetics 154:421-436. In addition, a fast and automatable method for screening for chemically induced mutations, Targeting Induced Local Lesions In Genomes (TILLING), using denaturing HPLC or selective endonuclease digestion of selected PCR products can be used herein. See, McCallum et al. (2000) Nat. Biotechnol. 18:455-457.

Mutations that impact gene expression or interfere with the function of the encoded polypeptide can be determined using methods that are well known in the art. Insertional mutations in gene exons usually result in null-mutants. Mutations in conserved residues can be particularly effective in inhibiting the metabolic function of the encoded protein. Conserved residues of plant polypeptides that are components of biosynthetic pathways for essential compounds have been described and are known to those of skill in the art. Dominant mutants can be used to trigger RNA silencing due to gene inversion and recombination of a duplicated gene locus. See, e.g., Kusaba et al. (2003) Plant Cell 15:1455-1467.

Thus inhibition of expression of a component of a biosynthetic pathway for an essential compound, such as an amino acid, fatty acid, carbohydrate, nucleic acid, vitamin, plant hormone, or precursor thereof (for example, an enzyme involved in biosynthesis of isoleucine, glutamine, or biotin such as TD, GS1 and GS2, or BS, respectively) in a plant of interest can be accomplished by any of the foregoing methods in order to introduce an auxotrophic requirement for that essential compound.

Introduction of at least one auxotrophic requirement into a transgenic plant or plant part advantageously provides a method for biocontainment of the transgenic plant or plant part. The present invention also thus includes a method of biocontaining a transgenic plant or plant part having at least one auxotrophic requirement by providing an effective amount of an essential compound to the transgenic plant or plant part so that the plant develops, grows, or survives in the presence of the compound. The transgenic plant or plant part is biocontained by removing the effective amount of the essential compound from the transgenic plant or plant part so that the plant or plant part does not develop, grow, or survive in the absence of the compound.

As used herein, “effective amount” means an amount of an essential compound sufficient to permit development, growth, and survival of a transgenic plant or plant part having an auxotrophic requirement for that essential compound when the effective amount of the essential compound is supplied to the plant from an exogenous source. For example, an effective amount of an amino acid such as isoleucine or glutamine, or a vitamin such as biotin, means that amount of the essential compound that, when supplied to the transgenic plant or plant part that is auxotrophic for that essential compound, allows for the transgenic plant or plant part to develop, grow, and survive.

In one embodiment, the methods of the invention are directed to biocontainment of a transgenic plant or plant part that has an auxotrophic requirement for an amino acid, carbohydrate, fatty acid, nucleic acid, vitamin, plant hormone, precursor thereof or combination thereof. In some of these embodiments, the methods of the invention are directed to biocontainment of a transgenic plant or plant part that is auxotrophic for the amino acid isoleucine or glutamine, or the vitamin biotin, as exemplified herein.

The present invention also includes a method of regulating heterologous polypeptide production in a transgenic plant or plant part having at least one auxotrophic requirement and having a polynucleotide construct encoding a heterologous polypeptide of interest. In this manner, the method comprises providing an effective amount of an essential compound to the transgenic plant or plant part that has an auxotrophic requirement for that compound, so that the plant or plant part develops, grows, and survives, thereby allowing for expression and production of the heterologous polypeptide when all other conditions suitable for expression and production of the polypeptide are met. Production of the heterologous polypeptide is reduced by decreasing the amount of the essential compound provided to the transgenic plant or plant part, and is ceased by removing the effective amount of the essential compound from the transgenic plant or plant part so that the plant fails to develop, grow, or survive, thereby ceasing expression and production of the heterologous polypeptide.

In one such embodiment, the methods of the invention provide for regulation of heterologous polypeptide production in a transgenic plant or plant part that has an auxotrophic requirement for an amino acid, carbohydrate, fatty acid, nucleic acid, vitamin, plant hormone, precursor thereof, or combination thereof. In some of these embodiments, the methods of the invention provide for regulation of heterologous polypeptide production in a transgenic plant or plant part that is auxotrophic for the amino acid isoleucine or glutamine, or the vitamin biotin, as exemplified herein.

For purposes of the present invention, a “polypeptide” refers to any monomeric or multimeric protein or peptide. Methods of the invention that provide for regulation of expression and production of heterologous polypeptides can be applied to any plant host that is transgenic for production of a heterologous polypeptide. Examples of heterologous polypeptides include, but are not limited to, those of interest for use in industrial or chemical processes or as a therapeutic, vaccine, or diagnostics reagent. Exemplary heterologous polypeptides of interest include, but are not limited to, mammalian polypeptides, such as insulin, growth hormone, α-interferon, α-interferon, α-glucocerebrosidase, α-glucoronidase, retinoblastoma protein, p53 protein, angiostatin, leptin, erythropoietin (EPO), granulocyte macrophage colony stimulating factor, plasminogen, tissue plasminogen activator, blood coagulation factors, for example, Factor VII, Factor VIII, Factor IX, and activated protein C, alpha 1-antitrypsin, monoclonal antibodies (mAbs), Fab fragments, single-chain antibodies, cytokines, receptors, hormones, human vaccines, animal vaccines, peptides, and serum albumin.

The methods of the invention can thus be used to regulate heterologous polypeptide expression and production in a transgenic plant or plant part, as well as regulate expression of other polynucleotide constructs of interest (for example, inhibitory polynucleotide constructs that target a gene other than the gene for the component of the biosynthetic pathway for an essential compound for which the plant is to be engineered with an auxotrophic requirement).

### Expression Constructs and Auxotrophic Constructs

The methods of the invention comprise introducing an auxotrophic requirement into a transgenic plant or plant part. As noted above, the auxotrophic requirement can be introduced by mutation, breeding strategies, or by the introduction of a polynucleotide construct comprising an inhibitory nucleotide sequence that targets expression or function of a component of a biosynthetic pathway for an essential compound in the transgenic plant or plant part thereof. Furthermore, the auxotrophic requirement can be introduced into a plant that is already transgenic, or introduced into a plant that will be made transgenic at the time the auxotrophic requirement is introduced, or made transgenic following introduction of the auxotrophic requirement. It is recognized that the transgenic status of the plant may be the result of the introduction of a heterologous polynucleotide of interest (other than the heterologous polynucleotide that confers the auxotrophic requirement) by way of traditional breeding strategies, or by way of any plant transformation technique known to those of skill in the art. The methods of the invention thus contemplate the introduction of expression constructs and/or auxotrophic constructs into plants or plant parts thereof in order to achieve transgenic status and/or auxotrophy, respectively.

As used herein, an “expression construct” means a polynucleotide construct for expressing in a plant or plant part a heterologous polynucleotide that confers a trait of interest to the plant or plant part thereof (other than the auxotrophic requirement). By “trait” is intended the phenotype derived from a particular heterologous polynucleotide or a group of heterologous polynucleotides. The trait of interest can be any desirable trait that alters the phenotype of the plant or plant part thereof. Examples of traits include, but are not limited to, pathogen and disease resistance, herbicide resistance, resistance to environmental stress (for example, drought tolerance, cold tolerance, salt tolerance, and the like), altered carbohydrate, protein, fatty acid/oil, or polymer content and composition, flowering time, sterility, and the like. Other desirable traits include the ability to produce heterologous polypeptides, particularly those for use in industrial or therapeutic applications, for example, mammalian polypeptides, such as those described herein above.

Depending upon the desired trait, the heterologous polynucleotide within an expression construct may comprise a coding sequence for a heterologous polypeptide of interest, for example, a heterologous polypeptide that confers pathogen or disease resistance, herbicide resistance, resistance to environmental stress, altered carbohydrate, protein, fatty acid/oil, or polymer content or composition, or that provides for production of a heterologous polypeptide of interest, for example, a polypeptide for industrial or therapeutic applications. Alternatively, the heterologous polynucleotide within an expression construct may comprise an inhibitory nucleotide sequence that suppresses expression of a target gene of interest (other than a target gene whose expression will be suppressed in order to introduce the auxotrophic requirement into the plant or plant part). The expression construct comprises an expression control element operably linked to the heterologous polynucleotide sequence that confers the trait of interest. Introduction of the expression construct into a plant or plant part of interest, such as a dicot or monocot, for example, a member of the duckweed family, results in the production of transgenic plants or plant parts having the desired trait that is conferred by the heterologous polynucleotide within the expression construct.

For purposes of the present invention, an “auxotrophic construct” means a polynucleotide construct for introducing into a transgenic plant or plant part at least one auxotrophic requirement. The auxotrophic construct may comprise an inhibitory nucleotide sequence that is operably linked to an expression control element for use in expressing an inhibitory RNA transcript that interferes with expression (i.e., transcription and/or translation) of a component within a biosynthetic pathway for the essential compound for which the auxotrophic requirement is to be introduced. In one such embodiment, the auxotrophic construct comprises an RNAi expression cassette, for example, a TD, GS1, GS2, or BD RNAi expression cassette, or a GS1/GS2 chimeric RNAi expression cassette, as described herein above. Alternatively, the auxotrophic construct may comprise an expression control element operably linked to a coding sequence for use in expressing a polypeptide that interferes with expression of a component within a biosynthetic pathway for the essential compound for which the auxotrophic requirement is to be introduced (for example, a zinc finger protein) or which binds to the component, thereby interfering with the activity of that component (for example, an antibody or other protein-binding partner, as exemplified herein for the biotin-binding protein streptavidin).

The expression and auxotrophic constructs can be combined into a single polynucleotide construct under control of the same or separate expression control elements and introduced into a plant or plant part. In other embodiments, the expression and auxotrophic constructs can be separate and under the control of distinct expression control elements and introduced into the plant or plant part singly or together. As such, the plant or plant part can be rendered transgenic prior to being rendered auxotrophic, can be rendered auxotrophic prior to being rendered transgenic or can be rendered transgenic and auxotrophic simultaneously.

Typically, “auxotrophic construct,” “expression cassette,” “expression construct,” “expression vector,” “gene delivery vector,” “gene expression vector,” “gene transfer vector,” “nucleic acid construct,” “polynucleotide construct,” and “vector construct,” all refer to an assembly that is capable of directing the expression of a nucleic acid sequence of interest. Thus, the terms include cloning and expression vehicles.

As used herein, “vector” refers to a DNA molecule such as a plasmid, cosmid, or bacterial phage for introducing a polynucleotide construct, for example, an expression construct or auxotrophic construct, into a plant host cell. Cloning vectors typically contain one or a small number of restriction endonuclease recognition sites at which foreign DNA sequences can be inserted in a determinable fashion without loss of essential biological function of the vector, as well as a marker gene, as described herein below, that is suitable for use in the identification and selection of cells transformed with the cloning vector.

The expression and auxotrophic constructs include one or more expression control elements operably linked to the heterologous polynucleotide of interest. “Operably linked” as used herein in reference to nucleotide sequences refers to multiple nucleotide sequences that are placed in a functional relationship with each other. Generally, operably linked DNA sequences are contiguous and, where necessary to join two protein coding regions, in reading frame.

By “expression control element” is intended a regulatory region of DNA, usually comprising a TATA box, capable of directing RNA polymerase II, or in some embodiments, RNA polymerase m, to initiate RNA synthesis at the appropriate transcription initiation site for a particular coding sequence. An expression control element may additionally comprise other recognition sequences generally positioned upstream or 5′ to the TATA box, which influence (e.g., enhance) the transcription initiation rate. Furthermore, an expression control element may additionally comprise sequences generally positioned downstream or 3′ to the TATA box, which influence (e.g., enhance) the transcription initiation rate.

The transcriptional initiation region (e.g., a promoter) may be native or homologous or foreign or heterologous to the plant host into which the expression construct and/or auxotrophic construct is to be introduced, or could be the natural sequence or a synthetic sequence. By foreign, it is intended that the transcriptional initiation region is not found in the wild-type plant host into which the transcriptional initiation region is introduced. By “functional promoter” is intended the promoter, when operably linked to a sequence encoding a protein of interest, is capable of driving expression (i.e., transcription and translation) of the encoded protein, or, when operably linked to an inhibitory sequence encoding an inhibitory nucleotide molecule (for example, a hairpin RNA, double-stranded RNA, miRNA polynucleotide, and the like), the promoter is capable of initiating transcription of the operably linked inhibitory sequence such that the inhibitory nucleotide molecule is expressed. The promoters can be selected based on the desired outcome. Thus the expression constructs and auxotrophic constructs of the invention can comprise constitutive, tissue-preferred, or other promoters for expression of an operably linked heterologous polynucleotide of interest in plants.

Any suitable promoter known in the art can be employed according to the present invention, including bacterial, yeast, fungal, insect, mammalian, and plant promoters. For example, plant promoters, including duckweed promoters, may be used. Exemplary promoters include, but are not limited to, the Cauliflower Mosaic Virus 35S promoter, the opine synthetase promoters (e.g., nos, mas, ocs, etc.), the ubiquitin promoter, the actin promoter, the ribulose bisphosphate (RubP) carboxylase small subunit promoter, and the alcohol dehydrogenase promoter. The duckweed RubP carboxylase small subunit promoter is known in the art (Silverthorne et al. (1990) Plant Mol. Biol. 15:49). Other promoters from viruses that infect plants, preferably duckweed, are also suitable including, but not limited to, promoters isolated from Dasheen mosaic virus, Chlorella virus (e.g., the Chlorella virus adenine methyltransferase promoter, Mitra et al. (1994) Plant Mol. Biol. 26:85), tomato spotted wilt virus, tobacco rattle virus, tobacco necrosis virus, tobacco ring spot virus, tomato ring spot virus, cucumber mosaic virus, peanut stump virus, alfalfa mosaic virus, sugarcane baciliform badnavirus and the like.

Other suitable expression control elements are disclosed in U.S. Pat. No. 7,622,573. These expression control elements were isolated from ubiquitin genes for several members of the Lemnaceae family, and include a full-length Lemna minor ubiquitin expression control element (SEQ ID NO:1 of that publication, setting forth the promoter plus 5′ UTR (SEQ ID NO:4 of that publication) and intron (SEQ ID NO:7 of that publication)); a full-length Spirodela polyrrhiza ubiquitin expression control element (SEQ ID NO:2 of that publication, setting forth the promoter plus 5′ UTR (SEQ ID NO:5 of that publication) and intron (SEQ ID NO:8 of that publication)); a full-length Lemna aequinoctialis ubiquitin expression control element (SEQ ID NO:3 of that publication, setting forth the promoter plus 5′ UTR (SEQ ID NO:6 of that publication) and intron (SEQ ID NO:9 of that publication)). It is recognized that the individual promoter plus 5′ UTR sequences of these expression control elements, and biologically active variants and fragments thereof, can be used to regulate transcription of operably linked heterologous polynucleotides of interest in plants. Similarly, one or more of the intron sequences set forth in these expression control elements, and biologically active fragments or variants thereof, can be operably linked to a promoter of interest in order to enhance expression of a heterologous polynucleotide of interest that is operably linked to that promoter. See U.S. Pat. No. 7,622,573, herein incorporated by reference in its entirety. In some embodiments, the expression control element utilized in the expression or auxotroph construct is the Spirodela polyrrhiza ubiquitin expression control element set forth in SEQ ID NO:40 of the present application, designated herein as the “full-length SpUbq promoter.”

Expression control elements, including promoters, can be chosen to give a desired level of regulation of expression of a heterologous polynucleotide of interest within an expression construct or auxotrophic construct. For example, in some instances, it may be advantageous to use a promoter that confers constitutive expression (e.g, the mannopine synthase promoter from Agrobacterium tumefaciens). Alternatively, in other situations, for example, where expression of a heterologous polypeptide is concerned, it may be advantageous to use promoters that are activated in response to specific environmental stimuli (e.g., heat shock gene promoters, drought-inducible gene promoters, pathogen-inducible gene promoters, wound-inducible gene promoters, and light/dark-inducible gene promoters) or plant growth regulators (e.g., promoters from genes induced by abscissic acid, auxins, cytokinins, and gibberellic acid). As a further alternative, promoters can be chosen that give tissue-specific expression (e.g., root, leaf; and floral-specific promoters).

The overall strength of a given promoter can be influenced by the combination and spatial organization of cis-acting nucleotide sequences such as upstream activating sequences. For example, activating nucleotide sequences derived from the Agrobacterium tumefaciens octopine synthase gene can enhance transcription from the Agrobacterium tumefaciens mannopine synthase promoter (see U.S. Pat. No. 5,955,646 to Gelvin et al.; also see Lee et al. (2007) Plant Physiol. 145:1294-1300). In the present invention, the expression cassette can contain activating nucleotide sequences inserted upstream of the promoter sequence to enhance the expression of the nucleotide sequence of interest. In one embodiment, the expression construct and/or auxotrophic construct includes three upstream activating sequences derived from the Agrobacterium tumefaciens octopine synthase gene operably linked to a promoter derived from an Agrobacterium tumefaciens mannopine synthase gene (see Lee et al. (2007) Plant Physiol. 145:1294-1300, and U.S. Pat. No. 5,955,646, herein incorporated by reference in their entirety).

The overall strength of a given promoter can also be varied by using fragments or truncated versions of the promoter. By “fragment of an expression control element” is intended a portion of the full-length expression control element. Fragments of an expression control element retain biological activity and hence encompass fragments capable of initiating or enhancing expression of an operably linked polynucleotide of interest. Thus, for example, less than the entire expression control element, for example, the expression control elements described herein, may be utilized to drive expression of an operably linked heterologous polynucleotide of interest within an expression construct and/or auxotrophic construct. The nucleotides of such fragments will usually comprise the TATA recognition sequence of the particular expression control element. Such fragments can be obtained by use of restriction enzymes to cleave the naturally occurring expression control elements disclosed herein; by synthesizing a nucleotide sequence from the naturally occurring sequence of the expression control element DNA sequence; or can be obtained through the use of polymerase chain reaction (PCR) technology. See particularly, Mullis et al. (1987) Methods Enzymol. 155:335-350, and Erlich, ed. (1989) PCR Technology (Stockton Press, New York).

Thus, for example, depending upon the gene targeted for suppression, the strength of the expression control element that is used within an auxotrophic construct will be varied in order to balance the recovery of transgenic plants that have the desired auxotrophic requirement with the ability to maximize growth of those transgenic plants in the presence of an exogenous supply of the essential compound. Thus, for example, where the targeted gene is threonine deaminase (TD), and the expression control element is the full-length SbUbq promoter, it may be desirable to use a truncated version of this promoter, such as the SpUbq117 promoter set forth in SEQ ID NO:41 and described in Example 1 herein below. Given the guidance provided herein, one of skill in the art can readily determine whether a strong constitutive promoter, or a weaker constitutive promoter, is more suited for maximizing suppression of a targeted gene while maximizing recovery of an auxotrophic transgenic plant growth, and optionally maximizing expression of a heterologous polynucleotide within the auxotrophic transgenic plant, in the presence of an exogenous supply of the essential compound.

Where the expression control element will be used to drive expression of an operably linked DNA sequence encoding a small hpRNA molecule, for example, within an RNAi expression cassette described herein above for use in an auxotrophic construct, it is advantageous to use an expression control element comprising a promoter recognized by the DNA dependent RNA polymerase III. As used herein, “a promoter recognized by the DNA dependent RNA polymerase III” is a promoter which directs transcription of the associated DNA region through the polymerase action of RNA polymerase III. These include genes encoding 5S RNA, tRNA, 7SL RNA, U6 snRNA and a few other small stable RNAs, many involved in RNA processing. Most of the promoters used by Pol III require sequence elements downstream of +1, within the transcribed region. A minority of pol III templates however, lack any requirement for intragenic promoter elements. These are referred to as type 3 promoters. By “type 3 Pol III promoters” is intended those promoters that are recognized by RNA polymerase III and contain all cis-acting elements, interacting with the RNA polymerase III upstream of the region normally transcribed by RNA polymerase III. Such type 3 Pol III promoters can be assembled within the RNAi expression cassettes of the invention to drive expression of the operably linked DNA sequence encoding the small hpRNA molecule.

Typically, type 3 Pol III promoters contain a TATA box (located between −25 and −30 in Human U6 snRNA gene) and a Proximal Sequence element (PSE; located between −47 and −66 in Human U6 snRNA). They may also contain a Distal Sequence Element (DSE; located between −214 and −244 in Human U6 snRNA). Type 3 Pol III promoters can be found, e.g., associated with the genes encoding 7SL RNA, U3 snRNA and U6 snRNA. Such sequences have been isolated from Arabidopsis, rice, and tomato. See, for example, SEQ ID NOs:1-8 of U.S. Patent Application Publication No. 20040231016.

Other nucleotide sequences for type 3 Pol III promoters can be found in nucleotide sequence databases under the entries for the A. thaliana gene AT7SL-1 for 7SL RNA (X72228), A. thaliana gene AT7SL-2 for 7SL RNA (X72229), A. thaliana gene AT7SL-3 for 7SL RNA (AJ290403), Humulus lupulus H17SL-1 gene (AJ236706), Humulus lupulus H17SL-2 gene (AJ236704), Humulus lupulus H17SL-3 gene (AJ236705), Humulus lupulus H17SL-4 gene (AJ236703), A. thaliana U6-1 snRNA gene (X52527), A. thaliana U6-26 snRNA gene (X52528), A. thaliana U6-29 snRNA gene (X52529), A. thaliana U6-1 snRNA gene (X52527), Zea mays U3 snRNA gene (Z29641), Solanum tuberosum U6 snRNA gene (Z17301; X60506; S83742), tomato U6 small nuclear RNA gene (X51447), A. thaliana U3C snRNA gene (X52630), A. thaliana U3B snRNA gene (X52629), Oryza sativa U3 snRNA promoter (X79685), tomato U3 small nuclear RNA gene (X14411), Triticum aestivum U3 snRNA gene (X63065), and Triticum aestivum U6 snRNA gene (X63066).

Other type 3 Pol III promoters may be isolated from other varieties of tomato, rice or Arabidopsis, or from other plant species using methods well known in the art. For example, libraries of genomic clones from such plants may be isolated using U6 snRNA, U3 snRNA, or 7SL RNA coding sequences (such as the coding sequences of any of the above mentioned sequences identified by their accession number and additionally the Vicia faba U6snRNA coding sequence (X04788), the maize DNA for U6 snRNA (X52315), or the maize DNA for 7SL RNA (X14661)) as a probe, and the upstream sequences, preferably the about 300 to 400 bp upstream of the transcribed regions may be isolated and used as type 3 Pol III promoters. Alternatively, PCR based techniques such as inverse-PCR or TAIL™-PCR may be used to isolate the genomic sequences including the promoter sequences adjacent to known transcribed regions. Moreover, any of the type 3 Pol III promoter sequences described herein, identified by their accession numbers and SEQ ID NOS, may be used as probes under stringent hybridization conditions or as source of information to generate PCR primers to isolate the corresponding promoter sequences from other varieties or plant species.

Although type 3 Pol III promoters have no requirement for cis-acting elements located with the transcribed region, it is clear that sequences normally located downstream of the transcription initiation site may nevertheless be included in the RNAi expression cassettes of the invention. Further, while type 3 Pol III promoters originally isolated from monocotyledonous plants can effectively be used in RNAi expression cassettes to suppress expression of a target gene in both dicotyledonous and monocotyledonous plant cells and plants, type 3 Pol III promoters originally isolated from dicotyledonous plants reportedly can only be efficiently used in dicotyledonous plant cells and plants, Moreover, the most efficient gene silencing reportedly is obtained when the RNAi expression cassette is designed to comprise a type 3 Pol III promoter derived from the same or closely related species. See, for example, U.S. Patent Application Publication No. 20040231016. Thus, where the plant of interest is a monocotyledonous plant, and small hpRNA interference is the method of choice for inhibiting expression of the gene that is targeted by the auxotrophic construct, the type 3 Pol III promoter preferably is from another monocotyledonous plant.

The expression constructs and auxotrophic constructs of the invention thus include in the 5′-3′ direction of transcription, an expression control element comprising a transcriptional and translational initiation region, a heterologous polynucleotide of interest (for example, a sequence encoding a heterologous protein of interest or a sequence encoding an inhibitory nucleotide sequence that, when expressed, is capable of inhibiting the expression or function of a component of a biosynthetic pathway for an essential compound), and a transcriptional and translational termination region functional in plants. Any suitable termination sequence known in the art may be used in accordance with the present invention. The termination region may be native with the transcriptional initiation region, may be native with the nucleotide sequence of interest, or may be derived from another source. Convenient termination regions are available from the Ti-plasmid of A. tumefaciens, such as the octopine synthetase and nopaline synthetase termination regions. See also Guerineau et al. (1991) Mol. Gen. Genet. 262:141; Proudfoot (1991) Cell 64:671; Sanfacon et al. (1991) Genes Dev. 5:141; Mogen et al. (1990) Plant Cell 2:1261; Munroe et al. (1990) Gene 91:151; Ballas et al. (1989) Nucleic Acids Res. 17:7891; and Joshi et al. (1987) Nucleic Acids Res. 15:9627. Additional exemplary termination sequences are the pea RubP carboxylase small subunit termination sequence and the Cauliflower Mosaic Virus 35S termination sequence. Other suitable termination sequences will be apparent to those skilled in the art, including the oligo dT stretch disclosed herein above for use with type 3 Pol III promoters driving expression of an inhibitory polynucleotide that forms a small hpRNA structure.

Generally, when the expression construct is used apart from the inhibitory sequence (i.e., such as in the case when the plant or plant part is transformed before introduction of an auxotrophic construct), it can include a selectable marker gene for the selection of transformed plants or plant parts. Selectable marker genes include, but are not limited to, genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, De Block et al. (1987) EMBO J. 6:2513-2518; De Block et al. (1989) Plant Physiol. 91:694-701; Fromm et al. (1990) Bio/Technology 8:833-839; Gordon-Kamm et al. (1990) Plant Cell 2:603-618. For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Other selectable marker genes include, but are not limited to, genes encoding neomycin phosphotransferase II (Fraley et al. (1986) CRC Crit. Rev. Plant Sci. 4:1-46); cyanamide hydratase (Maier-Greiner et al. (1991) Proc. Natl. Acad. Sci. USA 88:4260-4264); aspartate kinase; dihydrodipicolinate synthase (Perl et al. (1993) Bio/Technology 11:715-718); bar gene (Toki et al. (1992) Plant Physiol. 100:1503-1507; and Gallo-Meagher and Irvine (1996) Crop Sci. 36:1367-1374); tryptophan decarboxylase (Goddijn et al. (1993) Plant Mol. Biol. 22:907-912); neomycin phosphotransferase (NEO; Southern and Berg (1982) J. Mol. Appl. Gen. 1:327-341); hygromycin phosphotransferase (HPT or HYG; Shimizu et al. (1986) Mol. Cell. Biol. 6:1074-1087); dihydrofolate reductase (DHFR; Kwok et al. (1986) Proc. Natl. Acad. Sci. USA 83:4552-4555); phosphinothricin acetyltransferase (De Block et al. (1987), supra); 2,2-dichloropropionic acid dehalogenase (Buchanan-Wollatron et al. (1989) J. Cell. Biochem. 13D:330); acetohydroxyacid synthase (U.S. Pat. No. 4,761,373); 5-enolpyruvyl-shikimate-phosphate synthase (aroA; Comai et al. (1985) Nature 317:741-744); haloarylnitrilase (Int'l Patent Application Publication No. WO 87/04181); acetyl-coenzyme A carboxylase (Parker et al. (1990) Plant Physiol. 92:1220-1225); dihydropteroate synthase (sulI; Guerineau et al. (1990) Plant Mol. Biol. 15:127-136); and 32 kDa photosystem II polypeptide (psbA; Hirschberg and McIntosh (1983) Science 222:1346-1349).

Also included as selectable marker genes are genes encoding resistance to gentamycin (e.g., eacC1, Wohlleben et al. (1989) Mol. Gen. Genet. 217:202-208); chloramphenicol (Herrera-Estrella et al. (1983) EMBO J. 2:987-995); methotrexate (Herrera-Estrella et al. (1983) Nature 303:209-221; and Meijer et al. (1991) Plant Mol. Biol. 16:807-820); hygromycin (Waldron et al. (1985) Plant Mol. Biol. 5:103-108; Li and Murai (1995) Plant Sci. 108:219-227; and Meijer et al., supra); streptomycin (Jones et al. (1987) Mol. Gen. Genet. 210:86-91); spectinomycin (Bretagne-Sagnard and Chupeau (1996) Transgenic Res. 5:131-137); bleomycin (Hille et al. (1986) Plant Mol. Biol. 7:171-176); sulfonamide (Guerineau et al., supra); bromoxynil (Stalker et al. (1988) Science 242:419-423); 2,4-D (Streber and Willmitzer (1989) Bio/Technology 7:811-816); phosphinothricin (De Block et al. (1987), supra); spectinomycin (Bretagne-Sagnard and Chupeau, supra).

The bar gene confers herbicide resistance to glufosinate-type herbicides, such as phosphinothricin (PPT) or bialaphos and the like. As noted above, other selectable markers that could be used in the vector constructs include, but are not limited to, the pat gene, also for PPT and bialaphos resistance, the ALS gene for imidazolinone resistance, the HPH or HYG gene for hygromycin resistance, the EPSP synthase gene for glyphosate resistance, the Hml gene for resistance to the Hc-toxin, and other selective agents used routinely and known to one of ordinary skill in the art. See, Yarranton (1992) Curr. Opin. Biotech. 3:506-511; Chistopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley and Bourgeois, “Repressor recognition of operator and effectors” 177-220 In: The Operon (Miller and Reznikoff eds., Cold Spring Harbor Laboratory 1980); Brown et al. (1987) Cell 49:603-612; Figge et al. (1988) Cell 52:713-722; Deuschle et al. (1989) Proc. Natl. Acad. Sci. USA 86:5400-5404; Deuschle et al. (1990) Science 248:480-483; Fuerst et al. (1989) Proc. Natl. Acad. Sci. USA 86:2549-2553; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad Sci. USA 88:5072-5076; Wyborski and Short (1991) Nuc. Acids Res. 19:4647-4653; Hillenand-Wissman (1989) Topics in Mol. and Struc. Biol. 10:143-162; Degenkolb et al. (1991) Antimicrob. Agents Chemother. 35:1591-1595; Kleinschnidt et al. (1988) Biochemistry 27:1094-1104; Gatz et al. (1992) Plant J. 2:397-404; Gossen and Bujard (1992) Proc. Natl. Acad. Sci. USA 89:5547-5551; Oliva et al. (1992) Antimicrob. Agents Chemother. 36:913-919; Hlavka et al. (1985) Handb. Exp. Pharmacol. 78:317-392; and Gill and Ptashne (1988) Nature 334:721-724.

The above list of selectable marker genes is not meant to be limiting, as any selectable marker gene can be used in the present invention.

### Modification of Nucleotide Sequences for Enhanced Expression in a Plant Host

Where the plant of interest is also genetically modified to express a heterologous polypeptide of interest, for example, a transgenic plant host serving as an expression system for recombinant production of a heterologous polypeptide, the present invention provides for the modification of the expressed polynucleotide sequence encoding the heterologous protein of interest to enhance its expression in the host plant. Thus, where appropriate, the heterologous polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing nucleotide sequences with plant-preferred codons. See, e.g., U.S. Pat. Nos. 5,380,831 and 5,436,391; Perlak et al. (1991) Proc. Natl. Acad. Sci. USA 15:3324; Iannacome et al. (1997) Plant Mol. Biol. 34:485; and Murray et al., (1989) Nucleic Acids. Res. 17:477, herein incorporated by reference.

In some embodiments of the invention, the transgenic plant into which an auxotrophic requirement is to be introduced is a member of the duckweed family, and the polynucleotide encoding the heterologous polypeptide of interest, for example, a mammalian polypeptide, is modified for enhanced expression of the encoded heterologous polypeptide. In this manner, one such modification is the synthesis of the polynucleotide encoding the heterologous polypeptide of interest using duckweed-preferred codons, where synthesis can be accomplished using any method known to one of skill in the art. The preferred codons may be determined from the codons of highest frequency in the proteins expressed in duckweed. A number of duckweed coding sequences are known to those of skill in the art; see for example, the sequences contained in the GenBank® database, which may be accessed through the website for the National Center for Biotechnology Information, a division of the National Library of Medicine, which is located in Bethesda, Md. Tables showing the frequency of codon usage based on the sequences contained in the most recent GenBank® release may be found on the website for the Kazusa DNA Research Institute in Chiba, Japan. This database is described in Nakamura et al. (2000) Nucleic Acids Res. 28:292.

It is recognized that heterologous genes that have been optimized for expression in duckweed and other monocots, as well as other dicots, can be used in the methods of the invention. See, e.g., EP 0 359 472, EP 0 385 962, WO 91/16432; Perlak et al. (1991) Proc. Natl. Acad. Sci. USA 88:3324; Iannacome et al. (1997) Plant Mol. Biol. 34:485; and Murray et al. (1989) Nuc. Acids Res. 17:477, and the like, herein incorporated by reference. It is further recognized that all or any part of the polynucleotide encoding the heterologous polypeptide of interest may be optimized or synthetic. In other words, fully optimized or partially optimized sequences may also be used. For example, 40%, 45%, 50%, 55%, 60%, 65%, 70%, 75%, 80%, 85%, 87%, 90%, 91%, 92%, 93%, 94%, 95%, 96%, 97%, 98%, 99%, or 100% of the codons may be plant-preferred codons, for example, duckweed-preferred codons. As used herein, “duckweed-preferred codon” means a codon having a frequency of codon usage in duckweed of greater than 17%. Likewise, “Lemna-preferred codon” means a codon having a frequency of codon usage in the genus Lemna of greater than 17%. In one embodiment, between 90 and 96% of the codons are duckweed-preferred codons. The coding sequence of a polynucleotide sequence encoding a heterologous polypeptide of interest may comprise codons used with a frequency of at least 17% in Lemna gibba or Lemna minor. Codon usage in Lemna gibba (Table 1) and Lemna minor (Table 2) is shown below. In some embodiments, Table 1 or Table 2 is used to select duckweed-preferred codons.

TABLE 1
Lemna gibba codon usage from
GenBank® Release 139*

| Amino | | | | |
|---|---|---|---|---|
| Acid | Codon | Number | /1000 | Fraction |
| Gly | GGG | 57.00 | 28.89 | 0.35 |
| Gly | GGA | 8.00 | 4.05 | 0.05 |
| Gly | GGT | 3.00 | 1.52 | 0.02 |
| Gly | GGC | 93.00 | 47.14 | 0.58 |
| Glu | GAG | 123.00 | 62.34 | 0.95 |
| Glu | GAA | 6.00 | 3.04 | 0.05 |
| Asp | GAT | 6.00 | 3.04 | 0.08 |
| Asp | GAC | 72.00 | 36.49 | 0.92 |
| Val | GTG | 62.00 | 31.42 | 0.47 |
| Val | GTA | 0.00 | 0.00 | 0.00 |
| Val | GTT | 18.00 | 9.12 | 0.14 |
| Val | GTC | 51.00 | 25.85 | 0.39 |
| Ala | GCG | 44.00 | 22.30 | 0.21 |
| Ala | GCA | 14.00 | 7.10 | 0.07 |
| Ala | GCT | 14.00 | 7.10 | 0.07 |
| Ala | GCC | 139.00 | 70.45 | 0.66 |
| Arg | AGG | 16.00 | 8.11 | 0.15 |
| Arg | AGA | 11.00 | 5.58 | 0.10 |
| Ser | AGT | 1.00 | 0.51 | 0.01 |
| Ser | AGC | 44.00 | 22.30 | 0.31 |
| Lys | AAG | 116.00 | 58.79 | 1.00 |
| Lys | AAA | 0.00 | 0.00 | 0.00 |
| Asn | AAT | 2.00 | 1.01 | 0.03 |
| Asn | AAC | 70.00 | 35.48 | 0.97 |
| Met | ATG | 67.00 | 33.96 | 1.00 |
| Ile | ATA | 4.00 | 2.03 | 0.06 |
| Ile | ATT | 0.00 | 0.00 | 0.00 |
| Ile | ATC | 63.00 | 31.93 | 0.94 |
| Thr | ACG | 19.00 | 9.63 | 0.25 |
| Thr | ACA | 1.00 | 0.51 | 0.01 |
| Thr | ACT | 6.00 | 3.04 | 0.08 |
| Thr | ACC | 50.00 | 25.34 | 0.66 |
| Trp | TGG | 45.00 | 22.81 | 1.00 |
| End | TGA | 4.00 | 2.03 | 0.36 |
| Cys | TGT | 0.00 | 0.00 | 0.00 |
| Cys | TGC | 34.00 | 17.23 | 1.00 |
| End | TAG | 0.00 | 0.00 | 0.00 |
| End | TAA | 7.00 | 3.55 | 0.64 |
| Tyr | TAT | 4.00 | 2.03 | 0.05 |
| Tyr | TAC | 76.00 | 38.52 | 0.95 |
| Leu | TTG | 5.00 | 2.53 | 0.04 |
| Leu | TTA | 0.00 | 0.00 | 0.00 |
| Phe | TTT | 4.00 | 2.03 | 0.04 |
| Phe | TTC | 92.00 | 46.63 | 0.96 |
| Ser | TCG | 34.00 | 17.23 | 0.24 |
| Ser | TCA | 2.00 | 1.01 | 0.01 |
| Ser | TCT | 1.00 | 0.51 | 0.01 |
| Ser | TCC | 59.00 | 29.90 | 0.42 |
| Arg | CGG | 23.00 | 11.66 | 0.22 |
| Arg | CGA | 3.00 | 1.52 | 0.03 |
| Arg | CGT | 2.00 | 1.01 | 0.02 |
| Arg | CGC | 50.00 | 25.34 | 0.48 |
| Gln | CAG | 59.00 | 29.90 | 0.86 |
| Gln | CAA | 10.00 | 5.07 | 0.14 |
| His | CAT | 5.00 | 2.53 | 0.26 |
| His | CAC | 14.00 | 7.10 | 0.74 |
| Leu | CTG | 43.00 | 21.79 | 0.35 |
| Leu | CTA | 2.00 | 1.01 | 0.02 |
| Leu | CTT | 1.00 | 0.51 | 0.01 |
| Leu | CTC | 71.00 | 35.99 | 0.58 |
| Pro | CCG | 44.00 | 22.30 | 0.31 |
| Pro | CCA | 6.00 | 3.04 | 0.04 |
| Pro | CCT | 13.00 | 6.59 | 0.09 |
| Pro | CCC | 80.00 | 40.55 | 0.56 |

TABLE 2
Lemna minor codon usage from
GenBank® Release 139*

| Amino | | | | |
|---|---|---|---|---|
| Acid | Codon | Number | /1000 | Fraction |
| Gly | GGG | 8.00 | 17.39 | 0.22 |
| Gly | GGA | 11.00 | 23.91 | 0.31 |
| Gly | GGT | 1.00 | 2.17 | 0.03 |
| Gly | GGC | 16.00 | 34.78 | 0.44 |
| Glu | GAG | 25.00 | 54.35 | 0.78 |
| Glu | GAA | 7.00 | 15.22 | 0.22 |
| Asp | GAT | 8.00 | 17.39 | 0.33 |
| Asp | GAC | 16.00 | 34.78 | 0.67 |
| Val | GTG | 21.00 | 45.65 | 0.53 |
| Val | GTA | 3.00 | 6.52 | 0.07 |
| Val | GTT | 6.00 | 13.04 | 0.15 |
| Val | GTC | 10.00 | 21.74 | 0.25 |
| Ala | GCG | 13.00 | 28.26 | 0.32 |
| Ala | GCA | 8.00 | 17.39 | 0.20 |
| Ala | GCT | 6.00 | 13.04 | 0.15 |
| Ala | GCC | 14.00 | 30.43 | 0.34 |
| Arg | AGG | 9.00 | 19.57 | 0.24 |
| Arg | AGA | 11.00 | 23.91 | 0.30 |
| Ser | AGT | 2.00 | 4.35 | 0.05 |
| Ser | AGC | 11.00 | 23.91 | 0.26 |
| Lys | AAG | 13.00 | 28.26 | 0.68 |
| Lys | AAA | 6.00 | 13.04 | 0.32 |
| Asn | AAT | 0.00 | 0.00 | 0.00 |
| Asn | AAC | 12.00 | 26.09 | 1.00 |
| Met | ATG | 9.00 | 19.57 | 1.00 |
| Ile | ATA | 1.00 | 2.17 | 0.08 |
| Ile | ATT | 2.00 | 4.35 | 0.15 |
| Ile | ATC | 10.00 | 21.74 | 0.77 |
| Thr | ACG | 5.00 | 10.87 | 0.28 |
| Thr | ACA | 2.00 | 4.35 | 0.11 |
| Thr | ACT | 2.00 | 4.35 | 0.11 |
| Thr | ACC | 9.00 | 19.57 | 0.50 |
| Trp | TGG | 8.00 | 17.39 | 1.00 |
| End | TGA | 1.00 | 2.17 | 1.00 |
| Cys | TGT | 1.00 | 2.17 | 0.12 |
| Cys | TGC | 7.00 | 15.22 | 0.88 |
| End | TAG | 0.00 | 0.00 | 0.00 |
| End | TAA | 0.00 | 0.00 | 0.00 |
| Tyr | TAT | 1.00 | 2.17 | 0.12 |
| Tyr | TAC | 7.00 | 15.22 | 0.88 |
| Leu | TTG | 3.00 | 6.52 | 0.08 |
| Leu | TTA | 1.00 | 2.17 | 0.03 |
| Phe | TTT | 6.00 | 13.04 | 0.25 |
| Phe | TTC | 18.00 | 39.13 | 0.75 |
| Ser | TCG | 11.00 | 23.91 | 0.26 |
| Ser | TCA | 4.00 | 8.70 | 0.09 |
| Ser | TCT | 6.00 | 13.04 | 0.14 |
| Ser | TCC | 9.00 | 19.57 | 0.21 |
| Arg | CGG | 4.00 | 8.70 | 0.11 |
| Arg | CGA | 4.00 | 8.70 | 0.11 |
| Arg | CGT | 0.00 | 0.00 | 0.00 |
| Arg | CGC | 9.00 | 19.57 | 0.24 |
| Gln | CAG | 11.00 | 23.91 | 0.73 |
| Gln | CAA | 4.00 | 8.70 | 0.27 |
| His | CAT | 0.00 | 0.00 | 0.00 |
| His | CAC | 6.00 | 13.04 | 1.00 |
| Leu | CTG | 9.00 | 19.57 | 0.24 |
| Leu | CTA | 4.00 | 8.70 | 0.11 |
| Leu | CTT | 4.00 | 8.70 | 0.11 |
| Leu | CTC | 17.00 | 36.96 | 0.45 |
| Pro | CCG | 8.00 | 17.39 | 0.29 |
| Pro | CCA | 7.00 | 15.22 | 0.25 |
| Pro | CCT | 5.00 | 10.87 | 0.18 |
| Pro | CCC | 8.00 | 17.39 | 0.29 |

Other modifications can also be made to the polynucleotide encoding the heterologous polypeptide of interest to enhance its expression in a plant host of interest, including duckweed. These modifications include, but are not limited to, elimination of sequences encoding spurious polyadenylation signals, exon-intron splice site signals, transposon-like repeats, and other such well characterized sequences which may be deleterious to gene expression. The G-C content of the sequence may be adjusted to levels average for a given cellular host, as calculated by reference to known genes expressed in the host cell. When possible, the polynucleotide encoding the heterologous polypeptide of interest may be modified to avoid predicted hairpin secondary mRNA structures.

There are known differences between the optimal translation initiation context nucleotide sequences for translation initiation codons in animals and plants and the composition of these translation initiation context nucleotide sequences can influence the efficiency of translation initiation. See, for example, Lukaszewicz et al. (2000) Plant Science 154:89-98; and Joshi et al. (1997); Plant Mol. Biol. 35:993-1001. As used herein, “translation initiation codon” means a codon that initiates translation of an mRNA transcribed from the nucleotide sequence of interest. As used herein, “translation initiation context nucleotide sequence” means an identity of three nucleotides directly 5′ of the translation initiation codon. In the present invention, the translation initiation context nucleotide sequence for the translation initiation codon of the polynucleotide nucleotide of interest, for example, the polynucleotide encoding a heterologous polypeptide of interest, may be modified to enhance expression in a plant, for example, duckweed. In one embodiment, the nucleotide sequence is modified such that the three nucleotides directly upstream of the translation initiation codon of the nucleotide sequence of interest are “ACC.” In a second embodiment, these nucleotides are “ACA.” Expression of a heterologous polynucleotide in a host plant, including duckweed, can also be enhanced by the use of 5′ leader sequences. Such leader sequences can act to enhance translation. Translation leaders are known in the art and include, but are not limited to, picornavirus leaders, e.g., EMCV leader (Encephalomyocarditis 5′ noncoding region; Elroy-Stein et al. (1989) Proc. Natl. Acad Sci USA 86:6126); potyvirus leaders, e.g., TEV leader (Tobacco Etch Virus; Allison et al. (1986) Virology 154:9); human immunoglobulin heavy-chain binding protein (BiP; Macajak and Sarnow (1991) Nature 353:90); untranslated leader from the coat protein mRNA of alfalfa mosaic virus (AMV RNA 4; Jobling and Gehrke (1987) Nature 325:622); tobacco mosaic virus leader (TMV; Gallie (1989) Molecular Biology of RNA, 23:56); potato etch virus leader (Tomashevskaya et al. (1993) J. Gen. Virol. 74:2717-2724); Fed-1 5′ untranslated region (Dickey (1992) EMBO J. 11:2311-2317); RbcS 5′ untranslated region (Silverthorne et al. (1990) J. Plant. Mol. Biol. 15:49-58); and maize chlorotic mottle virus leader (MCMV; Lommel et al. (1991) Virology 81:382). See also, Della-Cioppa et al. (1987) Plant Physiology 84:965. Leader sequence comprising plant intron sequence, including intron sequence from the maize alcohol dehydrogenase 1 (ADH1) gene, the castor bean catalase gene, or the Arabidopsis tryptophan pathway gene PAT1 has also been shown to increase translational efficiency in plants (Callis et al. (1987) Genes Dev. 1:1183-1200; Mascarenhas et al. (1990) Plant Mol. Biol. 15:913-920). See also the 5′ leader sequences from Lemna gibba RbcS genes, set forth as SEQ ID NOs:10-12 in U.S. Pat. No. 7,622,573; see also, GenBank Accession Nos. S45165 (SSU13; nucleotides 694-757), S45166 (SSU5A; nucleotides 698-755), and S45167 (SSU5B; nucleotides 690-751)).

In some embodiments of the present invention, nucleotide sequence corresponding to nucleotides 1222-1775 of the maize alcohol dehydrogenase 1 gene (ADH1; GenBank Accession Number X04049), or nucleotide sequence corresponding to the intron set forth as SEQ ID NO:7, 8, or 9 in U.S. Pat. No. 7,622,573, is inserted upstream of the polynucleotide encoding the heterologous polypeptide of interest within the expression construct, or upstream of the inhibitory polynucleotide within the auxotroph construct, to enhance expression of these operably linked polynucleotides.

It is recognized that any of the expression-enhancing nucleotide sequence modifications described above can be used in the present invention, including any single modification or any possible combination of modifications. The phrase “modified for enhanced expression” in a plant, for example, a duckweed plant, as used herein refers to a polynucleotide sequence that contains any one or any combination of these modifications.

### Signal Peptides

As noted above, in some embodiments of the invention, the expression constructs are used to produce a heterologous polypeptide of interest, which can be a secreted protein. Secreted proteins are usually translated from precursor polypeptides that include a “signal peptide” that interacts with a receptor protein on the membrane of the endoplasmic reticulum (ER) to direct the translocation of the growing polypeptide chain across the membrane and into the endoplasmic reticulum for secretion from the cell. This signal peptide is often cleaved from the precursor polypeptide to produce a “mature” polypeptide lacking the signal peptide. As such, a biologically active polypeptide can be expressed in a plant host cell from a polynucleotide sequence that is operably linked with a nucleotide sequence encoding a signal peptide that directs secretion of the polypeptide into the culture medium.

Plant signal peptides that target protein translocation to the endoplasmic reticulum (for secretion outside of the cell) are known in the art. See, e.g., U.S. Pat. No. 6,020,169. Any plant signal peptide can be used herein to target polypeptide expression to the ER. For example, the signal peptide can be an the Arabidopsis thaliana basic endochitinase signal peptide (amino acids 14-34 of NCBI Protein Accession No. BAA82823), the extensin signal peptide (Stiefel et al. (1990) Plant Cell 2:785-793), the rice α-amylase signal peptide (amino acids 1-31 of NCBI Protein Accession No. AAA33885), or a modified rice α-amylase signal sequence (see SEQ ID NO:17 in U.S. Pat. No. 7,622,573). In another embodiment, the signal peptide corresponds to the signal peptide of a secreted plant protein, for example, a secreted duckweed protein. The signal peptide also can correspond to a signal peptide of the secreted heterologous polypeptide.

Alternatively, a mammalian signal peptide can be used to target recombinant polypeptides expressed in a genetically engineered plant of the invention, for example, duckweed or other higher plant of interest, for secretion. It has been demonstrated that plant cells recognize mammalian signal peptides that target the endoplasmic reticulum, and that these signal peptides can direct the secretion of polypeptides not only through the plasma membrane but also through the plant cell wall. See U.S. Pat. Nos. 5,202,422 and 5,639,947 to Hiatt et al. In one embodiment of the present invention, the mammalian signal peptide that targets polypeptide secretion is the human α-2b-interferon signal peptide (amino acids 1-23 of NCBI Protein Accession No. AAB59402).

In one embodiment, the nucleotide sequence encoding the signal peptide is modified for enhanced expression in the plant host of interest, for example, duckweed, utilizing any modification or combination of modifications disclosed above for the polynucleotide sequence of interest.

The secreted heterologous polypeptide can be harvested from the culture medium by any conventional means known in the art and purified by chromatography, electrophoresis, dialysis, solvent-solvent extraction, and the like. In this manner, purified polypeptides, as defined above, can be obtained from the culture medium.

### Transgenic and Auxotrophic Plants

The class of plants that can be used in the methods of the invention is generally as broad as the class of plants amenable to transformation techniques, including both monocotyledonous (monocot) and dicotyledonous (dicot) plants. Examples of dicots include, but are not limited to, legumes including soybeans and alfalfa, tobacco, potatoes, tomatoes, and the like. Examples of monocots include, but are not limited to, maize, rice, oats, barley, wheat, members of the duckweed family, grasses, and the like. In some embodiments, the plant of interest is a member of the duckweed family of plants.

The term “duckweed” refers to members of the family Lemnaceae. This family currently is divided into five genera and 38 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdivlana); genus Spirodela (S. intermedia, S. polyrrhiza, S. punctata); genus Wolfia (Wa. angusta, Wa. arrhiza, Wa. australina, Wa. borealis, Wa. brasilliensis, Wa. columbiana, Wa. elongata, Wa. globosa, Wa. microscopica, Wa. neglecta); genus Wolfiella (Wl. caudata, Wl. denticulata, Wl. gladiata, Wl. hyalina, Wl. lingulata, Wl. repunda, Wl. rotunda, and Wl. neotropica) and genus Landoltia (L. punctata). Any other genera or species of Lemnaceae, if they exist, are also aspects of the present invention. Lemna species can be classified using the taxonomic scheme described by Landolt (1986) Biosystematic Investigation on the Family of Duckweeds: The Family of Lemnaceae—A Monograph Study (Geobatanischen Institut ETH, Stiftung Rubel, Zurich).

The term “duckweed nodule” as used herein refers to duckweed tissue comprising duckweed cells where at least about 50%, 55%, 60%, 65%, 70%, 75%, 80%, 85%, 90%, 95%, or 100% of the cells are differentiated cells. A “differentiated cell,” as used herein, is a cell with at least one phenotypic characteristic (e.g., a distinctive cell morphology or the expression of a marker nucleic acid or protein) that distinguishes it from undifferentiated cells or from cells found in other tissue types. The differentiated cells of the duckweed nodule culture described herein form a tiled smooth surface of interconnected cells fused at their adjacent cell walls, with nodules that have begun to organize into frond primordium scattered throughout the tissue. The surface of the tissue of the nodule culture has epidermal cells connected to each other via plasmadesmata. Members of the duckweed family reproduce by clonal propagation, and thus are representative of plants that clonally propagate.

The expression constructs and auxotrophic constructs for use in the methods of the present invention can be introduced into a plant or plant part of interest by any suitable method known to those of skill in the art. Transformation protocols as well as protocols for introducing polynucleotide constructs into plants may vary depending on the type of plant or plant cell or nodule, that is, monocot or dicot, targeted for transformation. Suitable methods of introducing polynucleotide constructs into plants or plant cells or nodules include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840, both of which are herein incorporated by reference), direct gene transfer (Paszkowski et al. (1984) EMBO J. 3:2717-2722), ballistic particle acceleration (see, e.g., U.S. Pat. Nos. 4,945,050; 5,879,918; 5,886,244; and 5,932,782 (each of which is herein incorporated by reference); and Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926). Other transformation protocols comprise contacting the plant with a virus or viral nucleic acids. Generally, one can incorporate the constructs described herein within a viral DNA or RNA molecule. Methods for introducing polynucleotides into and expressing a protein encoded therein, involving viral DNA or RNA molecules, are known in the art. See, for example, U.S. Pat. Nos. 5,889,191, 5,889,190, 5,866,785, 5,589,367 and 5,316,931.

Any plant tissue that can be subsequently propagated using clonal methods whether by organogenesis or embryogenesis, may be transformed with an expression construct and/or auxotrophic construct described herein. As used herein, “organogenesis” means a process whereby shoots and roots are developed sequentially from meristematic centers. As used herein, “embryogenesis” means a process by which shoots and roots develop together in a concerted fashion (not sequentially), whether from somatic cells or gametes. Exemplary tissues that are suitable for various transformation protocols described herein include, but are not limited to, callus tissue, existing meristematic tissue (e.g., apical meristems, axillary buds and root ineristems) and induced meristem tissue (e.g., cotyledon meristem and hypocotyl meristem), hypocotyls, cotyledons, leaf disks, pollen, embryos and the like.

The cells that have been transformed may be grown into plants in accordance with conventional ways (see, for example, McCormick et al. (1986) Plant Cell Reports 5:81-84) and assayed for the desired phenotypic trait, for example, an auxotrophic requirement for a component of a biosynthetic pathway for an essential compound.

The stably transformed duckweed utilized in this invention can be obtained by any method known in the art. In one embodiment, the stably transformed duckweed is obtained by one of the gene transfer methods disclosed in U.S. Pat. No. 6,040,498 to Stomp et al., herein incorporated by reference. These methods include gene transfer by ballistic bombardment with microprojectiles coated with a nucleic acid comprising the nucleotide sequence of interest, gene transfer by electroporation, and gene transfer mediated by Agrobacterium comprising a vector comprising the nucleotide sequence of interest. In one embodiment, the stably transformed duckweed is obtained via any one of the Agrobacterium-mediated methods disclosed in U.S. Pat. No. 6,040,498 to Stomp et al. The Agrobacterium used is Agrobacterium tumefaciens or Agrobacterium rhizogenes.

It is preferred that the stably transformed duckweed plants utilized in these methods exhibit normal morphology and are fertile by sexual reproduction. Preferably, transformed plants of the present invention contain a single copy of the transferred nucleic acid, and the transferred nucleic acid has no notable rearrangements therein. Also preferred are duckweed plants in which the transferred nucleic acid is present in low copy numbers (i.e., no more than five copies, alternately, no more than three copies, as a further alternative, fewer than three copies of the nucleic acid per transformed cell).

The present invention thus provides transgenic plants or plant parts having at least one auxotrophic requirement for an essential compound, such as an amino acid, fatty acid, carbohydrate, nucleic acid, vitamin, plant hormone, or precursor thereof. In some embodiments, the present invention provides transgenic plants and plant parts having an auxotrophic requirement for an amino acid. In one such embodiment, the transgenic plants or plant parts have an auxotrophic requirement for isoleucine that is caused by a targeted deletion, knockdown or interference of a threonine deaminase (TD). In other embodiments, the transgenic plants or plants parts have an auxotrophic requirement for glutamine that is caused by a targeted deletion, knockdown or interference of glutamine synthetase, either GS1, GS2, or both GS1 and GS2.

In other embodiments, the present invention provides transgenic plants and plant parts having an auxotrophic requirement for a vitamin such as biotin. In some of these embodiments, the transgenic plants or plant parts have an auxotrophic requirement for biotin that is caused by a targeted deletion, knockdown or interference of biotine synthase (BS).

In yet other embodiments, the present invention provides transgenic plants and plant parts having an auxotrophic requirement for a carbohydrate, nucleic acid, fatty acid, or plant hormone that is caused by a targeted deletion, knockdown, or interference of a component within a biosynthetic pathway for these essential compounds.

The following examples are offered by way of illustration and not by way of limitation.

### EXPERIMENTAL

### Example 1

### Genetic Engineering of a Lemna Isoleucine Auxotroph

Amino acids have fundamental roles both as building blocks of proteins and as intermediates in cellular metabolism. The ability of plants to synthesize the entire group of 20 amino acids is critical to their survival and thus can serve as an avenue for auxotroph development. The biosynthesis of isoleucine in plants takes place as part of the aspartic acid metabolic pathway where isoleucine is generated through the catabolism of threonine. Threonine deaminase (TD) is responsible for the conversion of threonine to 2-ketobutyrate, a key precursor in the isoleucine biosynthesis pathway. There is also evidence of an alternative pathway in which 2-ketobutyric acid is derived from methionine in times of osmotic stress via Met γ-lyase, however threonine appears to be the predominant precursor for isoleucine biosynthesis in plants. This conclusion is based on recent data showing that a T-DNA knockout mutant of Met γ-lyase did not alter the isoleucine concentration in leaves, flowers and seeds of Arabidopsis (Joshi and Jander (2009) Plant Physiol. 151:367-378). Further, this enzyme is predicted to be localized in the cytosol instead of the plastid where all isoleucine biosynthetic enzymes, downstream of 2-ketobutyric acid, are localized (Joshi et al. (2010) Amino Acids).

The following study describes the development of an isoleucine auxotroph platform in Lemna. This was accomplished by utilizing an RNAi based approach to knock down expression of a key enzyme, threonine deaminase, in the isoleucine biosynthetic pathway. The growth of these Lemna auxotroph lines is severely inhibited in normal conditions but fully recovered when supplemented with isoleucine. Since Lemna are grown via vegetative propagation within controlled growth rooms, exhibit naturally high levels of protein expression and have been genetically engineered to generate mammalian-like N-glycans, the addition of an auxotroph platform further advances this plant expression platform for the production of biotherapeutics and vaccines.

### Materials and Methods

### Reagents and Materials.

Lemna minor 8627 (Yamamoto et al. (2001) In Vitro Cell Dev Biol-Plant 37:5) was used for wild-type control and plant transformation experiments. All chemicals were obtained from Sigma-Aldrich. Recombinant DNA modification enzymes were obtained from New England Biolab. Thermal DNA polymerase was from Clontech (Titanium DNA polymerases) and Stratagene (PFUTurbo DNA Polymerase). Both TOP10 (Invitrogen) and Novablue E. coli competent cells were used for all DNA cloning.

Cloning of Full Length cDNA of Threonine Deaminase (TD).

A fragment of the TD cDNA was isolated by RT-PCR and subsequent nested PCR using dark grown RNA (4 and 24 hours in the dark) of L. minor (8627). Conserved regions of threonine deaminase from Arabidopsis, chickpea, rice, tobacco and tomato (Genbank Accessions AAL57674, CAA55313, P25306, AAG59585, AAK108849, XP_{—}469530, AAL58211, ABF98530 and NP_{—}001051069) were used in CODEHOP program (Rose et al. (2003) Nucleic Acids Res. 31:3763-3766) to design degenerate primers. The forward and reverse primers in the initial RT-PCR were BLX994 (5′-GCAGCCCGTGTTCTCCTTYAARYTNMG-3) (SEQ ID NO:25) and BLX996 (5′-TGGAAGAGGGWGATGTTCCANYKNGG-3) (SEQ ID NO:26). The subsequent forward and reverse nested primers were BLX995 (5′-CCGCCGGCAACCAYGCNCARGG-3′) (SEQ ID NO:27) and BLX997 (5′-GTGCAGCTGTCGAAGTYCATRTTNGCNCC-3′) (SEQ ID NO:28). Additional full length cDNA sequences were obtained following both 5′ and 3′ RACE using the SMART RACE cDNA Amplification Kit (Clontech). The gene specific primers for the 5′ and 3′ RACE were BLX1011 (5′-CTCGGCATAGGCGATAAGT-3) (SEQ ID NO:29) and BLX1010 (5′-GAGGCCCGATTCATGCCAT-3′) (SEQ ID NO:30), respectively. The nested primers for the 5′ and 3′ RACE were BLX1013 (5′-GCGGAATGAAAGTTCGGC-3′) (SEQ ID NO:31) and BLX1012 (5′-AGTATCCTCGAGCCAGCC-3) (SEQ ID NO:32), respectively. The following forward and reverse primers were used to amplify the full length cDNA (using the same RNA source mentioned above), BLX1030 (5′-CTCTCGGATCCTGCATCGTCTT-3′) (SEQ ID NO:33) and BLX1031 (5′-CAGAAGCCATAACACCGCATACA-3) (SEQ ID NO:34), respectively. This full length cDNA was cloned into pCR-Blunt II-TOPO (Invitrogen) to generate vector LmTD and its sequence was determined (SEQ ID NO:1).

### Construction of Plant Expression Vectors.

The threonine deaminase hairpin was created by cloning the 1300 base pairs (bp) fragment (nucleotides (nt) 371-1670 of SEQ ID NO:1) next to the 750 bp reverse complement (antisense) fragment (nt 371-1120 of SEQ ID NO:1). This hairpin is comprised of 750 bp stem and 550 bp loop regions. The first 1300 bp fragment was amplified from plasmid LmTD using primers BLX1045 (5′-TATGTCGACATGAAGGTCACACCCGACTC-3′) (SEQ ID NO:35) and BLX1046 (5′-TTCTAGACAAAATTITCAAACCCCATG-3) (SEQ ID NO:36), and it was cloned into pT7Blue (EMD Biosciences) via SalI and XbaI sites (underlined), to produce vector AUXC-T7-F. The second 750 bp antisense fragment was amplified from plasmid LmTD using BLX1047 (5′-TTCTAGACGCCATGGCATITGCATCGT-3′) (SEQ ID NO:37) and BLX1048 (5′-TGAGCTCATGAAGGTCACCACCGACTC-3) (SEQ ID NO:38), and it was cloned into AUXC-T7-F via XbaI and Sad sites (underlined) to produce vector AUXC-T7-FR. The SalI/SacI fragment from AUXC-T7-FR, containing the threonine deaminase hairpin, was cloned into the same sites in binary vector pBx53 (Gasdaska et al. (2003) Bioprocessing Journal 3:7), replacing the interferon alpha-2b sequence, to produce vector AUXC01 (FIG. 7). In this vector, the constitutive Superpromoter (Lee et al. (2007) Plant Physiol 145:1294-1300) drives the expression of the hairpin RNA molecule. The same hairpin fragment was cloned into a modified binary vector, similar to AUXC01, to produce vector AUXC02 (FIG. 8) in which the expression of the hairpin is driven by a strong constitutive Spirodela polyrrhiza polyubiquitin promoter (SpUbq; see SEQ ID NO:40 of the present application) (see also Cox et al. (2006) Nat. Biotechnol. 24:1591-1597; herein incorporated by reference in its entirety). Both vectors, AUXC01 and AUXC02, carry the aacCI gene for antibiotic selection with geneticin.

The codon-optimized hemagglutinin HA gene, derived from an avian influenza virus isolate A/chicken/Indonesia/7/2003 H5N1 (GenBank Accession No. AB030346; lacking the N-terminal 16 amino acids and internal amino acid residues 341-344), was synthesized and cloned into a modified pMSP-3 (Lee et al. (2007) Plant Physiol. 145:1294-1300) to produce vector MERB05 for selection with kanamycin. The Superpromoter/HA expression cassette was cloned into a modified version of AUXC02 to produce MERB06. The full-length SpUbq promoter in the MERB06 was replaced by the truncated SpUbq, containing only the first 117 bp (designated “SpUbq117” herein; see SEQ ID NO:41), to produce MERB07. Both MERB06 and MERB07 carry the aacCI gene for selection with geneticin.

### Plant Transformation and Screening of Transgenic Lines.

Transgenic Lemna plants were generated and maintained as previously described with a few modifications described below (Yamamoto et al. (2001) In Vitro Cell Dev. Biol. Plant 37:5). During the regeneration of fronds from callus, the geneticin concentrations were at 7.0 mg/L for AUXC01, 5.5, and 6.5 mg/L for AUXC02, and 6.0, 8.0, and 10.0 mg/L for MERB06 and MERB07. Transgenic plants were regenerated with isoleucine concentrations of 0.3 mM and 1.0 mM for each geneticin concentration in the initial two transformations (AUXC01 and AUXC02) and 0.3 mM in the subsequent transformations with MERB06 and MERB07 vectors. For the transformation of vector MERB05, calli were induced and maintained from auxotroph line AUXC02-B1-58 as previously described (Yamamoto et al. (2001) In Vitro Cell Dev. Biol-Plant 37:5) except that all media were supplemented with 0.3 mM isoleucine. MERB05 was transformed into this callus bank, and transgenic lines were generated from media containing 150, 200, and 250 mg/L kanamycin supplemented with 0.3 mM isoleucine.

Fronds were harvested into plant tissue culture containers (Greiner Bio-One, Frickenhausen, Germany; cat.#967164) containing 50 mL of SH medium (Schenk (1972) Can. J. Bot. 50:199-204) supplemented with 1% sucrose and 0.25 mM isoleucine for plants carrying the AUXC01 and AUXC02 vectors. Primary screening was conducted in 12-well multiwell tissue culture plates (Becton Dickinson, New Jersey, USA; Falcon Cat. #353225). Each well contained 3.5 ml of SH media with and without 0.25 mM isoleucine supplement. Two 3-frond clusters from each transgenic line were used to inoculate a pair of wells, and plants were grown for up to one month under continuous lighting. The temperature was maintained at 24° C. and the light intensity was kept at 220 μmol s⁻¹m⁻². Potential auxotroph lines underwent a secondary screen in 125 mL PET square media bottle (Nalgene cat. #342040-0125). Each bottle contained 50 mL of SH medium supplemented with or without 0.25 mM isoleucine. Each bottle was inoculated with three 3-frond clusters and was cultured for 14 days under continuous lighting in Percival growth chamber (Model 136LLX, Percival Scientific, IA, USA). The temperature and light intensity were 26° C. and 620 μmol s⁻¹m⁻², respectively. Plant lines regenerated from MERB05, MERB06 and MERB07 transformations were evaluated directly in the secondary screening format with SH medium supplemented with 0.375 mM isoleucine. All subsequent experiments were performed in the presence of 0.375 mM isoleucine in square bottles and with the same conditions as in the secondary screen.

### Quantitative Real-Time PCR.

After 14 days of growth in the presence of 0.25 mM isoleucine, 100 mg of tissues were harvested, flash frozen in liquid nitrogen, and homogenized using a FastPrep FP120 (Bio101). Total RNA was extracted from the supernatant using the RNeasy Plus Mini Kit (Qiagen) according to manufacturer's protocol. First strand cDNA was synthesized from 1 μg of total RNA using the iScript cDNA Synthesis Kit (Bio-Rad) according to manufacturer's protocol. Following the first strand cDNA synthesis, the reaction volume was adjusted to 100 μL, and one μL was used as a template in the real-time PCR using iQ SYBR Green Supermix (Bio-Rad). The real-time PCR was performed using the Bio-Rad iCycleriQ Multicolor Real-time PCR Detection System. The 3′ terminal 135 bp region of the threonine deaminase full length cDNA was selected as a target for Real-time PCR in order to avoid amplification of the threonine deaminase sequence present in the hairpin RNA molecule. The forward and reverse primers used in the real-time PCR are BLX1161 (5′-TGCCCTAGAGATGTCCAACAAGG-3′) (SEQ ID NO:39) and BLX1031 (described above), respectively. The endogenous histone gene was also amplified in parallel, and it was used as a reference to normalize loading. Each sample, including the histone reference control, was run in duplicate on the PCR plate, and reported data are the average of two separate real-time PCR runs.

### Hemagglutinin (HA) Activity Assay.

The expression level of HA in isoleucine auxotroph lines transformed with MERB05, MERB06, and MERB07 was determined according to standard hemagglutination assay. Tissues (100 mg) were homogenized in 1 mL of extraction buffer in FastPrep FP120 (Bio101), and 50 μL of the supernatant was serial diluted 2-fold into Nunc U-bottom 96-well plates. Then, 50 μl of 10% turkey red blood cell (Fitzgerald Industries International Inc., Concord, Mass.) or chicken red blood cells was added and incubated for 1 hr at room temperature. Negative controls included Lemna wild type and PBS, and positive control included recombinant Avian Influenza H5 hemagglutinin of A/Vietnam/1203/2004 (Protein Sciences Corporation, Meriden, Conn.). The plate was scored visually for a partial (partial button formation) or complete (cloudy solution with no button formation) hemagglutinin activity. If there is no hemagglutinin activity in the sample, then a well defined button would be formed with clear solution.

### Results

Isolation of Threonine Deaminase cDNA from Lemna minor

Amino acid sequence alignments were performed with publically available threonine deaminase protein sequences from several plant species including Arabidopsis thaliana, Cicer arietinum, Nicotiana attenuata, Oryza sativa and Solanum lycopersicum. Highly conserved regions were identified and used to facilitate isolation of Lemna threonine deaminase cDNA using RT-PCR and RACE PCR methods. A full-length cDNA of the threonine deaminase gene (Lemna minor TD isoform #1 (designated LmTD); see SEQ ID NO:1) was isolated which consists of 2088 bp, contains an open reading frame of 1959 bp and encodes for a protein of 653 amino acids. The 5′ and 3′ UTRs of this clone are 40 bp and 89 bp, respectively. Additionally, a second cDNA isoform, L. minor TD isoform #2 (see SEQ ID NO:4 for full-length cDNA, SEQ ID NO:6 for predicted amino acid sequence) was isolated, which showed a 99.7% nucleotide sequence identity and 99.6% nucleotide identity to LmTD in the region of overlap. BLASTP analysis performed with the predicted amino acid sequence of LmTD (see SEQ ID NO:3) against the GenBank Non-redundant protein database showed that these sequences are most homologous to the plant threonine deaminases. LmTD showed the highest percent amino acid identity with plant threonine deaminases from Arabidopsis thaliana (GenBank Accession No. AAL57674), Oryza sativa (GenBank Accession No. NP_{—}001051069) and Nicotiana attenuate (GenBank Accession No. AAG59585) with 67%, 71%, and 56% amino acid identity, respectively. Lemna threonine deaminase protein sequence was analyzed by TargetP (Emanuelsson et al. (2000) J. Mol. Biol. 300:1005-1016; Nielsen et al. (1997) Protein Eng. 10:1-6) and was predicted to contain a chloroplast transit peptide of 30 amino acids in length. This result is consistent with TD from other plants in which they are known to be localized to the chloroplast (Singh et al. (1995) Plant Cell 7:935-944; Samach et al. (1991) Proc. Natl. Acad. Sci. U.S.A. 88:2678-2682.).

### Construction of RNAi Vectors and Development of the Transformation Methods

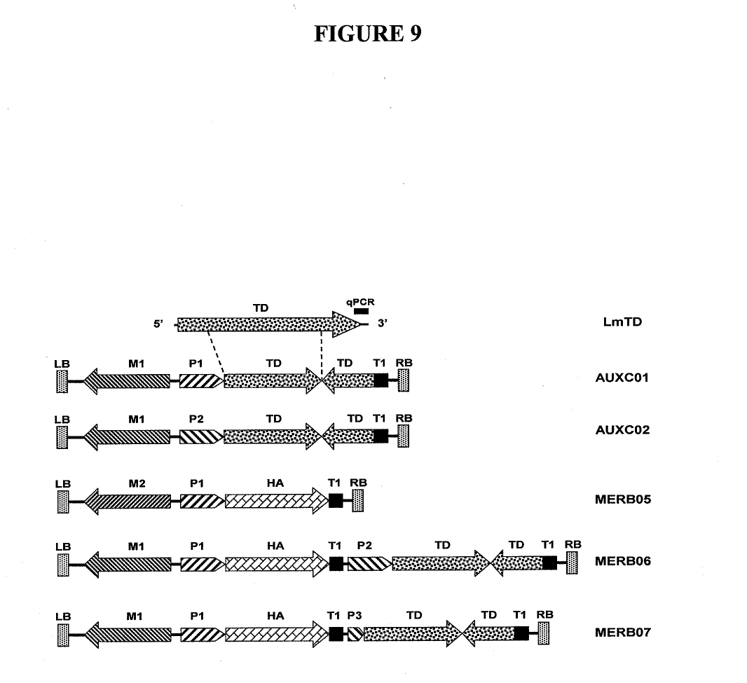

A strategy similar to the RNAi-based silencing of Lemna xylosyltransferase and fucosyltransferase genes (Cox et al. (2006) Nat. Biotechnol. 24:1591-1597), was employed to knockdown expression of threonine deaminase (TD). The hairpin RNA molecule for TD was designed with a stem of 750 bp and a loop of 550 bp. The first portion (stem and loop; sense orientation) contains 1300 bp (nt 371-670 of SEQ ID NO:1) all of which resides within the coding region of the gene. This gene fragment is fused to the second portion (stem only, antisense orientation), which contains 750 bp (nt 371-1120 of SEQ ID NO:1). A schematic of this hairpin construct is shown in FIG. 5. Expression of the TD hairpin RNA molecule was evaluated in Lemna with two independent expression vectors, AuxC01 and AuxC02, which facilitate constitutive, high expression via the chimeric Superpromoter and Spirodela polyrrhiza ubiquitin (SpUbq) promoter, respectively (see also FIG. 9).

In order to determine the appropriate concentration of isoleucine required to rescue auxotroph tissue during transformation, the isoleucine tolerance of wild-type Lemna minor was evaluated with fronds grown for 8 days in the absence (0 mM isoleucine) or in the presence of a range of isoleucine concentrations (0.05, 0.1, 0.3, 0.6, or 1.0 mM isoleucine). Lemna minor fronds can tolerate up to 1.0 mM isoleucine while the ideal concentration is 0.3 mM. Lemna callus tissue was also evaluated in the absence (0 mM isoleucine) or the presence of isoleucine (0.3 or 1.0 mM) for 6 weeks on Frond Regeneration Medium, without antibiotic selection, to mimic the plant transformation conditions that would be used to generate transgenic lines with AuxC01 and AuxC02. Callus tissue was able to multiply and differentiate normally in media containing either 0.3 mM or 1.0 mM isoleucine.

Transgenic lines were generated with AuxC01 and AuxC02 using standard Agrobacterium transformation methods as previously reported (Cox et al. (2006) Nat. Biotechnol 24:1591-1597) and detailed in Table 3 below. All regenerated fronds were harvested into SH medium containing 0.25 mM isoleucine to rescue plants with reduced levels of threonine deaminase. A total of 126 transgenic lines were regenerated from AUXC01 and AuxC02 transformations (Table 3). Transgenic plants were regenerated at similar times from plates containing 0.3 mM or 1.0 mM isoleucine (in both AUXC01 and AUXC02 vectors) however, more plants were regenerated from 0.3 mM than from 1.0 mM isoleucine, which is consistent with results observed from the isoleucine tolerance experiments described above. The total number of transgenic lines generated from 0.3 mM and 1.0 mM isoleucine in AUXC01 were 80 and 46, respectively, and in AUXC02 were 37 and 21, respectively.

TABLE 3
Transformation conditions and screening for auxotroph lines

| | | | | Auxotroph | |
|---|---|---|---|---|---|
| | Isoleucine | Geneticin | Lines | after 2^{nd} | % |
| Vector | (mM) | (mg/L) | generated | screen | Auxotroph^{a} |
| AUXC01 | 0.3 | 7 | 80 | 2 | 1.6% |
| AUXC01 | 1 | 7 | 46 | 0 | 0% |
| AUXC02 | 0.3 | 5.5 | 26 | 2 | 3.4% |
| AUXC02 | 0.3 | 6.5 | 11 | 6 | 10.3% |
| AUXC02 | 1 | 5.5 | 10 | 4 | 6.9% |
| AUXC02 | 1 | 6.5 | 11 | 5 | 8.6% |

^{a}The percentage is calculated relative to the total number of each vector.

### Screening and Identification of Isoleucine Auxotrophs

Transgenic Lemna minor plant lines were initially screened in 12-well plates in the presence and absence of isoleucine to identify auxotroph candidates. Plant lines that demonstrated reduced growth, inability to propagate and/or poor plant health were scored as potential auxotrophs. Three transgenic lines (AUXC02-B1-7, 8, and 9) and wild-type Lemna minor were grown for 15 days in 12-well plate containing SH media supplemented with 0 (−Ile) and 0.25 mM (+Ile) isoleucine. In this primary screen, lines AUXC02-B1-7 and AUXC02-B1-8 were scored as auxotrophs while line AUXC02-B1-9 exhibited a phenotype similar to the Lemna minor wild-type control. Following the initial 12-well screen, potential auxotroph lines were put through secondary screening using 0.1 mM and 0.25 mM isoleucine in larger growth vessels (Table 3 above). Two transgenic lines (AUXC02-B1-19 and 58) and wild-type Lemna minor were grown for 13 days in PET square media bottle containing SH medium supplemented with 0, 0.1 mM, or 0.25 mM isoleucine. From the secondary screen, the AuxC01 plant lines yielded two auxotrophs (1.6%) while AUXC02 produced a total of 17 auxotrophs (29.2%). Transgenic lines AUXC02-B1-19 and AUXC02-B1-58 both exhibited strong auxotroph phenotypes and proportional biomass increase with elevated levels of isoleucine supplement indicating that the phenotype is specific to the isoleucine biosynthesis. Isoleucine auxotrophs were generated equally from either 0.3 mM (10 lines) or 1.0 mM (9 lines) isoleucine indicating that 0.3 mM is sufficient in rescuing plants carrying the threonine deaminase (TD) RNAi construct. Following secondary screening, five of the top isoleucine auxotroph lines, AUXC02-B1-7,8,19, 33, and 58, were selected for further analysis.

### Growth Optimization of Isoleucine Auxotrophs

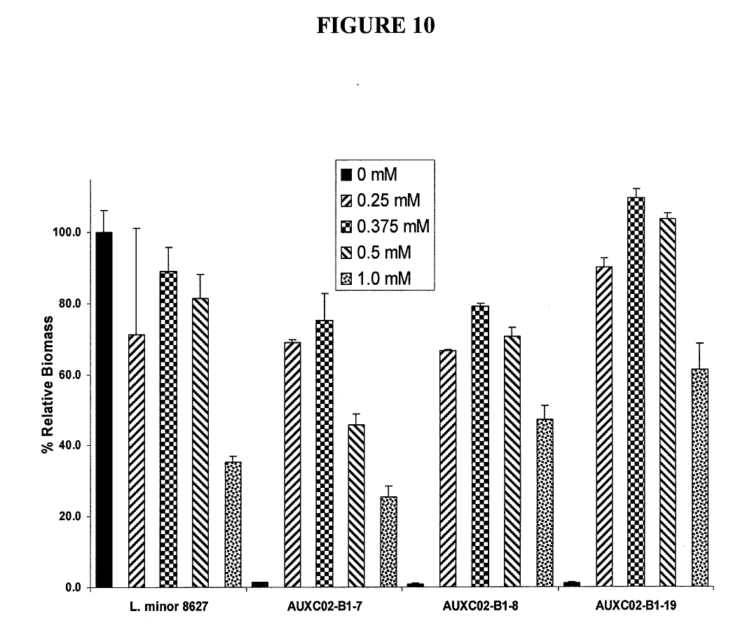

Further experiments were conducted on the selected AUXC plant lines to determine their tolerance level to isoleucine supplementation and determine the optimal conditions needed to restore biomass accumulation to wild-type levels. Selected auxotroph lines were grown in media supplemented with 0, 0.25, 0.375, 0.5, and 1.0 mM isoleucine (FIG. 10) where all of the auxotroph lines exhibited the highest biomass accumulation in media supplemented with 0.375 mM. In the absence of isoleucine, the lines were completely unable to propagate and with isoleucine concentrations of 1.0 mM experienced a dramatic reduction in biomass yield. Isoleucine concentrations of 0.25 mM and 0.50 mM resulted in a slight drop in biomass yield. These results are consistent with initial experiments where the growth of wild type plants was inhibited by 0.6 mM and 1 mM isoleucine. Most importantly, auxotroph line AuxC02-B1-19 showed complete restoration of biomass accumulation, exhibiting higher yields than wild-type Lemna, at concentrations of 0.375 mM and 0.50 mM isoleucine.

These selected auxotrophic lines were further evaluated to determine optimal light intensity. The plant lines were grown under three levels of light (340, 480, and 630 mol*m⁻²*s⁻¹) in media containing 0.375 mM isoleucine, where the optimal light intensity was determined to be 630 μmol*m⁻²*s⁻¹. The improvement in biomass accumulation for the plants grown under 630 μmol*m⁻²*s⁻¹ light was not dramatic, with only 6% and 7% increase compared to that at 480 μmol*m⁻²*s⁻¹ and 340 μmol*m⁻²*s⁻¹, respectively. Based on the data from these experiments the optimal growth conditions for AUXC transformants were determined to be 0.375 mM isoleucine and 630 μmol*m⁻²*s⁻¹.

### Reduced Threonine Deaminase RNA Level in Auxotrophic Lines

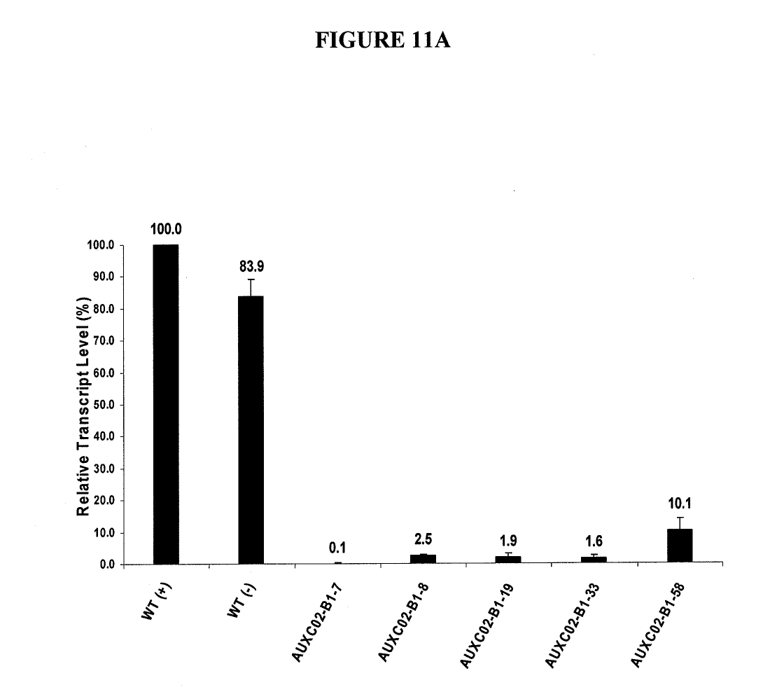

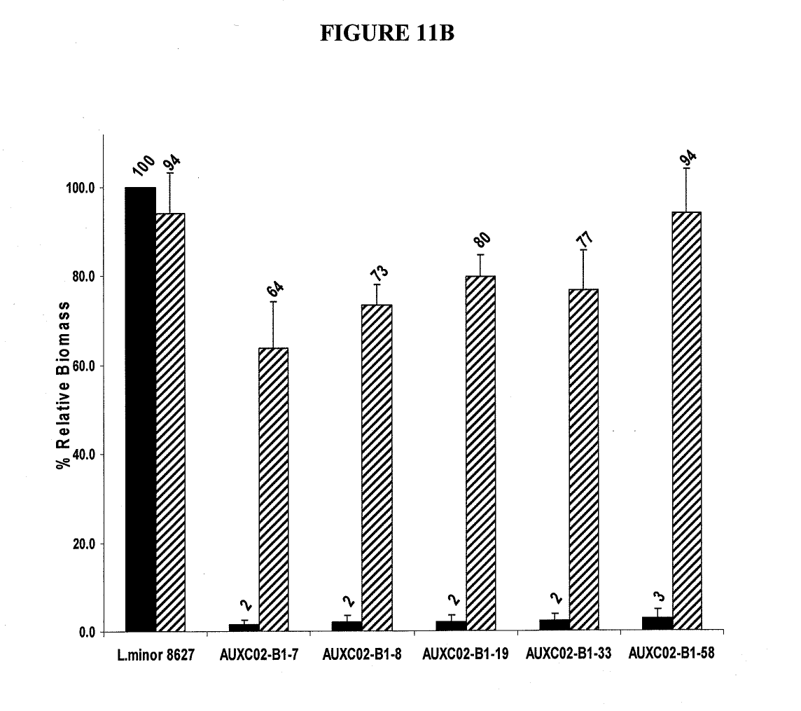

Quantitative real-time RT-PCR was employed to confirm that the phenotype observed in the top isoleucine auxotroph lines correlated with reduced mRNA levels of threonine deaminase (TD). In order to avoid amplifying the TD sequences present in the AuxC RNAi constructs, a region in the 3′ end of the TD gene, not present in the hairpin RNA molecule, was selected (see general diagram in FIG. 9). Two real-time PCR experiments were performed, using cDNA derived from five auxotroph lines, and the averaged results are shown in FIG. 11. For these experiments all auxotroph lines were grown in the presence of 0.25 mM isoleucine and relative mRNA transcript levels were calculated relative to the wild type Lemna grown in the presence of isoleucine which was set to 100%. All five of the isoleucine auxotroph lines showed a significant reduction in the threonine deaminase mRNA level (FIG. 11A) and corresponding inability to propagate in the absence of isoleucine (FIG. 11B). Line AUXC02-B1-7 had the most knockdown of its TD mRNA, with only 0.1% of the transcripts of the wild-type control. Interestingly, a 90% reduction in threonine deaminase mRNA level was sufficient to generate the isoleucine auxotroph phenotype as shown in line AUXC02-B1-58 and also allowed for full recovery of biomass accumulation. Real-time PCR results obtained from eight additional auxotroph lines showed that the RNA level in all of the auxotroph lines ranged from 0.1% to 10.1% of the wild-type control. However, the RNA level in most of these lines clustered around 2% of the wild-type level and in all cases demonstrated the association of the auxotroph phenotype and reduced TD mRNA level.

Rescue of Isoleucine Auxotrophs with 2-Ketobutyrate and Long Term Stability

The specificity of the auxotroph phenotype was further evaluated by growing Lemna minor auxotroph lines AUXC02-B1-19 and AUXC02-B1-58 in the presence of 2-ketobutyrate (2-KB), leucine (Leu), and glutamine (Gln). Given that 2-KB is the key intermediate product formed by TD in the conversion of threonine to isoleucine, it was expected to facilitate recovery of the auxotroph lines. Leu and Gln were utilized as auxiliary controls to demonstrate the effect of non-related amino acids on the recovery of the auxotroph lines. Similar results were obtained for both of the selected auxotroph lines; therefore, results only from line AUXC02-B1-19 are discussed here. Auxotroph line AUXC02-B1-19 was grown in SH media supplemented with various amino acids for 14 days, as follows: no supplement; 0.375 mM isoleucine (Ile); 1 mM 2-ketobutyric acid; 0.375 mM glutamine (Gln); or 0.375 mM leucine (Leu). Wild-type Lemna minor with no supplement served as a control. As with previous experiments, the auxotroph lines were not able to grow in the absence of Ile supplement, and could be rescued in the presence of 0.375 mM Ile. The addition of 1.0 mM 2-KB to the growth media resulted in full recovery of the auxotroph lines while the concentration of 0.375 mM 2-KB allowed for only partial rescue. Concentrations of 3.0 mM and 12.0 mM 2-KB were also evaluated and determined to severely inhibit the growth of wild-type Lemna plants. As predicted, the presence of either Leu or Gln supplements was not sufficient to rescue the auxotroph lines.

This experiment also demonstrated the genetic stability of the RNAi derived auxotroph phenotype for the two top auxotroph lines over a prolonged period of time. These plant lines continued to exhibit the auxotroph phenotype and ability for full recovery 2.5 years after the initial line harvest.

### Expression of Recombinant AIV HA in Isoleucine Auxotroph Platform

In order to validate the isoleucine auxotroph platform for expression of recombinant proteins, the avian influenza hemagglutinin (AIV HA) gene (isolate A/chicken/Indonesia/7/2003 (H5N1)) was selected for expression. Two methods were employed for expression of AIV HA in the isoleucine auxotroph platform. The first method was to re-transform one of the top isoleucine auxotroph lines (AuxC02-B1-58) with a transformation vector containing an AIV HA expression cassette (MERB05, see FIG. 9). This required the creation of a callus bank from frond tissue of plant line AUXC02-B1-58, subsequent transformation with the MERB05 vector and selection for kanamycin resistant plants. The second method involved the co-expression of both the TD hairpin RNA molecule and AIV HA within the same vector (MERB06 and MERB07, see FIG. 9). Given the success of the AuxC02 transformations, the SpUbq promoter was selected to drive expression of the TD hairpin RNA molecule with a full-length promoter version (SpUbq; see SEQ ID NO:40) and truncated promoter version (SpUbq117; see SEQ ID NO:41), within transformation vectors MERB06 and MerB07, respectively.

Transgenic lines were generated with MERB05, MERB06, and MERB07 and screened for the auxotroph phenotype as described above. The results of these transformations are detailed in Table 4 below. Not surprisingly, MERB05 re-transformed into the AUXC02-B1-58 isoleucine auxotroph background, generated the most isoleucine auxotrophs at 83% (25/30). Of the remaining MERB05 transformants that did not exhibit the auxotroph phenotype, four of these five lines showed growth inhibition in both the presence and absence of isoleucine supplement, suggesting a negative effect from transgene integration. Overall the results from the MERB05 transformation are very promising in that the RNAi silencing of TD is very stable in transgenic Lemna fronds and remains stable throughout the different phases of the tissue culture process. In similar fashion to their predecessor AuxC02, the MERB06 and MERB07 transformations successfully generated auxotroph lines with MERB06 and MERB07 producing 23% and 56% auxotrophs, respectively (Table 4).

TABLE 4
Expression of HA and threonine deaminase RNAi

| | | | | | | Auxotroph |
|---|---|---|---|---|---|---|
| | | | | | | with |
| | Lines | Auxotroph | Undetectable | Detectable | High | HA activity |
| Vector | generated | (%) | activity (%) | activity (%) | activity (%) | (%) |
| MERB05 | 30 | 25 (83) | 26 (87) | 4 (13) | 0 (0) | 3 (10) |
| MERB06 | 39 | 9 (23) | 39 (100) | 0 (0) | 0 (0) | 0 (0) |
| MERB07 | 18 | 10 (56) | 9 (50) | 8 (44) | 1 (6) | 6 (33) |

Following the primary screen and identification of potential auxotroph lines, transgenic lines were subsequently screened for expression of AIV HA via the hemagglutination activity assay (Table 4). Four MERB05 lines and nine MERB07 lines showed measurable expression of AIV HA while all of the MERB06 plant lines showed no measurable HA activity. The lack of HA activity demonstrated in several of these transgenic lines is likely due to the limit of detection of the HA assay. Three out of the four MERB05 lines expressing AIV HA were also isoleucine auxotrophs compared to six out of nine for MERB07. The best results were obtained from transgenic line MERB07-B1-4 which demonstrated high HA activity, a strong auxotroph phenotype in the absence of isoleucine supplement and the ability for full biomass recovery with isoleucine supplementation (0.375 mM Ile). Overall, these results provide proof of concept for expression of recombinant proteins in the Lemna isoleucine auxotroph platform.

### Discussion

The interaction and regulation of the aspartate metabolic pathway is quite complex with many end products (isoleucine, threonine, methionine, and lysine) and feedback mechanisms. For example, aspartate kinase is the first enzyme in this metabolic pathway and is directly inhibited by three of its four end products, threonine, lysine, and S-adenosylmethionine. In this study, a negative effect on growth of Lemna was observed with an isoleucine concentration of 1.0 mM. This may be attributed to indirect feedback inhibition of aspartate kinase via the threonine deaminase route, since an elevated threonine level would inhibit aspartate kinase and eventually limit the synthesis of methionine and lysine. In addition to the well-known feedback regulation of isoleucine at the enzyme level, the reduction of TD mRNA levels in wild-type Lemna grown in 0.25 mM isoleucine (as determined by quantitative RT-PCR analysis) suggests that some feedback regulation may also exist at the transcriptional level. There is evidence of an alternative pathway of isoleucine biosynthesis in which 2-ketobutyric acid is derived from methionine in times of osmotic stress via Met γ-lyase; however, threonine appears to be the predominant precursor for isoleucine biosynthesis in Lemna.

This study demonstrates the development of an isoleucine auxotroph platform in Lemna via RNAi-mediated targeting of TD within in the isoleucine biosynthetic pathway. Several lines of evidence support the assertion that the isoleucine auxotroph plants are the result of the specific knock down of this target enzyme. First the isolated Lemna TD cDNA has the highest sequence homology to known TD genes (Arabidopsis, N. attenuate, and rice) in the GenBank database. Additionally, supplementation of either 2-KB or isoleucine is required for survival of auxotroph plant lines, and quantitative RT-PCR analysis reveals ≧90% reduction in the endogenous TD mRNA in the auxotroph lines. Furthermore, isoleucine supplementation is dosage dependent, where higher isoleucine levels result in increased growth up to the level of wild-type tolerability while other amino acids were not adequate for rescue.

The effectiveness of the RNAi strategy in previous unpublished experiments and in this study suggests that the expression level of the hairpin RNA molecule is a factor for consideration. Transient expression studies with the J-glucuronidase gene (GUS) reveal that the relative strength of the promoters used in this study in decreasing order are: SpUbq (full length; SEQ ID NO:40), SpUbq117 (truncated version; SEQ ID NO:41), and Superpromoter. The high percentage of auxotrophs generated from the AUXC02 vector (SpUbq promoter) as compared to the AUXC01 vector (Superpromoter) suggests that a higher expression level of the hairpin RNA molecule was needed for sufficient TD knock down and generation of the desired auxotroph phenotype.

Quantitative real-time RT-PCR data obtained from the top five isoleucine auxotrophs showed that >90% of the target mRNA was eliminated. Transgenic line AUXC02-B1-58, which demonstrated the least suppression of the top auxotroph lines (10.1% of wild-type TD mRNA level), was capable of full recovery under optimal growth and isoleucine supplementation conditions. Similar results were shown with transgenic line AUXC02-B1-19, which demonstrated 1.9% of wild-type TD mRNA level and full biomass recovery. The auxotroph line with the most potent mRNA knock down, AUXC02-B1-7 with 0.1% of wild-type TD mRNA level, was only capable of ˜80% biomass recovery with isoleucine supplementation, suggesting that there is an ideal range of RNAi suppression needed to allow for full biomass recovery. Similar results were obtained with several other auxotroph lines in this study, where dramatic suppression of TD mRNA levels resulted in only partial recovery of plant biomass yield. An increase in the isoleucine concentration of the growth media was not sufficient for these plant lines to fully overcome the most potent RNAi suppression, likely due to the complex feedback regulation (with other amino acids) within the aspartate pathway.

The co-expression of H5N1 HA with the TD hairpin RNA molecule did not appear to alter the effect of the RNAi knock down since a similar frequency of auxotroph lines were obtained from AUXC02 (29%) and MERB06 (23%) vectors. The slight reduction in RNAi expression produced by the truncated SpUbq117 promoter (MERB07) proved to be more effective than the full-length SpUbq (MERB06) promoter in generating isoleucine auxotroph lines. This is further evidence that an optimal range of RNAi expression allows for sufficient knock down of endogenous TD expression and subsequent generation of the desired auxotroph phenotype. In addition, the high frequency of auxotrophs generated from MERB07 was accompanied by a higher frequency and expression level of HA protein.

The successful regeneration of many isoleucine auxotrophs following MERB05 transformation demonstrated that the stability of this auxotroph phenotype is not limited to differentiated plants, but it was also extended into the tissue culture phase with dedifferentiated callus tissue. The RNAi-mediated silencing of the endogenous TD gene was shown to remain genetically stable over time (2.5 years or more) in transgenic auxotroph plant lines and throughout the different phases of the tissue culture process. This demonstrated genetic stability is an important component of the auxotroph platform and illustrates the utility as a reliable biocontainment system.

An avian influenza HA protein was successfully produced in the Lemna auxotroph platform. There is some flexibility in this system since one can choose to use either the sequential transformation or the co-transformation of both genes (HA and the TD RNAi) within the same vector. To further expand the auxotroph repertoire, this RNAi strategy may be used to target enzymes involved in the biosynthesis of other amino acids, vitamins, cofactors, and other essential compounds in plants, as exemplified herein below for the amino acid glutamine and the vitamin biotin.

### Example 2

### Genetic Engineering of Lemna Glutamine Auxotroph

An RNAi approach similar to that described in Example 1 was utilized to engineer a Lemna glutamine auxotroph. cDNAs encoding two isoforms of a cytosolic glutamine synthetase (GS1) and two isoforms of a plastid-localized glutamine synthetase (S2) were cloned from Lemna minor using degenerate primer PCR with primers designed from amino acid sequence alignments of published GS sequences from other plant species. The full-length cDNAs for L. minor GS1 isoform #1 and L. minor GS1 isoform #2 are set forth in SEQ ID NOs:7 and 10, respectively. The full-length cDNAs for L. minor GS2 isoform #1 and L. minor GS2 isoform #2 are set forth in SEQ ID NOs:13 and 16, respectively.

A chimeric RNAi construct was designed based on the cDNAs for GS1 isoform #1 (SEQ ID NO:7) and GS2 isoform #1 (SEQ ID NO:13). A schematic showing this RNAi construct is shown in FIG. 6. This chimeric RNAi construct was cloned into two different vectors to generate the AUXD01 (FIG. 12) and AUXD02 (FIG. 13) RNAi vectors. The AUXD01 RNAi vector uses the Superpromoter to drive expression of the chimeric RNAi construct. The AUXD02 RNAi vector uses the Spirodela polyrrhiza (SpUbq; SEQ ID NO:40) promoter to drive expression of this chimeric RNAi construct.

Transgenic Lemna plants were generated and maintained in a manner similar to that described above. Following Agrobacterium-mediated transformation, Agrobacterium co-cultivation was performed. Lemna nodules were then maintained on selection medium with varying levels of geneticin and auxotrophic supplement as shown in Table 5 below.

TABLE 5
GS RNAi Transformation and Selection Conditions.

| | | Selection (mg/L | |
|---|---|---|---|
| Genes Targeted | Construct | Geneticin) | Glutamine (mM) |
| GS1 and GS2 | AUXD01 | 5.5, 6.5, 7.0 | 10.0, 25.0 |
| GS1 and GS2 | AUXD02 | 5.5, 6.5 | 10.0, 25.0 |

Transgenic plant lines were successfully generated from AUXD01 and AUXD02 transformations. After harvest, plant lines were maintained in standard Lemna growth medium with appropriate supplementation with 10.0 mM glutamine. The number of lines generated per transformation are shown in Table 6 below.

TABLE 6
Glutamine Synthetase Auxotrophic Lines Generated Per Transformation

| Auxotrophic | | |
|---|---|---|
| Requirement | Construct | Total Lines Generated |
| Glutamine | AUXD01 | 29 |
| Glutamine | AUXD02 | 33 |

A primary screening process similar to that described in Example 1 was carried out to evaluate the phenotype of these auxotroph plant lines. Plant lines were initially screened in 12-well plates where auxotroph transformants were grown with and without glutamine supplementation. Lines that exhibited poor growth in standard media and subsequent recovery in the presence of supplement were selected for secondary screening. For secondary screening the standard format was used with plant lines being grown in IV's for 14 days.

### Results.

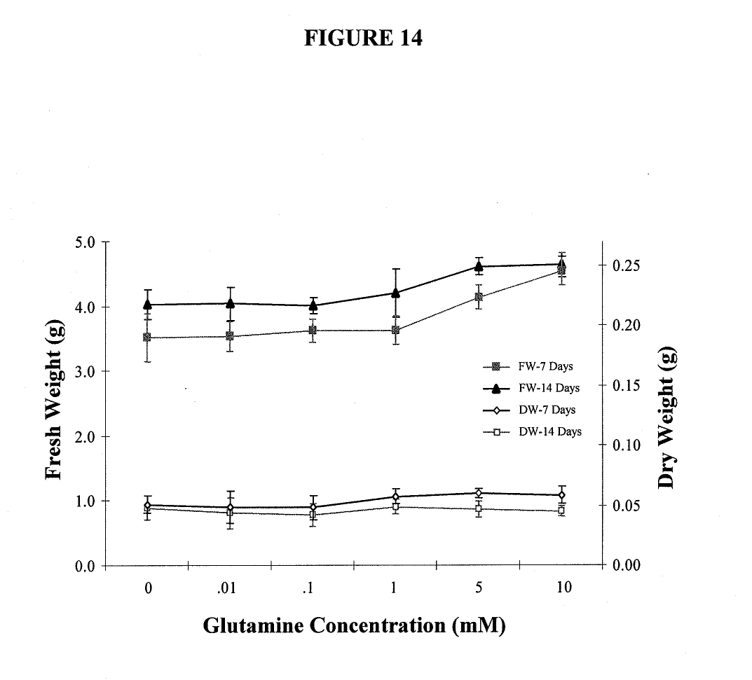

Initial experiments were conducted to determine the tolerance range of wild-type Lemna minor plants to glutamine. As shown in FIG. 14, glutamine alone had no effect on accumulation of plant biomass in wild-type plants after seven days.

Primary screening of Lemna minor auxotrophic AuxD01 and AuxD02 transformants grown with (+) and without (−) 0.25 mM glutamine, when compared to similarly treated wild-type Lemna minor plants, showed several lines with the desired auxotrophic phenotype. The auxotrophic plants grown in the absence of glutamine were unable to grow and had very poor plant health—these lines were essentially not able to survive in the absence of glutamine.

These results were confirmed with secondary screening in IV's for Lemna minor AUXD01 transformants (including screening of lines 2, 17, 9, and 4) and Lemna minor AUXD02 transformants (including screening of lines 29, 31, and 32). Auxotrophic Lemna minor plant lines transformed with the AUXD01 vector grown with 0.0 mM, 0.1 mM, or 0.25 mM isoleucine in the growth medium compared to wild-type plants. Auxotrophic Lemna minor plant lines transformed with the AUXD02 vector grown with 0 mM, 10 mM, or 30 mM glutamine in the growth medium compared to wild-type plants. Secondary screening of AUXD01 transformants in the presence of 0.25 mM glutamine in the growth medium showed that the plants exhibited almost a full recovery to a wild-type phenotype. Similar results were observed in the secondary screening of AUXD02 transformants. In the absence of isoleucine, these plants exhibited poor growth and plant health.

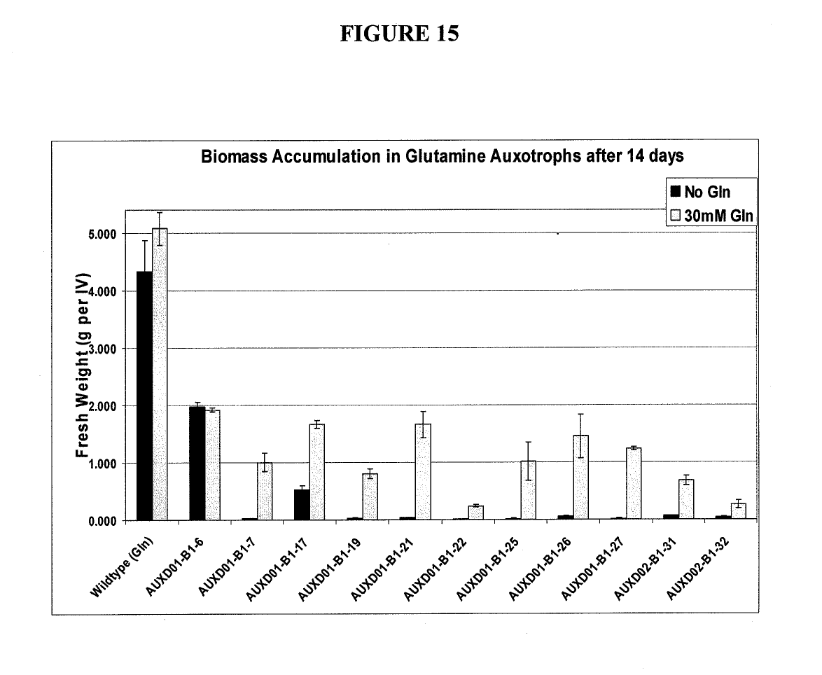

As with the isoleucine auxotroph lines, several AUXD01 and AUXD02 lines were further characterized to measure changes in fresh weight in the presence and absence of 30 mM glutamine (FIG. 15). Auxotroph lines showed a significant increase in fresh weight in the presence of glutamine supplementation.

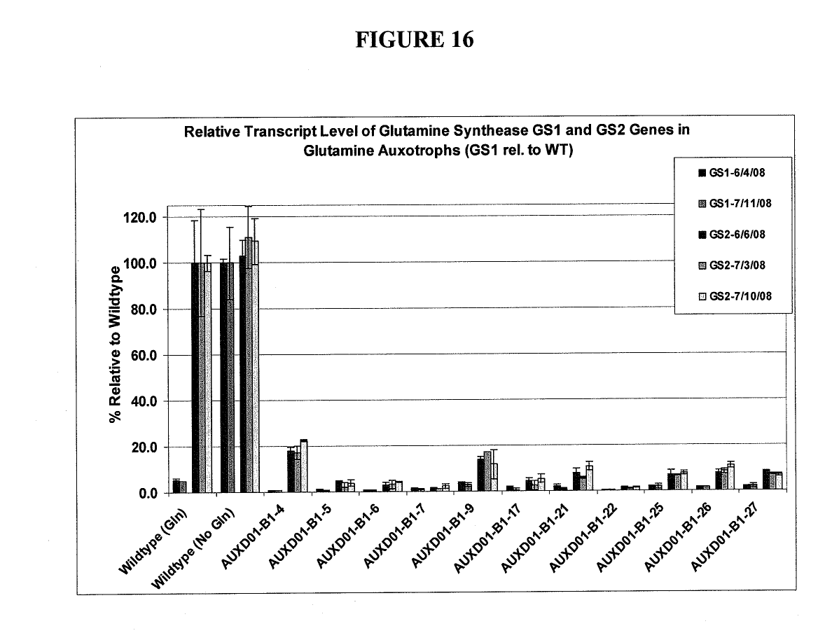

To confirm that the RNAi construct targeted endogenous GS, GS1 and G2 mRNA transcript levels were analyzed by qPCR in several of the auxotrophic lines. GS mRNA levels were significantly attenuated in the AUXD01 and AUXD02 lines (FIG. 16). Interestingly, in wild-type plants, GS1 was attenuated in the presence of glutamine, which suggested that GS1 is feedback inhibited (FIG. 16).

These results demonstrate the successful engineering of a glutamine auxotroph Lemna line.

### Example 3

### Genetic Engineering of a Lemna Biotin Auxotroph

Two approaches were used to generate Lemna biotin auxotroph lines. In the first approach, constructs overexpressing the biotin-binding protein streptavidin were utilized to essentially titrate out endogenous biotin and generate an zuxotrophic requirement for this vitamin. In the second approach, an RNAi construct similar to that described in Example 1 was utilized to knockdown expression of biotin synthase.

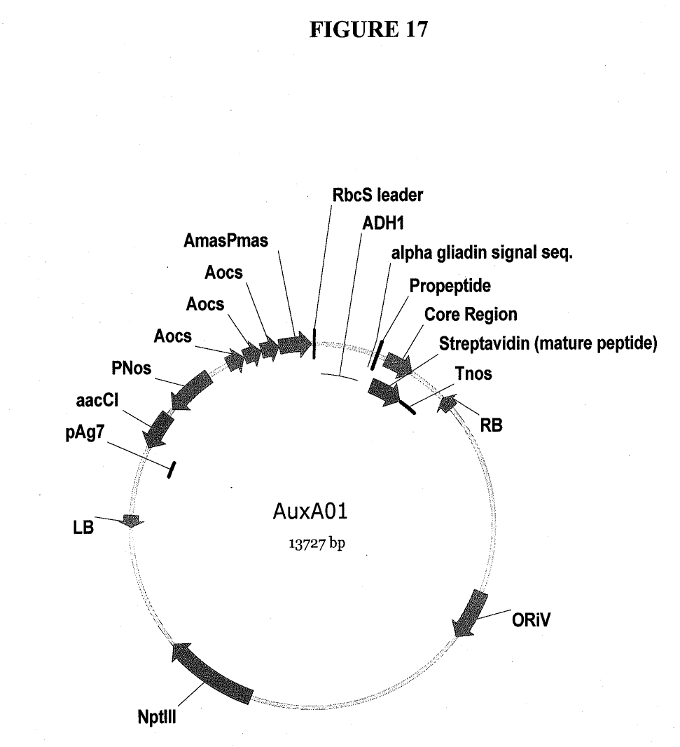

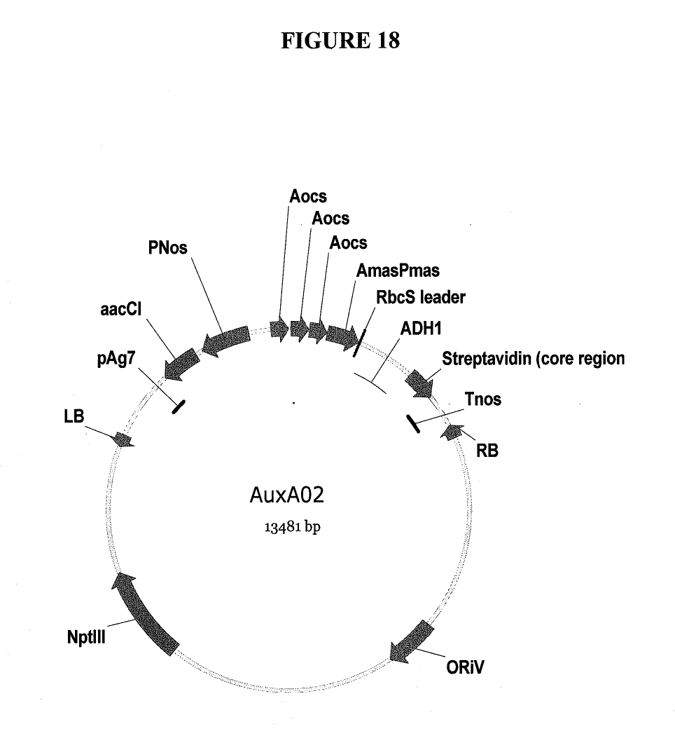

Stretavidin expression vectors AUXA01 (FIG. 17) and AUXA02 (FIG. 18) were designed. AUXA01 contains the Superpromoter driving expression of the mature streptavidin protein with an α-gliadin signal sequence, and AUXA02 contains the Superpromoter driving expression of a core region of streptavidin.

For RNAi suppression of biotin synthase, cDNAs encoding two isoforms of a biotin synthase were cloned from Lemna minor using degenerate primer PCR with primers designed from amino acid sequence alignments of published biotin sequences from other plant species. The full-length cDNA for L. minor BS isoform #1 and L. minor BS isoform #2 are set forth in SEQ ID NOs: 19 and 22, respectively. An RNAi construct was designed based on the cDNA for BS isoform #1 (SEQ ID NO:19), using a strategy similar to that for TD (see TD schematic shown in FIG. 6). This RNAi construct was cloned into two vectors to generate the AUXB01 (FIG. 19) and AUXB02 (FIG. 20) RNAi vectors. The AUXB01 RNAi vector uses the Superpromoter to drive expression of the BS RNAi construct. The AUXB02 RNAi vector uses the Spirodela polyrrhiza (SpUbq; SEQ ID NO:40) promoter to drive expression of this BS RNAi construct.

Transgenic Lemna plants were generated and maintained in a manner similar to that described above. Following Agrobacterium-mediated transformation, Agrobacterium co-cultivation was performed. Lemna nodules were then maintained on selection medium with varying levels of geneticin and auxotrophic supplement as shown in Table 7 below.

TABLE 7
Biotin Synthase Transformation and Selection Conditions.

| | | Selection (mg/L | |
|---|---|---|---|
| Gene Target | Construct | geneticin) | Biotin (mM) |
| BS | AUXA01 | 7.0 | 0.25, 1.0 |
| BS | AUXA02 | 7.0 | 0.25, 1.0 |
| BS | AUXB01 | 7.0 | 0.25, 1.0 |
| BS | AUXB02 | 5.5, 6.5 | 0.25, 1.0 |

Transgenic plant lines were successfully generated from these transformations. After harvest, plant lines were maintained in standard Lemna growth medium with appropriate supplementation with 0.25 mM biotin. The number of lines generated per transformation are shown in Table 8.

TABLE 8
Biotin Synthase Auxotrophic Lines Generated Per Transformation

| Auxotrophic | | |
|---|---|---|
| Requirement | Construct | Total Lines Generated |
| Biotin | AUXA01 | 127 |
| Biotin | AUXA02 | 120 |
| Biotin | AUXB01 | 71 |
| Biotin | AUXB02 | 32 |

A primary screening process similar to that described in Example 1 was carried out to evaluate the phenotype of these auxotroph plant lines. Plant lines were initially screened in 12-well plates where auxotroph transformants were grown with and without biotin supplementation. Lines that exhibited poor growth in standard media and subsequent recovery in the presence of supplement were selected for secondary screening. For secondary screening the standard format was used with plant lines being grown in IV's for 14 days.

### Results

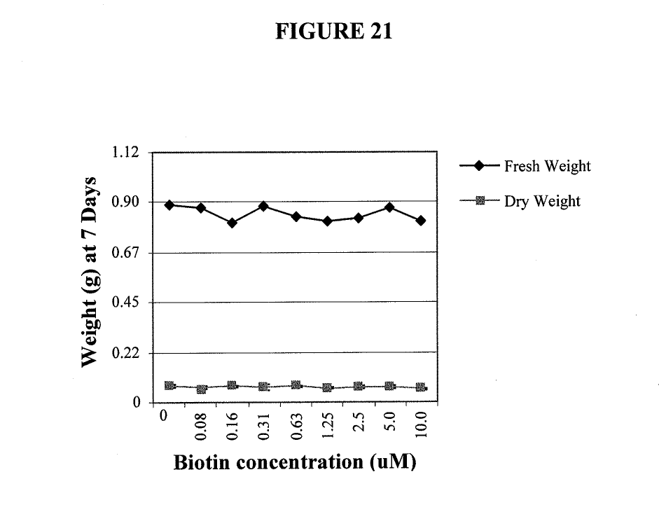

Initial experiments were conducted to determine the tolerance range of wild-type Lemna minor plants to biotin. As shown in FIG. 21, biotin alone had no effect on accumulation of plant biomass in wild-type plants after seven days.

Primary screening of Lemna minor AUXA01 and AUXA02 transformants grown with 0.25 mM biotin or without biotin, when compared to similarly grown wild-type Lemna minor plants, showed several lines with the desired auxotrophic phenotype. Secondary screening of Lemna minor AUXA01 transformants (including screening of lines 32 and 42) in the absence of biotin (0 mM biotin) or in the presence of 0.25 mM or 0.75 mM biotin in the growth medium, when compared to similarly grown wild-type Lemna minor plants, showed that the transformed plants exhibited almost a full recovery to a wild-type phenotype. Similar results were observed in a secondary screening of Lemna minor AUXA02 transformants (including screening of lines 24, 42, 72, and 108) grown in 0 mM, 0.25 mM, or 0.75 mM biotin, when compared to wild-type plants. In the absence of biotin, these transformed plants exhibited poor growth and plant health.

Likewise, primary screening of Lemna minor AUXB01 and AUXB02 transformants grown with (+) or without (−) 0.25 mM biotin, when compared to similarly grown wild-type Lemna minor plants, showed several lines with the desired auxotroph phenotype. Secondary screening of Lemna minor AUXB01 transformants (including screening of line 1 and line 16) in the absence of biotin (0 mM biotin) or in the presence of 0.25 mM or 0.75 mM biotin in the growth medium, when compared to similarly grown wild-type plants, showed that the transformed plants exhibited almost a full recovery to a wild-type phenotype. Similar results were observed in a secondary screening of Lemna minor AUXB02 transformants (including screening of line 8) grown in 0 mM, 0.25 mM, or 0.75 mM biotin in the growth medium when compared to similarly grown wild-type Lemna minor plants. In the absence of biotin, these transformed plants also exhibited poor growth and plant health. These results demonstrate the successful engineering of Lemna biotin auxotroph lines.

### Example 4

### Listing of Sequence Identifiers

Table 9 below provides a summary of the TD, GS, and BS sequences referred to herein and provided the Sequence Listing for this application.

TABLE 9
Sequence Identifiers for Lemna minor TD, GS, and BS sequences.

| Sequence Identifier | Description |
|---|---|
| SEQ ID NO: 1 | Full-length cDNA for L. minor threonine |
| | deaminase isoform #1 |
| SEQ ID NO: 2 | CDS for L. minor threonine deaminase |
| | isoforms #1 |
| SEQ ID NO: 3 | Predicted amino acid sequence for |
| | threonine deaminase isoform #1 |
| SEQ ID NO: 4 | Full-length cDNA for L. minor threonine |
| | deaminase isoform #2 |
| SEQ ID NO: 5 | CDS for L. minor threonine deaminase |
| | isoform #2 |
| SEQ ID NO: 6 | Predicted amino acid sequence for L. minor |
| | threonine deaminase isoform #2 |
| SEQ ID NO: 7 | Full-length cDNA for L. minor glutamine |
| | synthetase 1 (GS1) isoform. #1 |
| SEQ ID NO: 8 | CDS for L. minor glutamine synthetase 1 |
| | (GS1) isoform #1 |
| SEQ ID NO: 9 | Predicted amino acid sequence for L. minor |
| | glutamine synthetase 1 (GS1) |
| | isoform #1 |
| SEQ ID NO: 10 | Full-length cDNA for L. minor glutamine |
| | synthetase 1 (GS1) isoform #2 |
| SEQ ID NO: 11 | CDS for L. minor glutamine synthetase 1 |
| | (GS1) isoform #2 |
| SEQ ID NO: 12 | Predicted amino acid sequence for |
| | glutamine synthetase 1 (GS1) isoform #2 |
| SEQ ID NO: 13 | Full-length cDNA for L. minor glutamine |
| | synthetase 2 (GS2) isoform #1 |
| SEQ ID NO: 14 | CDS for L. minor glutamine synthetase 2 |
| | (GS2) isoform #1 |
| SEQ ID NO: 15 | Predicted amino acid sequence for L. minor |
| | glutamine synthetase 2 (GS2) |
| | isoform #1 |
| SEQ ID NO: 16 | Full-length cDNA for L. minor glutamine |
| | synthetase 2 (GS2) isoform #2 |
| SEQ ID NO: 17 | CDS for L. minor glutamine synthetase 2 |
| | (GS2) isoform #2 |
| SEQ ID NO: 18 | Predicted amino acid sequence for L. minor |
| | glutamine synthetase 2 (GS2) |
| | isoform #2 |
| SEQ ID NO: 19 | Full-length cDNA for L. minor biotin |
| | synthase isoform #1 |
| SEQ ID NO: 20 | CDS for L. minor biotin synthase isoform |
| | #1 |
| SEQ ID NO: 21 | Predicted amino acid sequence for L. minor |
| | biotin synthase isoform #1 |
| SEQ ID NO: 22 | Full-length cDNA for L. minor biotin |
| | synthase isoform #2 |
| SEQ ID NO: 23 | CDS for L. minor biotin synthase isoform |
| | #2 |
| SEQ ID NO: 24 | Predicted amino acid sequence for biotin |
| | synthase isoform #2 |

Tables 10 and 11 summarize the relationships between the TD, GS, and BS isoforms at the nucleotide and amino acid levels.

TABLE 10
Nucleotide and Amino Acid Sequence Identities for L. minor TD, GS,
and BS isoforms.

| | | % | |
|---|---|---|---|
| | | Nucleotide | |
| Target | Isoform# | Identity | % Amino acid Identity |
| Threonine deaminase | 1 and 2 | 99.7 | 71.4 (whole sequence) |
| | | | 99.6 (region of overlap) |
| Glutamine synthetase | 1 and 2 | 96.5 | 97.8 |
| 1 (GS1) | | | |
| Glutamine synthetase | 1 and 2 | 98.4 | 99.1 |
| 2 (GS2) | | | |
| Biotin synthase | 1 and 2 | 99.7 | 99.5 |

TABLE 11
Nucleotide and Amino Acid Sequence Identities for L. minor
GS1 and GS2 isoforms.

| | | | |
|---|---|---|---|
| GS1 | | GS2 | |
| isoform 1 | GS1 isoform 2 | isoform 1 | GS2 isoform 2 |

% Nucleotide Identity for 4 Isoforms of Glutamine Synthetase 1 and 2

| | | | |
|---|---|---|---|
| GS1 isoform 1 | 97 | 70 | 70 |
| GS1 isoform 2 | | 70 | 70 |
| GS2 isoform 1 | | | 98 |
| GS2 isoform 2 | | | |

% Amino Acid Identity for all 4 isoforms of Glutamine synthetase 1 and 2

| | | | |
|---|---|---|---|
| GS1 isoform 1 | 98 | 79 | 79 |
| GS1 isoform 2 | | 79 | 79 |
| GS2 isoform 1 | | | 99 |
| GS2 isoform 2 | | | |

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

It is to be understood that the term “about” as used herein means within a statistically meaningful range of a value such as a stated concentration range, time frame, molecular weight, temperature, or pH. Such a range can be within an order of magnitude, typically within 20%, more typically still within 10%, and even more typically within 5% of a given value or range. The allowable variation encompassed by “about” will depend upon the particular system under study, and can be readily appreciated by one of skill in the art.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

1-11. (canceled)

12. A method for biocontaining a transgenic duckweed plant, plant cell, or nodule, wherein said transgenic duckweed plant, plant cell, or nodule comprises a heterologous polynucleotide of interest, said method comprising the steps of:
providing an effective amount of an essential compound to said transgenic duckweed plant, plant cell, or nodule, wherein said transgenic duckweed plant, plant cell, or nodule has an auxotrophic requirement for said essential compound, and
removing said essential compound from said transgenic duckweed plant, plant cell, or nodule, wherein growth of said transgenic duckweed plant, plant cell, or nodule is inhibited in the absence of said compound, whereby said transgenic duckweed plant, plant cell, or nodule is biocontained;
wherein the compound is an essential amino acid, a carbohydrate, a fatty acid, a nucleic acid, a vitamin, a plant hormone, or a precursor thereof.

13. (canceled)

14. (canceled)

15. The method of claim 12, wherein said transgenic duckweed plant, plant cell, or nodule is stably transformed with a polynucleotide construct having a nucleotide sequence that is capable of inhibiting expression or function of a component of a biosynthetic pathway for said essential compound, said nucleotide sequence being operably linked to a promoter that is functional in a plant cell.

16. The method of claim 15, wherein said nucleotide sequence encodes a polypeptide that inhibits function of said component of said biosynthetic pathway.

17. The method of claim 16, wherein said polypeptide is an antibody or a binding protein that binds said component of the biosynthetic pathway for said essential compound, thereby inhibiting function of said component.

18. The method of claim 17, wherein said component of said biosynthetic pathway is biotin synthase.

19. The method of claim 18, wherein said nucleotide sequence encodes streptavidin or a fragment thereof that binds biotin synthase, thereby inhibiting function of said biotin synthase.

20. (canceled)

21. (canceled)

22. The method of claim 15, wherein said nucleotide sequence encodes an inhibitory nucleotide molecule that is capable of being transcribed as an inhibitory polynucleotide selected from the group consisting of a single-stranded RNA polynucleotide, a double-stranded RNA polynucleotide, and a combination thereof.

23. The method of claim 22, wherein said essential compound is an amino acid.

24. The method of claim 23, wherein said amino acid is isoleucine.

25. (canceled)

26. (canceled)

27. The method of claim 24, wherein said component of said biosynthetic pathway is threonine deaminase (TD), and wherein said nucleotide sequence comprises a sequence selected from the group consisting of:
(a) the nucleotide sequence set forth in SEQ ID NO: 1 or 2, or a complement thereof;
(b) the nucleotide sequence set forth in SEQ ID NO:4 or 5, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

28. The method of claim 23, wherein said amino acid is glutamine.

29-31. (canceled)

32. The method of claim 28, wherein said component is GS1, and wherein said nucleotide sequence comprises a sequence selected from the group consisting of:
(a) the nucleotide sequence set forth in SEQ ID NO:7 or 8, or a complement thereof;
(b) the nucleotide sequence set forth in SEQ ID NO: 10 or 11, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

33. The method of claim 28, wherein said component is GS2, and wherein said nucleotide sequence comprises a sequence selected from the group consisting of:
(a) the nucleotide sequence set forth in SEQ ID NO: 13 or 14, or a complement thereof;
(b) the nucleotide sequence set forth in SEQ ID NO: 16 or 17, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

34. The method of claim 28, wherein said component is a combination of said GS1 and said GS2, and wherein said nucleotide sequence comprises a fusion polynucleotide that is capable inhibiting expression of said GS1 and said GS2 in said duckweed plant or duckweed plant cell or nodule, wherein said fusion polynucleotide comprises in the 5′-to-3′ orientation and operably linked:
(a) a chimeric forward fragment, said chimeric forward fragment comprising in either order:
(i) a first fragment comprising about 500 to about 650 contiguous nucleotides having at least 90% sequence identity to a nucleotide sequence of about 500 to about 650 contiguous nucleotides of a polynucleotide encoding said GS1; and
(ii) a second fragment comprising about 500 to about 650 contiguous nucleotides having at least 90% sequence identity to a nucleotide sequence of about 500 to about 650 contiguous nucleotides of a polynucleotide encoding said GS2;
(b) a spacer sequence comprising about 200 to about 700 nucleotides; and
(c) a reverse fragment, said reverse fragment having sufficient length and sufficient complementarity to said chimeric forward fragment such that said fusion polynucleotide is transcribed as an RNA molecule capable of forming a hairpin RNA structure.

35. The method of claim 22, wherein said essential compound is a vitamin, and wherein said vitamin is biotin.

36. (canceled)

37. (canceled)

38. The method of claim 35, wherein said component of said biosynthetic pathway is biotin synthase (BS), and wherein said nucleotide sequence comprises a sequence selected from the group consisting of:
(a) the nucleotide sequence set forth in SEQ ID NO: 19 or 20, or a complement thereof;
(b) the nucleotide sequence set forth in SEQ ID NO:22 or 23, or a complement thereof;
(c) a nucleotide sequence having at least 90% sequence identity to the sequence of preceding item (a) or (b); and
(d) a fragment of the nucleotide sequence of any one of preceding items (a) through (c), wherein said fragment comprises at least 75 contiguous nucleotides of said nucleotide sequence.

39-97. (canceled)

98. A method for biocontaining a transgenic duckweed plant, plant cell, or nodule, wherein said transgenic duckweed plant, plant cell, or nodule comprises a heterologous polynucleotide of interest, said method comprising the steps of:
providing an effective amount of an essential compound to said transgenic duckweed plant, plant cell, or nodule, wherein said transgenic duckweed plant, plant cell, or nodule has an auxotrophic requirement for said essential compound, and
removing said essential compound from said transgenic duckweed plant, plant cell, or nodule, wherein growth of said transgenic duckweed plant, plant cell, or nodule is inhibited in the absence of said compound, whereby said transgenic duckweed plant, plant cell, or nodule is biocontained;
wherein said essential compound is isoleucine, glutamine, or biotin.

99-102. (canceled)

103. A method of regulating production of a heterologous polypeptide of interest in a transgenic duckweed plant, plant cell, or nodule having an auxotrophic requirement for isoleucine, glutamine, or biotin, wherein said transgenic duckweed plant, plant cell, or nodule comprises a heterologous polynucleotide encoding said polypeptide of interest operably linked to a promoter that is functional in a plant cell, said method comprising:
providing an effective amount of said isoleucine, glutamine, or biotin to said transgenic duckweed plant, plant cell, or nodule under culture conditions suitable for expression and production of said heterologous polypeptide, wherein said transgenic duckweed plant, plant cell, or nodule grows in the presence of said effective amount of said isoleucine, glutamine, or biotin and said heterologous polypeptide is produced; and
removing said isoleucine, glutamine, or biotin from said transgenic duckweed plant, plant cell, or nodule, wherein growth of said transgenic duckweed plant, plant cell, or nodule is inhibited in the absence of said isoleucine, glutamine, or biotin, whereby expression and production of said heterologous polypeptide is reduced.

104. A duckweed plant, plant cell, or nodule having an auxotrophic requirement for isoleucine, glutamine, or biotin.

105. The duckweed plant, plant cell, or nodule of claim 104, wherein said duckweed plant, plant cell, or nodule comprises a heterologous polynucleotide, wherein said polynucleotide comprises a coding sequence for a heterologous polypeptide of interest operably linked to a promoter that is functional in a plant cell.
